SEQUENCE LISTING (36 sequences)

SEQ ID NO: 1 - DNA, 2059 bp, Homo sapiens
atcgcgagatcaggaaggtggccgagtgtgtcgccgcggccatcaggcacttctccttcctgcccttgtatgaagaaggatgtgtttgcttccccttgtgccatgattgtaaatttcctgaggcctcctcagccctgcggaactggctagagcaatgtatcttaggctcacttaaggaagctgtagagatgagcccaaggagggaaaccagaagagccccccaggctcaccagttgtttgttggctccctacaaacatgtcattcaagtggctaatcttacaacagcacaaattcatctaaccagaaagagaagaggaggctccaaaggcacttgactactgagcatcaccctggacgtgtacaagtctgcgtccttattgttttcttcattgggccgaactttctggtcctcatccaacagctcttctatcatgtgttcgaaagtgtcagccaatgatgtcaagcctcttgaacctgccttgggcccattcacgctctccagagtcccatgggtccgcacacctggagatactctattatagcaaagaagaaagataatttcattgagccatcctgttttacagcacccaacagaatcccttcaaagcctcgtggtctgacaccctatgctacgtgacttgtgacccatccatttgtcatgttcttcgggaatgtggctaaggggctaagatgtgacttgaaaagaaaggtagaacaagatcatctcaaatttattatcaaggaatagttcagaaaacgacttcagaccacagagacagcagaacagatggtccggcatggatagagcatcagacactcacagactgtgccaacaagagccatcgagtcaaaacagccaaaggaaggagggtcatggaatgggttctctcacaccaaactgatgcccagaggccctcagcatgaataacaaaggcaaccagacccacaagccatactgagtggatacaaaacctatacctaagctgacatcccaaatgtgtgtggcaagttagatgatgatggcacaaaagacagaacaccttgctttctggccattgtcagctcttggaagagagcacacttttagaggagcagctgcaaggaccctgagaacaaaactggaaatgtctgttatgaaagccttcacaggaaattctgcaagtggcaacgtgggtccattccgtgtgtgtcactagagctggcgcaagcccatggccatggtgaggcagcgtttccactggaactaatctgatacctgcaccagctcttgcaactgtgcagtgttcccactgcaaactacggatgggagaggataaagaacttcaatctttaaaaaagagaggattttccctcctggtgagtcaaaatgaacaagaaataccccaggacctcccttccctccttggccattaatgagatgaaggcaattaactcacatagtataaatgaatcatttgaggtgatgactgcattttaggcaaatgatgactttcttggttccattggtttgcaagtaaaagttacacacattgaaaagacactgaaacagatttcctaaatgcttcattttctggatgcaccaatgttgacctactatacatgttaaatggttttaaaatatcaccttaaaataaaggaaacttccagctactaactcagctctgaatgggctatgaaaggctccaaaggtatgtgaaaaattactgttattttgctttaaaaaatgtgatgtctaagagtgtctgcaatgttctaatgcttcaaaacatgtacgtaagccttgtttatctggaaatcatttctttctgcttatatcatttataaatagaaaatgttctgtaataacttaaaatagttccacatacataatgcttttcgtgtcataatacttactactggtctatatttaccaacatttatcacattttacaaaatgaagtagaagaaaaaaaagacaacgactttatggccctggaattccagtaatggtgaccaacatgttttaaattccagtaaaggttatggttacatttcaaaaaaaaaaaaaaaaaaaaaaaa

SEQ ID NO: 2 - DNA, 4755 bp, Homo sapiens
cacacacacacacacacacagagagaacatctctagtaaaaagaaaagttgagctttcttagctagatgtgtgtattagccagaaaaagccaaggagtgaagggttttagagaactggaggagataaagtggagtctgcatatgggaggcatttgaaatggacttaaatgtctttttaatgctgactttttcagttttctccttaccagacacattgttttcatgacattagccccaggcatagacacatcattaaaatgaacatgtcaaaaaatgatttctgtttagaaataagcaaaacattttcagttgtgaccacccaggtgtagaataaagaacagtggaattgggagccctgagttctaacataaactttcttcatgacataaggcaagtcttctatggcctttggtttccttacctgtaaaacaggatggctcaatgaaattatctttcttctttgctataatagagtatctctgtgggaagaggaaaaaaaaagtcaatttaaaggctccttatagttccccaactgctgttttattgtgctattcatgcctagacatcacatagctagaaaggcccatcagacccctcaggccactgctgttcctgtcacacattcctgcaaaggaccatgttgctaacttgaaaaaaattactattaattacacttgcagttgttgcttagtaacatttatgattttgtgtttctcgtgacagcatgagcagagatcattaaaaattaaacttacaaagctgctaaagtgggaagaaggagaacttgaagccacaatttttgcacttgcttagaagccatctaatctcaggttatatgctagatcttgggggcaaacactgcatgtctctggtttatattaaaccacatacagcacactactgacactgatttgtgtctggtgcagctggagtttatcaccaagacataaaaaaaccttgaccctgcagaatggcctggaattacaatcagatgggccacatggcatcccggtgaaagaaagccctaaccagttttctgtcttgtttctgctttctccctacagttccaccaggtgagaagagtgatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatagtggatttatgttgtatagattagattatattgagacaaaaattatctatttgtatatatacataacagggtaaattattcagttaagaaaaaaataattttatgaactgcatgtataaatgaagtttatacagtacagtggttctacaatctatttattggacatgtccatgaccagaagggaaacagtcatttgcgcacaacttaaaaagtctgcattacattccttgataatgttgtggtttgttgccgttgccaagaactgaaaacataaaaagttaaaaaaaataataaattgcatgctgctttaattgtgaattgataataaactgtcctctttcagaaaacagaaaaaaacacacacacacacaacaaaaatttgaaccaaaacattccgtttacattttagacagtaagtatcttcgttcttgttagtactatatctgttttactgcttttaacttctgatagcgttggaattaaaacaatgtcaaggtgctgttgtcattgctttactggcttaggggatgggggatggggggtatatttttgtttgttttgtgtttttttttcgtttgtttgttttgttttttagttcccacagggagtagagatggggaaagaattcctacaatatatattctggctgataaaagatacatttgtatgttgtgaagatgtttgcaatatcgatcagatgactagaaagtgaataaaaattaaggcaactgaacaaaaaaatgctcacactccacatcccgtgatgcacctcccaggccccgctcattctttgggcgttggtcagagtaagctgcttttgacggaaggacctatgtttgctcagaacacattctttccccccctccccctctggtctcctctttgttttgttttaaggaagaaaaatcagttgcgcgttctgaaatattttaccactgctgtgaacaagtgaacacattgtgtcacatcatgacactcgtataagcatggagaacagtgatttttttttagaacagaaaacaacaaaaaataaccccaaaatgaagattattttttatgaggagtgaacatttgggtaaatcatggctaagcttaaaaaaaactcatggtgaggcttaacaatgtcttgtaagcaaaaggtagagccctgtatcaacccagaaacacctagatcagaacaggaatccacattgccagtgacatgagactgaacagccaaatggaggctatgtggagttggcattgcatttaccggcagtgcgggaggaatttctgagtggccatcccaaggtctaggtggaggtggggcatggtatttgagacattccaaaacgaaggcctctgaaggacccttcagaggtggctctggaatgacatgtgtcaagctgcttggacctcgtgctttaagtgcctacattatctaactgtgctcaagaggttctcgactggaggaccacactcaagccgacttatgcccaccatcccacctctggataattttgcataaaattggattagcctggagcaggttgggagccaaatgtggcatttgtgatcatgagattgatgcaatgagatagaagatgtttgctacctgaacacttattgctttgaaactagacttgaggaaaccagggtttatcttttgagaacttttggtaagggaaaagggaacaggaaaagaaaccccaaactcaggccgaatgatcaaggggacccataggaaatcttgtccagagacaagacttcgggaaggtgtctggacattcagaacaccaagacttgaaggtgccttgctcaatggaagaggccaggacagagctgacaaaattttgctccccagtgaaggccacagcaaccttctgcccatcctgtctgttcatggagagggtccctgcctcacctctgccattttgggttaggagaagtcaagttgggagcctgaaatagtggttcttggaaaaatggatccccagtgaaaactagagctctaagcccattcagcccatttcacacctgaaaatgttagtgatcaccacttggaccagcatccttaagtatcagaaagccccaagcaattgctgcatcttagtagggtgagggataagcaaaagaggatgttcaccataacccaggaatgaagataccatcagcaaagaatttcaatttgttcagtctttcatttagagctagtctttcacagtaccatctgaatacctctttgaaagaaggaagactttacgtagtgtagatttgttttgtgttgtttgaaaatattatctttgtaattatttttaatatgtaaggaatgcttggaatatctgctatatgtcaactttatgcagcttccttttgagggacaaatttaaaacaaacaaccccccatcacaaacttaaaggattgcaagggccagatctgttaagtggtttcataggagacacatccagcaattgtgtggtcagtggctcttttacccaataagatacatcacagtcacatgcttgatggtttatgttgacctaagatttattttgttaaaatctctctctgttgtgttcgttcttgttctgttttgttttgttttttaaagtcttgctgtggtctctttgtggcagaagtgtttcatgcatggcagcaggcctgttgcttttttatggcgattcccattgaaaatgtaagtaaatgtctgtggccttgttctctctatggtaaagatattattcaccatgtaaaacaaaaaacaatatttattgtattttagtatatttatataattatgttattgaaaaaaattggcattaaaacttaaccgcatcagaacctattgtaaatacaagttctatttaagtgtactaattaacatataatatatgttttaaatatagaatttttaatgtttttaaatatattttcaaagtacataaaa

SEQ ID NO: 3 - DNA, 361 bp, Homo sapiens
tttttttttttcagattttcctatgtatttttattttttccagatcgttaaaaaaaaaaattaatgtgttagcattctggttggaaatgtattatatttacagattaatctaggggaacatctatttaaaatgtcaaatcatctcgttcaagaacaaggtatagtatagtccctcggtacctgagggagattggttctaggacccccatagatactcaaatcccaggatgctcaagtacctgatataaaatggtgtagtatttgcatataacctatgcacatcttcccgtataaatcatctctatattatttataatacttaaaagtgtgaatactatataattatattaacttatatttt

SEQ ID NO: 4 - DNA, 6469 bp, Homo sapiens
ggcgcttcctgttccggcgccaggaggagccgcgcgctgctggtgctgttgccgccgctgctctagctgccgtcagtcaggctgcgcccgcgtcttcagggcccagtccctcggacccatcgccgcttctagaccctactgcggtctcggatattgccgggaaaatgtctgatgaattttcgttggcagatgcactacctgaacactcccctgccaaaacctctgctgtgagcaatacaaaacctggccaacctcctcaaggctggccaggctccaacccttggaataatccgagtgctccatcttcagtgccatctggactcccaccaagtgcaacaccctccactgtgccttttggaccagcaccaacaggaatgtatccctccgtgcctcccaccggaccacctccaggacccccagcaccctttcctccttccggaccatcatgtcccccacctggtggtccttatccagccccaactgtgccgggccctggccccacagggccatatcctacaccaaatatgccctttccagagctacccagaccatatggtgcacccacagatccagctgcagctggtcctttaggtccatggggatccatgtcttctggaccttgggcgccaggaatgggagggcagtatcctacccctaatatgccatatccatctccaggcccatatcccgctcctcctcctccccaagcccctggggcagcaccacctgttccatggggcaccgttccaccaggagcctggggaccaccagcaccatatcctgcccctacaggatcgtatcccacaccaggactctatcctactcccagtaatcctttccaagtgccttcaggaccttctggtgctccaccaatgcctggtggcccccattcttaccattaagttaacaatggacgaagagatgacgctttgctttttgaagtacatgtatatgcacatgaatgcatatataaaaattgctggtttcactattagagggcattcatgaaagaacaactcttgcacctctcagagaagataactgcctcttgtacttggatgcgtagtacatcatatgtatacaatcagataaaagcatagaagtaaatcattcggatgtgatttttatttggttttcatggaaagttaaagtgataaagtatattgaatagttctttgacagaatttgtttaaactatgaaactacacacttaaaaatctaagatgtggattattgttagaatctgcaacttcattggcaaattatttcaagtatttttctataatcactttccccttctaaataaataaacttcgagaataacccatcataatccaaacaaatgatgcctcaacattttgagctgctctgtcggacaaataaacctggtcctcttgaggttatattttggatatacatttttaaactgtcagtaattattgtcagatgtggagttcaatagccagccagtgttcatttttatccttgagcttttagtaaaaacttcctggttttatttttagtcattgggtcatacagcactaaagtctgctatttatggaaactaacttttttgtttttaatccaggccaacatgtatgtaaattaaatttttagataattgattatctctttgtactacttgagatttgattatgagatgtgcatattgctttgggaagagctcgaggaaggaaataattctctcctttgttttgaacctcaaactagataaaccctaggaattgcttaactgcaacaagtaattttcattcccacaaaaacctgaggcagctcttttgcccagagcgttccctgtagccacccccaccccacttgcccttggttctttagaaggagcacacacatcccttgattcctccctgatgtggtaaactggcacactccaggggtctaaaacataaaacagttgtgtttagggaaccttaagtcatgcagacatgactgttctctttgtacaagtgtgaatcaaaatatgtatctctttttcagagtctggttaagctatgtcattgtctactgcatagtttcctgagtctgtttgtaaagtgcttatggctaacagttcagttctgtatttgttgacaggtaaataagtggagttgagtgccatctttgaaaaaattaccctctagctctaacactgaaaataataataaattgtagatctctgcaactaagtttaaagcagtgtgactgtgttgcttaaatatcaagtattgtttataaccaccaaaaaaaaaaagccctggtagttttttggcaccttatgtttaaatcagattcttagatttggagtagacctgaccttgttatttattagataacattttgaatgtatccattggatttctaaaatgtattgtgaatttctcagacaaacaggatttatgctggagctctgttttgcttagaaataaaatatttagtagtttatttctgctctaattaaaatgtcaagaatgccaaatgctgccagttttttggtttgatagctacctccttctaagaaagcaaaatggttacctttgagaggaacattcagtgtttaatcatcccttatgttaactagatgatagattcaagcttttagaaatgagaaagtagaaactaatttgttaagatattttcagactgcggaatgttgttagctttttctttcacttctcttcaaggacaggtgttagctgtctacaatactgttgaactctgttgtcaaagtagcccccttagtctacaaggcaggtagccttggcttgaattatcaatatcaaaatgtcagttaaccatggagggataaagtaatgtgaaaagtgagatggctgcaaagatagctctccttacagttattttggctgtcctacattgggataagctgacaaattagcagtatttagtttaacactggagcaaatataatttgagtaggaagaagagatagcaggtttgggaatctataattatgaagtccattgattttgggagaaaatctgttgctaaaggatttgaagggccatgaacacaatttgggattattactccctataagtataataattttgctagtgacccatactgtccagtgtgccctaaatcatactgctattgtactccctttgttttcaaggactttgcaactggtatttgggggagatttttttttttttttgagacggagtctcgctctgtcgcccatgctggagtgcagtggtgctatcttggttcactgcaagctccatctcccaggttcacaccattctcctgcctcagcctcccaagcagctgggactacaggtgcccgccaccatgcccggctaatttttttttttttttttttttagtagagatggggtttcactgtgttagccaggatggtctcgatctcctgacctcgtgatctgcccgccttggcttcccaaagtgctgggattacaggcgtgagccaccacgtccggccgatttttttttttttttttaatgtaagaatggagataaaagggataatataatttgcttttatattgttatttttgtaaagcatcttttcttcaattcttgttggcattctgggccaaaatatttcaggttggttcggtgtggagttaagaaaagcaggcgttttagtggagaaatggggaacagcatcaagaaaggcttttttccttttttcttttttttttggagacagagtcttgccctgtcacccaggctggagtgcaatggcgtgatcttggctcgctgcaacctctgcctccaggttcaaacgattcttctgcctcagcctcccaagtagctgggattacaggtgcccgccaccacacccatttttgtatttttagtagagacgggggtttcaccatgttggccagggtggtctgaaactcctgacctcgtgatccgcctgcctcagcctcccaaagtgctgagattacaggcgtgagccaccatgcgtgacctttttttctttttaaaagggaacaatgttgctttcaaaacaagacatgctaggctgaaactgatttatggaaaagactgcttgttagcaagtatatttggtcttgagggggatacagattatagaatatgctgacatttgggcttcagaggaagaattttcaaatctaatggaaatagttgaggtgttcaggaatgctgtttcttggagttggaagcttaggttttgaaatgttgaaaccaaaaagacaaaaattaaaacatagaccttaggtcgtcattcacacccggttctcaagaatcaagtggagcacttcaaagaccttggcttgtctgtcccatcctgccactttctcatcttttcatgcttttgaagacaccatttacagctctgactcagccctattttgtgtaaagtaatatattgattattcagaaatagacaatacattttttaattacccaaggactgactgttttgtgcattttactgttggttgtcttcagtagagaatagtaatagggcagagaaaagtatatattttgcctcagtcagtcccaccaccacaatggactattgggatattttctaaaaaaccaatcaatttgcccatgattacctcacaaataattagtgctacctggggtactctcaaatatacagcttttgaaactgtagatgaaaaaagctctactcagagtttttgtcaagactgtgcctgggttgaatatcagtcaattgcctacacttctaaacaataagtgccaatgtctcaattttctcaccctgaatgatagaagctagctttatcaaatgccaaggttagaaagcctggaaataaaacttaagcacagacattcaagtttttgaaaagcataagcctaaattcagataaatcacactgatatattgtactatgcatagaaagttgtaggtggcgttcagggaagactttgattttaataaagcaatatttagtattgaagacaaacactttttattttcagatttctgccaagtaaaacagaaattgccaataaaataatcagtattttgtaaatggcaggcaagcttctggctgtcgaaaacatctgagtcatttattcagtagacaatatgtccttgatccaggttctttgccagctataagggaatccctgtccttgagaggctcatagtctataagtaacattacagaatttgttagcatacccattcattattagttttacctaaacgtgttaggatcactactggtggaaattgtaaccagcctttgggcatcttaaagggtgacatgtggcatgcctttttttttttttaagaatttaatgtttttcaagattgtagtgttgatcagcgcaacaattcaagtgtgcaaagtaacaggatagtttgcctcttcactttacccctggataaaggcactttcactgcctgtcactgatcagcagatactgacttgttgccattaagtgaacttgacttcttatgtgtgctctatgagtttgttgtaattttcttcttgaaattgtgatttttcactgacagtaatgacaaatttaatgtatgtaattgtctatgcattttaagttaaactgcctaaaatgtgatttgagacatatacatatgtttgtattataaattgtaagcaatcagtttgagatactaggttttatcacctgctgctgtatttgtaaacaaagacaaatgttgctttaagaagtaattataattaggaataggctatggatgtgatacttggtattttttaagataaacttgtttgcttttgtgtattatacctggaaactttttttaaaaaatgtattttcatggtttcacagatttttcatgttattttattctttaggcccaattctgggcttctctgagcaagtccagagcctaattaactgtaaatttgttgtcaaaaaggaagaaaaaagggcctgagatacctctttgcatgtgacctgcattcactaaggatatctggaaaccacccttcctccgcaaaccctctcagcaacatggtgtccattgtggtgattttctcttcttttaaggctaggctactcttggtaaccagattatccgtatatatgataatatgaagtcagggaactttctctgtctgtccctactcccctcactcccccactttctgttatgaaagatagttctacttttatcattaactgctacgcatttagtgagggtcacattattaaacttggagtttaccattttcccacaggagatttcgctggcattccttggaactcccaatttcagtagggcaatgaatgaatgaatactttgcagtgctacttttggaaggaatttctgctttttgccttatgattggacaaaatgcagctgtaaaattttaaattgtttttgatatgttattcaatatcccatgaaagtattcacctaaagtggagttatgaaatggatggtgaaataataagaccattctggagcagg

SEQ ID NO: 5 - DNA, 2042 bp, Homo sapiens
agagctgcttgcttaaaagactctggcctccctcttgctccctctctggccatgtgacatgcctgctccccctccaccttccaccatgagtggaagcttctcaaggcctcaccagaagaagatgttggtgccatgcttgtatagcctgcagaactagataggatttcaccatgttgcccaggctggtctcgaactcctgggctcaagcgatctgcccacctaggcctcccaaagtactgggattacaggcgttcactgcccggctgagaaggaggttcttccgcgagagaaggctatgcagaggggaggatctcaggcctgaagggtctagaagcccactggaaggtcttccagacacttgggacatctagctcctgaagagtagaaagaggagctgctgccgaagaggtaagacagagagaggggcacaggatgtggagtccgcccctctgtcctcagggagtttctttggagagagaagagaatgaggctgggctcagaggaagttcctcccactccgtggagctcaaactagtccagtggtggcagctgccctggctggaataaagcatcgacccacagtggcctcacatctctgaggcctttgcaggttaagagcctcctgccatgataccccaccacagtggaacccgttgttgggccagcgtgttctcagggactgggacacgggccaaatactacagccaccaaggccatagcctgtgctctctctgagccgagggaagaacaccctctccggccctgtagttccatttttgtttctttgtttcatttcctctgctgtcttctccagagctctgcgctgcagctgagagactgggtatctcctagagattcctgacccccacgcaccagccccaccaccagagggcgcccgagcacagagacgccgcaaccggagcaggagccctaaacctgtcagggtgggtcagccctggagtaatcctggagggagggtctgggagagggccccagtgtggggacctgcccggctcaaccaaatgtagtttgttaccttggtggccatctgcagccaggtggccctgccacactgctaaaccagaggatttaggaagcagacagggattcgggaagtggctgaagagcactggaaagacaaactcctgcggtgcaagagtcctggaccaggagtgcggagaactgggggcgctcctctccctgttaccctaggcttgttttctcatatgtaaacaaaggcaggggcggggagaagggaaggactcgatggtacccaggtgcccttcaggctctaaccaaagacatgccccaccctcctggaggaaccctgcggggcgcagagaacggctggagacatagcggccgcggtttcccccgcagcgcaggagcgccaggcactgcgctgagtcaactgcggtcagggagtcacctgcccgcgacagcgagcgcgcacccacactctcgaccaacccaggctggcctccagcccactctcgggtcccaacgcccaaacatctcgcattgctggctccgacatctccccacccgccgccaggaatcagcgcagagggagatgcgtcttctgtggcgggtgcggggcgaggaccctgaccccagctctgggaagtgaactgtacggaggagagagctctccatcgtcagctctgcctccgcccctcctgcggcttcacttcagcccggatgccggcgatggggaatagaccagggacgcaggagcacactggaccgggggtggatccacttggggaggcaggcaaccgacccgcgcaccggtggcgcaacctcgttccggtgaccgaagcttgtggaggtagctccactcccggctccctggtccgacatccccctgccacctcctcccccacctccccgagtcttgggagcctggaccccgaaggtgcaaaaagtcaagagcctggaaaactgcactgtgcaaatatattgactttatttgtctcctttcaggagcctcacagacatatccaggtaaaaagatcgttaaataaatgccttcagccatc

SEQ ID NO: 6 - DNA, 1361 bp, Homo sapiens
atggcattcgctgtcatccgagctcagagccgtgtgggcagccgcgggctatataagccgcgagcctggccgccgcggggcagacggcgacagcagcggcggcgagcgcctcggagcgcggcggaacagcgccccccgagccccgtgccccccgacgggtccgcccgcccgcccgccctcccgaggagcgccggcccgggcccgcgagggccgccgccaccccgcagcagatttggatcccccgcccggcgagcccccggctgctgcctcccggggggccccggcgcagcggccgccctcggagagccccggcgccccgccgcccggccccgcagacgccggaggcgccatggccgccaagcccggcgagctgatgggcatctgctccagttaccaggcggtgatgccgcacttcgtgtgcctggccgacgagttcccgcagcccgtgcggcccgccaagctgcccaagggccggggccggctgcggcggccgcgccagtctcgcttcaagacgcagccggtgaccttcgacgagatccaggaggtggaggaggagggggtgtcccccatggaggaggagaaggccaagaagtcgttcctgcagagcctggagtgcctgcgccgcagcacgcagagcctgtcgctgcagcgggagcagctcagcagctgcaaactgaggaacagcctggactccagcgactccgactcggccctgtaaggggcgccgcccgcgggggggacgcgcgcgtccgcggtccgcgcggggaccggcgtgtgaaccccgagagtgcccgcgccctgctcccgggggacccgcaaggacccgggaccgccgctcctcgcgcgctcggactcccgccccgctgcgaaccggtcggtgcgcccctcgccgcgctcgccctggcccgggagcgccgggagcggggccgctttcctcgtccttgtaaatgtttattttttaactcttcccagtgcgaactctgctgtgagtgtgtgcggggaggcgcgcccgcgctgagtcggcggcgggtagccactccatgcccttgtccgatggtttgcaactccgattttgcacaccgctccaccgtgccccccagcgcacacccattcacactcacgccaacactctcgctgaacacttttataattgttaggcgtggccgttgggactttgggcgcagcgcggctgctactgcgtctggaggattgatatttatttttgcattgcgatggctgaaggcatttatttaacgatctttttacctggatatgtctgtgaggctcctgaaaggagacaaataaagtcaatatatttgcacagtgcaaaaaaaaaaaaaaaaaaaaaaaaaaaaaaaaaa

SEQ ID NO: 7 - DNA, 1475 bp, Homo sapiens
gtagtacttgcggttctcacgcaccaagaattcgctcttgagcgcgcgccgcggcccgccgccctcctcggcgcccgccgccagctgctcggggctgctagggcccagctgcgcgtagtgttctatgaagtcgcccagcgagtcgcggaactcggcggccaccgccatggacagcgtgaggcggctcttggaaccgcccgcgcccacctcggcgatcttgaggaagcggcccttggcgttctgcttcacatctaagtagaagcgcttgttctggatgtccagccgcttcgaggccagctcctgcgtctcttgctcgccgccgccgcgggacgcgggctggaacccgcacggcccaccgccgccgccgcgctcgctgccgctgtcgccgtccgccatcttctaggccgccgcctcgccgccaccgcccgccgcgctcgcgcccccgccctccggctcgcgctccggggccccccagcctcgcccgcccggctcgctcacgcgctcccgccgctcatcgccggccgccgccgcccccccatcgcctccgcgccccgcccccgcgacttccgtgcagccctagccacttccggcgcgcgatttcccgtgaagtcactccgtcccgccctccgcggcctcgcggccggaagcgccgcggcggcgtggcgcagaggggtgacgtcgggggccgcgccgggcgggcggcaagctgagagtaggctagtgttactgctcggtttctagccctggggacttgccagccttagcgtgactcagccgcccgctgcgtctgtctacgcgcctcttcccgtgacacttttactttagactccggtgtcgtccacttccctccttctcgcttccggggcgttagctgagcatactcaggccccagcctgctcgcgctggttgtgtgctgggaacagagttccagtctatatactggcgaccggggggagggagatcctttggagcactcggaagcgggttctgaccagcctgtggggttccctagaggaggcatgagctgactctgaagggcaaactgcagttggcctggcaactctggagaagagccacccagcataggagacggcagtgcagaggcacccagggcctgatgctgggagctcagagcagctgggggtgtgtatgcctgaagaatggtcgtagatgacactggagggtggattagatcaaggaaagccatttgcagtgcgggagagttagtgcctaccccagagtctggagccaccgtatgtgatcaggttcacattttggaaagatcactgtgggaacaatatagaaagaagatgatttaggatgaggttagatttgggatccagagatcagttaactgtccccacagttagccatattaaagtggttaaggaaattgactgccaatctttagtttctttatcaataaaatgaataattcctaaaaaaaaaaaaaaaaaaaaaaaaaaaaaaaaaaa

SEQ ID NO: 8 - DNA, 9074 bp, Homo sapiens
ggcggcctagaagatggcggacggcgacagcggcagcgagcgcggcggcggcggtgggccgtgcgggttccagcccgcgtcccgcggcggcggcgagcaagagacgcaggagctggcctcgaagcggctggacatccagaacaagcgcttctacttagatgtgaagcagaacgccaagggccgcttcctcaagatcgccgaggtgggcgcgggcggttccaagagccgcctcacgctgtccatggcggtggccgccgagttccgcgactcgctgggcgacttcatagaacactacgcgcagctgggccctagcagccccgagcagctggcggctggcgccgaggagggcggcgggccgcggcgcgcgctcaagagcgaattcttggtgcgtgagaaccgcaagtactacctggacctcaaggagaaccagcgcggccgcttcctgcgcatccgccaaacggtcaaccgcggcggtggcggcttcggcgcgggccccgggccgggcggcttgcagagcggccagaccatcgcgctgcctgcgcagggcctcatcgagttccgcgacgcgctggcgaagctcatagacgactacggaggcgaggacgacgagctggcaggcggcccgggaggcggcgccgggggcccagggggcggcctgtatggagagctcccggagggcacctccatcaccgtggactccaagcgcttcttcttcgatgtgggctgcaacaaatacggggtgtttctgcgagtgagcgaggtgaagccgtcctaccgcaatgccatcaccgtacccttcaaagcctggggcaagttcggaggcgccttttgccggtatgcggatgagatgaaagaaatccaggaacgacagagggataagctttatgagcgacgtggtgggggcagcggcggcggcgaagagtcagagggtgaggaggtggatgaggattgaaacgggcagcttcccctacaggcctccacccaaccaccatccccttggctagagaattccctttcctgctcatccccagtgagctagtggagagggagcaagggaggccgcagagggaaaaaacaaaacgtaacacagttaagagaaataatcgtaagagaacagtgacggacaacttgagaaaagcagtcaagttccaaggaactgacagcaacctgcaaagaggaaaacagcatctcctcacctgcgtaaaattgtctcagcttctgttgtttctcaactgaggttcgtaaacccatcaggataatccctggagggaatagatccttgcacatccagggcaagaaacatgtccaagttacccagaccattgataacagttgcatttaggttgcacctgggtaatctggcataaaagatctctctaggcctcactgttgcggtgtctatcccttcacctccattgaaatcagcattttggatctaggtcttcatggaatccttgagaagagaggcctttacaattacccagttctgagggttcaggttcacgaaaagaaatgcaacttgggataatcatgaacaggttaaagataagatttcaagaagccatctaagaatacagaaccaaattggatccatttttttaaaaaaatggttttgcatggaacctggaccaaggcaaatgtcttttcttcgcagaattgttttccaggatgccagtggattcagatagcaatgcttggagtagaatccgttactaaaatagtttcaaagttgacaaaaaattttcaaagataaaagcagttttacattgggggttgctgaggtaggcacaagaaaaagtcaggcataaagcacaaggcagactgtttgagtggattggttgctgctcactaaagttgttcccctgatctctaaatatggaggtcattaccaagaaatgctttggtatgaatgagagccagatctccactgtgtgagccagtgaattatggctaattcggctgttacagccactggttggctggattttaaaccataaaacttgaagattacctacaaaagtaacagtgtggctataagcctgagctttaatggatatacatcctcacagaaaagttggaaataaccaaaactgaagtcttaatttaccttcagtttaatctgtggatttgttcaaatactaaagatcctcaggtccagaattccagcatcatttattcttttaaaatttttaagaacttgatccattgtatcagtacctcacaatcagagttggcaaatgatggatgagtgattcaagcagtgcacccggtggaagctgaaatccatctgtgaatggaactgaagtgaacgtgaatatgctgactatatcctggaagcatttttataccatcttgaaatttcaacaaactggcttttgccagttaatccagctgtctttcaagaataaaagttggggttttcaaggatcgcctcttctatattttaaatggattttcagtagaaatgatttttactaatcaagttaatcccaccccatcaaaaggtattcctagaaatgtcatagacctaggtaactttgaattgaatgggagctaacgttctttccaaagttttcaggtattctttgtgtgacaccttctcaaccaggaggcaagtaaccccgcctccacaatcttagtattttttttaaactgcatgcctgccccttatttgagctgcctttttaatttattgcatatcctttttattatcttattttggtattattcaatctatacaatctttttgtatttattgggaaatgagtaatatacaaaaaggttttcatgtatttgtggctgagagggcgggaaataattgtgtacataaaattaggcttttttaaaaaaatagattatgatgcagaatattgttgatcttagattaaaaagtggaagagccacaaacattggtgcccttttcagactatttctctactctcatcatccacagtagaatttttaaacagatttttttaaagcttttcttttaaatttttctccgttgcaaagaatgtttcctaaattgtatgggagcaatagtatttttgatgttttaatgacatccgtatacttgtactgtattttgtactacaaggcagctgtttttcaataatgtcctgctgtatttacctacgtgttttgagtgtctatttctttgctgcggagaacaaattcctaaatagttttagtaaaggagctgagaagctagcattaggtttgcagaaactatttaagtttcaactctgaggcagcaatgaaaatttaagttgcagctattagttgattgctgtaactttttcattttcaaaccatgtacaattcttgtatagaccaacttgttttcttgcttcagtggtggttctgttgctcagctgcagtgagccagttcaattttgcaaaggtgcagtacctctcctttttaaggggttggtttattcttttttctttttgtttggctgaattgcagtaactagccttgcctttctattctgtagaaatgacagggtcttcacaatccttcaccagtggctactaagctataattagctgaatagaaagaatgtggaagtggtctgaggcatatagagtatatgccaagaacactaccatatatggcatcagctttggttaccagagaaattttcttagtcattagaccatgtaacagtaatatatcatatgtaaatctttagatatcaatttgaaaatcctccaaaaaaaggagcaaagaatgcataagctatgtgttggcaaaagtaatttatattaaaattttgacctgcctatgtaagattaagtggtaatgtcatagtggtgggtttttaagtcttaaccaatctctaaggtttgtttctcctgcaagggatggttcatggcctctcttcccactgcaggaagattgcagaaggctgtggattaattgtagcatttcactgatcttcactccagtcactagggacaatagaaacctgcaaaacacagattcattcgtaaatattattaatagcttattaaaggaaatggtctttgttaattccagtcagataatggcattgtacagacatgggaaacaaccaatttttttgtttttcagttgttgctatgaatgattttgagcctttttttttaagcttggcaaacatcccagctaatcaaaatagtcatattcctgagaagtaggaaactaaaacttcttttcatataattgttagaaggtttgtttcccaaactaccatagttacaaaggtgaaaagccaaattttaggaacagaatcaaaagaataaaaatctgtgaagagatcctactactcttccttctatgttttggttttggtttctattgtccctatcatttcagcaagtggaacagcagcaagtttttcagtgcatatgctgcacaagaacaaaatataaatctgtatggcaccaaaaatcaaagtgaaaaccaaaccaaaaacccaaacaccctatgtaactatcggaggcatatacgtggtataaatgactgtagctgtgatacacacatggctacttgtcacatcactttccataattatttactgcaaaatgattgagaggcttttggtgcaggcagccgttaacctcctgcttcctttgttacctctggattactttgcagtaaattgcaggtcttttaagagatttaagcttcagttttctcaaaacaaaacaattatcctgtcttatctgaagatgcagggttgtgggcaaaagaggctggttataataatgccctcatattgagtggtctgtaaacggctgcacacttcaggcactgtagttgctgaagatgctttgttaaatgtgaccttgactggctttacaggggtgtagaatgtaatctacacaaggtgactttgcatctatcttgctcttgaggtggatgaaattgagaagctggagtgtgtaagccatgcacataagtattcttcactgtaaattttgttttcatttttaacccaattatggtactttatccaatgcacaactgatctctcagtagatattcatttgaaaatagtgtggccttgatcagtgagaaagggaaggagaaaagtgacttttttgcttatgtagaaatgactcatttgctgagagtttgtctttctgcagcactcttggtatatagtgatcggtctcctttttgattggggaaagttaatgtttttgaccctggagttaattcagttgagttatcttatatttttaggaagtatcagaattgctctgatgaataacaaagttgactgttttgatgtccaatctcaggttttagaatatagtggtgtaaagtcccactatttttaattcttaaaacaactttaatttcgtacaccctaaaagtcacatgcataaggcctgttcagagagcagagcctccatctttttgctccttttctactttgtacttcacttgaaaaaatatcaagtgactttacattgtatatttccattgtaaccctgacatttctcaaagataaagcactttttgatcatgaaatacatgaaatctttgtgtgatgtggatcatagtttctcaggctcccttagataattgcttatgaatattgttctaactctgtgtaagaagagtagaaatctttgctaatgttagaaggtttgtattattgatccagaatgcattttgctagtttccaatggatgggagagtaaataatgctgcattcacaatttaataagttactttcccttgagccttaaggtaactttttctttcctgtcaactacagcactgaagttatagtaagtgaatgagattatcagttttcagggttggttttagagtactgtaaatcaattagctgtcttcctaaagagttacaactcccattcagtatactggataatgggtgtgtgggtggggctggggagggcgggagatagtttgtagaaaagaaaaaagaaaaaaaaaaaaacacatttaccttaagaaaatacagacaaaaaagaataaagtatgtgtcatatgctccatttgagattccaaatgcggccgggcgtggtggctcacgcctgtaatcccagcactctgggaggctgaggtgggcgggtcagttcaggtcagcctggacaacatggtgaaaccccgtctctactaaaaatactaaaattagccgggcatggtggtgcatgcctgtagtcccagctacttgggaggctgaggcaggagaatggcatgaacccaggaagcggaggttgcagtgagccaagatcgcgccattgcactccagcctgggtaacagaacaagactccgtctcaaaaaataaataaataaattccaaatgtaaggtggaaatggcagattataacttcatttcccccatttttaacttagaaaaaacaaagtccttaattttcaagtattagccccccagaagtttcagcagatagctggactcttgaataatgatttcttttgatatgtctgactttgctatttaaagtctcctttcccagccctcagtcttaaaactcagttttattactgacggatttaacagactcttacttgaaaattgtccaattatcagtactgaaaattagatttggaaaatgaaaaaataataaaatcgcatcaagaaaacttattaccttagagattcccaattgatatctgtacccctgatccaagtaaggcctttatgaatgtattttatatttcctaaagaaatagggaatatgccatcagtgtaatcacctctattcacagtcatttaatttgaaaatctaactcatgtctcttgaattagacattccaattgggataggtagagtcatgttaaaacaatagaatagcaactgccatattggtagtttcactgctcaatatgatttctaataagagcagagctttattggaagctaaaactcatttttctccatagatgattctaacagtttaataaaaaagtataattatactttaagtgtcaacagattttgagtctagaagcaaatttcattttccttgtgttatatctaatatccattgacatgggtaagaaaactaattatgtctttttagtatgatgaaatgtgggcccagagtattggaaagaaagcaatagccctattcagtgtccactaggatggatattgcagaaggcatgagtttgtccttttttcccccttaccttaaataacacttcaatagctaactttttccaaaatatccatttaggccatctcttatttcttaaagccaaatatttcgtccccagtcttaactctgagttgcaagatcaggctggttggaagaatctttatatttagaaataggatggtgagtggtttgtttattctcttgtaaaattcctttttcagagacataactgcagggagagtcccaagagaactgaagaactggattatatcattaaacacatttgtgttgtaggaatttgggagtttcattttgaactgataagatattagacataaacccttgttccagcccacatgtgtcatccctgtgtacaaaagctctctggattctttttttttttttgagacggagtttcgctcttatcgtccaggctggagtgagtgtagtggcttgatctcagctcactgcaacctttgcctcccgggctcaagcgattctcctgcctcagcctcccaagtagctgggattacaagcatgtgccaccatgcccagctaattttttgtatttttagtagagacggggtttcaccatgttggccaggctggtctcgaactcctgacctcaagtgatctgcccgcctcagcttcccaaaatgctggaattacaggcatgagccatcacgcctagcctactctctgaatttctaaaagtcagtaggttgaccaaaaagtctagaaactggctttaagtcagtatgggacgtacttataaagagtccatggttttgcacgtttcggtagacaagtaaatctgagttatttttcaatgacttaccaatatttgaatagtaactaagatcgtcagtgtatctggacttctttttttgaagttctaaaacaattatagtagggatttattattttgggcctccatccagatgtttttccaagatcatttttaaaattcatttgtcttctgtttccagataacatactttccgttctataggaatcttcactgccaatcatagtatctaccagtggctttcttagactattcactccaaagctgggactgatgtcctgccagtagagaatctacagaaataatttgaatgaattaaaaccaaatcttgatagcaggagacagcttcctgatctagatgtacaattagagtttaggttggaaattactttaaaatgtgttttttggggatgtcttcaatctctgtgtaaatacccacatgcttatgcattgtaaaccaagtgtgtattcctgtgtatgaatttgtagaactgatttctgcttcaagagaagctgcacctttaattttataaggtcccctccacctgtaaccctataaatgtctgtaaataaaacactaaaatttgtagtgataggatcaatttgggaatatctgctgagagaccaaaaagttcatttttttaagtaccttggttaaagagtaaagattattcctcttattttttaaagaagaatgcactttaacaaacatagagctgcatgggcaattcaaacaaatctgtgaagtgcagtacccattcagaaatcacacttcctgaaaaccgttcaaaagcagagtccagacgggctgttgatctcactgcctgtaggttgaagctcagattctgatcaattttgagaggagcagggctgcttcaaaagagcaatgtgaatacagtcagaagcttcagactggtctgtaaaaatggcgggtcccgtatttaccactaactagcaaaactgacagaaaaactcacagagaaaaaatgtaagaatccttcctgctggtgtgcactccttacaatagacttttgcaaatggagttttacagtctatatttaaaaaaaattgtatgtttgtaacaaataaagtatgcagaaaagtgaatgacaaaaaaa

SEQ ID NO: 9 - DNA, 2843 bp, Homo sapiens
ttattgtatgtacaaatagagaatggaagaacaaaagaatgaggaaaggaagcaagaaaggaaggaagaaagggtgggagggaggaagaacggaagggagggaaagacaaatttcttctcattctttatccctctttgagatgcctctctgtggatcctgtcacctctactggggagcaaaaagaacacagaagttggagccgacggatcccatgcctagctgggtaattaggcaaatctcagcctctggaaatgcagcttcttctgtaacctacctcagagggctgttgtgaggattgaatggggtaacatttaaactaactggcaaaagcaggttctcaatatattaaaattcacatgaaaaagataaaccaggaagggagttggcaggaagccaggaagcctcctgtgttctctgcaggaggcttatctagttcttcaggatcttgggaacagctagtcttcctctgctgcattctgagagttaggtgatgagtcaccacctcaagaggccatctagttctggccgatctatctgactggatcgtggaccctaggacccagctaacttgttctccagccaatttagaagaatgagtagcaaaaataatatggcaagtatttcttgaagtgccctaaccctctgtccttattagtgtaccctgtcaggagtggcctgccttgactttctgtcaaactggagcagcctgagactaagggcaaatgccctcttggccttggccggccacaacttggatatgcaggtcaggcttattgttagggaagctctgagactcaattaatctgatgcctttttattatgagttcaggtgcagctccatttacaagtttttctttcaaaatcattcagccattaaagctgcataatcttggaaggttttgaagacacatgattggtaaatgtttttccccttcacacattatctctcatgtttattcatgaaaagtgaaccagtcttataatttaaacagtgtcttggggtatctggcctcctgtatttctaagggggaggaaagatgaggctatcccagaagccatttaaaaaggggatttgaggcctgaacgcagagaactcaatcccaggtactaatgaccttgctttggcttgcaagactaagcctgaggcctgtggcacttaagagggttactgggtacccagacaggcaaatgcctgttgattggatggctaaattgtgaagaaagcaaagcttacctgtgcaatgtgtccatgccttcattcctactttctgaatcaacagtgcctgttattgttctgcccaactgttaagatgttccacctgtgcctgctctttggcgcagtactccaggctatagaccaacatttctcaaacctggctgcacattagagtcatctggggatcttttaaaaacccagctgccttggctgcacatctgatcattacgtcagactcttgtagggaggagtatagacatcagcattgattaaaactcctgagataattccaatgtgcagtcaggattgagaacagctggtctacaccagttcctcttgaagtatggtctgtaagtcacaggctgcagaatctccttggatgtttgtgggaaatgcagattgctaggttcaccagccctataggcactgagtctcaaggtgagatctggaaaatgtgttttaataagctccccaggtgattcctaagctttttgcaagtaccgctgcttcttatgatatggccgttcttgcttcccatcagttatccttgtctttcagttcctcaaagatatcaagtttcattcatcctcaggaccactgcatatgatagtcgctctacttggaagacattttcctctttctccttgttattaggcatttggcagacttaacaatgaggctattgtaaccccagaaagtcattgcaaagtgcctgcttagggggttaagagtagacactgcagggccttggaatagtaatggtttactatttaatgagttccctgtcactggaagtcatagagagattgctccaggcatggtgggagaaaagccagtttgacaggcggtggagggataagagggaagacttgagtgacgtggcacaaactgggtcagatctattaaagaggcagccctgggcagctggcaagagatcatgtcaagagacacaatgcaggtgccagcaggaccagaagatcagcttcttagagcgagtgccacctggaagggggaaaccaagggaaggggcacagcaaagggcaaagctgggtggtggaggaagtacccaaacagagggaagagccactctggccacatcaagtgaatactgatgatcgggaaacattcatgggaagttatttcactctctcgagtccagtgtaagtgagaaagtgtaatttaatttaaaagagcctaatttagccttccatcaactgtagatcttttaacgagacctttattcttctaggacagcatttctcaaactttaatgtgcgtaggaatcttattaaaatgcagattctgattcagtacgtctggggtaagggctgagattcagcacttctaaccggctcctagatgcccccggtctgagcactacacttagagcaagcaggtttgaacattgcgacttataacagtggacccatggtgtagacaagtccagaaattactgctgaatcaacagtttttatgttagcttccgtataccttcagaattcagtgtttgtgtgcattctaattctctaaatgatgtatattagatttccattaacgaaaatatatgtttaagtggaactctccaattccaacttgccaaataaaataaaaaattcctgtcatttt

SEQ ID NO: 10 - DNA, 3808 bp, Homo sapiens
caggtgatttttggtggggcggggacatgaaaaaaaagttaaaatgtccttataaagacaaaatctttttctttcctggctgatgatttgtcattctagtcacttcctgccttgtgaccacacacccaggcttgacaaagctgttctgcagatcagaaagaaggggttcctggtcatacaccagtactaccaaggacagcttttttcctgcaagatctgttacctaaagcaataaaaaatggccagaggatcagtgtccgatgaggaaatgatggagctcagagaagcttttgccaaagttgatactgatggcaatggatacatcagcttcaatgagttgaatgacttgttcaaggctgcttgcttgcctttgcctgggtatagagtacgagaaattacagaaaacctgatggctacaggtgatctggaccaagatggaaggatcagctttgatgagtttatcaagattttccatggcctaaaaagcacagatgttgccaagacctttagaaaagcaatcaataagaaggaagggatttgtgcaatcggtggtacttcagagcagtctagcgttggcacccaacactcctattcagaggaagaaaagtatgcctttgtcaactggataaacaaagccctggaaaatgatcctgattgtcggcatgtcatcccaatgaacccaaacacgaatgatctctttaatgctgttggagatggcattgtcctttgtaaaatgatcaacctgtcagtgccagacacaattgatgaaagaacaatcaacaaaaagaagctaacccctttcaccattcaggaaaatctgaacttggctctgaactctgcctcagccatcgggtgccatgtggtcaacataggggctgaggacctgaaggaggggaagccttatctggtcctgggacttctgtggcaagtcatcaagattgggttgtttgctgacattgaactcagcagaaatgaagctctgattgctcttttgagagaaggtgagagcctggaggatttgatgaaactctcccctgaagagctcttgctgaggtgggctaattaccacctggaaaatgcaggctgcaacaaaattggcaacttcagtactgacatcaaggactcaaaagcttattaccacctgcttgagcaggtggctccaaaaggagatgaagaaggtgttcctgctgttgttattgacatgtcaggactgcgggagaaggatgacatccagagggcagaatgcatgctgcagcaggcggagaggctgggctgccggcagtttgtcacagccacagatgttgtccgagggaaccccaagttgaacttggcttttattgccaacctctttaacagataccctgccctgcacaaaccagagaaccaggacattgactggggggctcttgaaggtgagacgagagaagagcggacatttaggaactggatgaactccctgggtgttaaccctcgagtcaatcatttgtacagtgacttatcagatgccctggtcatcttccagctctatgaaaagatcaaagttcctgttgactggaacagagtaaacaaaccgccataccccaaactgggaggcaatatgaagaagcttgagaattgtaactacgcggtagaattggggaagaatcaagcgaagttctccctggttggcatcggtggacaagatctcaatgaaggaaaccgcactctcacactggccttgatttggcagctaatgagaaggtatacactgaatatcctcgaagaaattggtggtggccagaaggtcaatgatgacattattgtcaactgggtgaatgaaacattgagggaagcaaagaaaagttcatccatctctagtttcaaggacccgaagattagtacaagtctgcctgttctggacctcatcgatgccatccaaccaggttccattaactatgaccttctgaagacagaaaatctgaatgatgatgagaaactcaacaatgcaaaatatgccatctctatggcccgaaaaattggagcaagagtgtatgccctgccagaagacctggttgaagtgaaccccaaaatggtcatgaccgtgtttgcctgcctcatggggaaaggaatgaagagggtgtgaggcccaatggggctgggtgggaggcggtgcactcactcctgactgcccggcacagatgctccagggatgattcaagccattccaaagttcaacttggtgacactctataagattccaaaaagcacatattagtgcagccaagtagcctctcctgtatttaacaaaaagtgcttcattctttgcaggaggcccaacctcctatatataggtttctattcttgatttatttgcttcttcgaaaatctagaggaaaagaaagaagttattttccaggtacccttctcgcttttgccattagccaaggatagaagctgcagtggtattaattttgatataatctttcaaaccagcttcatgtggcttcccttttctttgttcaagatgagggccaggaggggaaacatcacacctgccctaaaccctgttcctggaggtcagcatttgatctgttgcaagcccctctttctgtcccctcttcctaccctgcctcccttgactttgctcctcacacttttggaaccatgccttccgggggggcccatctcttctggccgtccttgtctctgggccacttggagtgtgtgataaatcagtcaagctgttgaagtctcaggagtctctggtagcctgcagaagtaagcctcatcatcagagcctttcctcaaaactggagtcccaaatgtcatcaggttttgttttttttcagccactaagaacccctctgcttttaactctagaatttgggcttggaccagatctaacatcttgaatactctgccctctagagccttcagccttaatggaaggttggatccaaggagggtgtaatggagcatcaagccactcggcggcagcatggagctataactaagcatcctttagggttctgcctctccaggcatttagccctcacattagatctagttactgtggtatggctaatacctgtcaacatttggaggcaatcctaccttgcttttgcttctagagcttagcatatctgattgttgtcaggccatattatcaatgtttacttttttggtactataaaagctttctgccacccctaaactccaggggggacaatatgtgccaatcaatagcacccctactcacatacacacacacctagccagctgtcaagggcagaatgaatctatgctggataagaaatggtggaactgcgttatgaagagctaatttactggacaaagaattccaaagcaaaaccagaacagtatgaatttgagcaggtctcataggttgagcaatttccccctaaaccaactgaaggctaaaaagcaacaggccattgtgaaccaatgcaagacgccctctatcatggtgaaaagctccatcaatgaggtatcttctttagtggtggtatgtaatggaacttagccatttttcaaagcaattgaaatgcattgctctggatctgttccttggcagtggactcagaaagccaacatgtggctcctcccagcccataaccagtatttttgctgcttctgaatacaaattggttggttttgacttcagattgaacttactgtagcctcagatgatttcccccctccgcctccaggaagaaagaatgttactgccttaataaaaaatgaaaagagaatgatgctcaaaatctttccaaataaaatgttccctataaaaaaaaaaaaaaaaaaa

SEQ ID NO: 11 - DNA, 2409 bp, Homo sapiens
tattctggagttctaatatattttcatggtttttaaatgtttagttctttaatcaagttggaatttttaaaatatatatgaagtaggatgctaatttaaaacttttcctccaaatggatatccatccagttgtctcaacatcatgtatagaaaaaacaaattgaaaatgctatcttatcatattatttactaaatttacatgcatatacatatacatacataacatatatctgagtttgagtctgaattcactactgtttcaattgctgtagttggtcaattctagtatgacagagttttaattgctacagttttggaatgattatggggttacttttgagacagggtctggctatcttgcccagactggtctcaaacttaccggctcaagcgatcctcccatctcagcctcttaaagtgttgggattataggtatgaaccactgcacctggccctggattgcattttgactgtggccatagaagtttccactcagatcttcaagagaacctactctggggactacaagtgactgacagccagctgccacccctttggaactaccatgcattcatgtcaatgccacatatctctggagtcgcccccagctagtggctgagcacagcaggagtactaaacttgttctttcttgcccagtgcagaactcctctaacgggcaactttggcttggggaacttctcatcagcctggatgagactatcttagaactacagtgtagccgaggctctttctacccaatatttccttctcttctttttacagaagtcagacctgcaccacagtctgaagtctctctgccaatccctgagccttcccactttatccttcacaaacatttcccccaataaatatcttgcatgtctaatcccttcttggcatctgcttctctgaggaccagaacagacaggtggtttaccaagtggtgcaagaaaacagatagtaagatgggttttgggcaatggcttacttggtccctggcacaaagcagccatctgagtcctgatttagccagcagtgacctaggaaaacatcccagagaagggaaatcctcttgctcgtgcaatttaggcatttaaaagatatggggtatacgatgcctacaaagaaagcagagctggccaactgttactaagacaagtaatgccttgcagatggataatgagaaaccgagagcacttaaacagttaaaggttaagtgtgggagccgggggcttctctggagcttaacaaacttttatttcctgcagcggaagagcagaaatgggattggatggtcacagagctctaaagatatttgaatgctcagcctagggagatccagtatgtagtcagggccctggttggggaaaccagcctgaagctttgaaacatggaatgggtgcccctgaggattttggctctgtagatacctctggacactcaagagcttacagaactggctcatacttcctagtaagagcaagcacttccctcattacagaaagacctcttccataattgttctcagtttttctcctggctgccaagccatggtaactggggttaacatctcagcataatctgcctaggaactgctggccctgataagaaaggaaagattacacaccgaaggaattgaaataagttaccatgactatcaggagccagagaagtatacttaggattggattttaagggtgttcaattaagggggctggaatttaagacaacataagcaagacttcatggacttggggtacttttttgggacagatttaacctcagcaagtagtcccgaggttagcttttcaaagcctggaaaaggcgatggctttgaaaaagtggaaatgaattcacaccatggcagcttttaacttgaggagaagatagctatttattttggcatatttatgtggttactgctcacttactagtcgcaattgttttgtaaccactttttatttattttttgagatagtctcattctgttttctagactgtgctacagcctgggcgacacagtgagactccgtctcaaaaaaaagaaaaaaaaaaaaatatcatattgtattggccatcccttgtttcatattcgttaataaactttgctactttgctttctaatttcggtgaagaacaagcacaagattaaggagtaagactatgtgaaactgggttataaattgaaaggctgttggctgttgtatcttgtaaacaattcaacgcaatgccaagtgaaaatgaagttgttagtccctctatctagaattctttcccaaatagctgcatatctttttcctctgcttcatttaagtctgtgctgaaatgttatctactcagagaagacattctggatcatttcatattaaatagtaacttttatcactccaaaaaaaaaaaaaaaaaaaa

SEQ ID NO: 12 - DNA, 1338 bp, Homo sapiens
gcccctgctaggacagcccgtgcgagcctgctggaggaggaagagaaaggcagagagagtcgggttacaagatggcggatctgtagtagttaccgcggcggcgggagagcaagcgagccctggggggcaaagagacgggagagtgggtgtatgcgcgggtgaagtgagaggtaacggggctccgggcggagaggcctcagtggctcttgtcaccccttctcgcggctgaacctttggagccatggtgaattcgggcctctccgaagccgccgccgccgccaccgccactactgcctttaccgtctcctaagagtgaggagcgcggacgaggtaagcgaggaggcggcggctagagcggtggagacagcagccaccatgtcggatacgcggcggcgagtgaaggtctataccctgaacgaagaccggcaatgggacgaccgaggcaccgggcacgtctcctccacttacgtggaggagctcaaggggatgtcgctgctggttcgggcagagtccgacggatcactactcttggaatcaaagataaatccaaatactgcatatcagaaacaacaggcaagtatttcagactgaaaaaaagtttttgtactggtgataattatcatttttggattgagccactgtcggtttattctaagatgtatttattagtattatttaactgtagttagccaagtctcttctataccttgtacatgaaaccttttattctgagtcacgtttaaaggaaatgacatagaacaaaattcaaagggatactcttaaagcaaggaaagcctgtttgtggtgtgaaattaaatcctttgactcaagtaaaggtagaggttttgaagtagaagcattaaatcctgctctttaattgttgtgtatttttaatatagtaatcaatggatactttataaaaatgtacattaccaagtatgtaacatcttctgttaatattgtcttatttgggaaagaaaatgtgtattgtattttagcacgcctgattaatctgaaaatggtagcagtgattcaatcttttttattctgtgaatgtttttgtttttattatgaaatatattaaaattgaaaagtacagagaataatcggtgtctactgtaacaccaccagatttaacaatgttaatgttacgccatatttgtttcaaatatttttgtaatattgaacattatggatagagttaaagcttgtttgtatccatcccgttgtttacattctccatcccctacataggtaaccactattctgaagttgatgtgtattctttgtgtacatgcttttataccttttctgcatatgtatgtatccataaataat

SEQ ID NO: 13 - DNA, 719 bp, Homo sapiens
gacaggcttggcaggtactaagccagatggaggcatgaccctgggagggggtggagtgcaaagaggtgcagagcaccccataaaataaggggttgggcatcaccatgccccctgcaggctttgccacccatcagcaccagcaagtcgccccacccagagctctgcaaagaacaccgccagctctttccttccgggtgctcttgctggggggtcaggctggtcccctctgactggcactgcccacgctgggacatgggaggagggccatctgggcactgcaggagcagatcctgggtgcacatggggagacaggtgtgctggcaggaacagcaggcggggctgagctcaggaggcgcctcacagcgggtgcctccagcctggtctgagcacgggatgaagaaccaggcaccgctcatcgtgcatctccacaaggcactgtgtgacttcatgcaagccgctcaacctctctggccccacgtccttgtccacaggggcagagtggactgaatggaggctgaggtccctctcagcttgggtggtgtggcagagggtgaggaggagcccatgggccctggatcaccagcctccctccacccagaatgccccgtccactccactgcccaccaccctctgtgcaattgacaaacgtcctggtgttaacgcctaagcataaagagtcggctttgctgtaaaaaaaaaaaaaaaaaaaaaaaaaaaaa

SEQ ID NO: 14 - DNA, 2985 bp, Homo sapiens
tcgggcacggcgtcctccctccgcagcagccgagccggacctgcctccccgggcgtgctccgccggccccgccgccggcccgcagcgacagacaggcgctccccgcagctccgcacgggacccaggccgccggaccccagcgccggaccaccctctgtccgccccgaggagtttgccgcctgccggagcacctgcgcacagatggagctggaccaccggaccagcggcgggctccacgcctaccccgggccgcggggcgggcaggtggccaagcccaacgtgatcctgcagatcgggaagtgccgggccgagatgctggagcacgtgcggcggacgcaccggcacctgctggccgaggtgtccaagcaggtggagcgcgagctgaaggggctgcaccggtcggtcgggaagctggagagcaacctggacggctacgtgcccacgagcgactcgcagcgctggaagaagtccatcaaggcctgcctgtgccgctgccaggagaccatcgccaacctggagcgctgggtcaagcgcgagatgcacgtgtggcgcgaggtgttctaccgcctggagcgctgggccgaccgcctggagtccacgggcggcaagtacccggtgggcagcgagtcagcccgccacaccgtttccgtgggcgtggggggtcccgagagctactgccacgaggcagacggctacgactacaccgtcagcccctacgccatcaccccgcccccagccgctggcgagctgcccgggcaggagcccgccgaggcccagcagtaccagccgtgggtccccggcgaggacgggcagcccagccccggcgtggacacgcagatcttcgaggaccctcgagagttcctgagccacctagaggagtacttgcggcaggtgggcggctctgaggagtactggctgtcccagatccagaatcacatgaacgggccggccaagaagtggtgggagttcaagcagggctccgtgaagaactgggtggagttcaagaaggagttcctgcagtacagcgagggcacgctgtcccgagaggccatccagcgcgagctggacctgccgcagaagcagggcgagccgctggaccagttcctgtggcgcaagcgggacctgtaccagacgctctacgtggacgcggacgaggaggagatcatccagtacgtggtgggcaccctgcagcccaagctcaagcgtttcctgcgccaccccctgcccaagaccctggagcagctcatccagaggggcatggaggtgcaggatgacctggagcaggcggccgagccggccggcccccacctcccggtggaggatgaggcggagaccctcacgcccgcccccaacagcgagtccgtggccagtgaccggacccagcccgagtagagggcatcccggagcccccagcctgcccactacatccagcctgtggctttgcccaccaggacttttgagctggggctgactcctgcaggggaagccctggtccagctgggtgccccctcgagctccgggcggactcgcacacactcgtgtcatccagatgtgagcaccgcacccagcggcaaagagccctcccccctgcagggctccacccatcaccctccctccgtctgtctttccggcctggaccccaccctccacactctcaggccatcacagaacaccccagcttcctcattctgctacaacacccaggccctctggacatccagaaaaccaagtgtccggatggcaggggccagcggccaccaagctcatgggacacccagagcagaagctagggcagagccaatgctgagggagcctcgacttccggcgccgccgccctctcccggcatccgcagagccagctgacgccctccctgcctcccagggcagctggccagcctcgggcagcgcggccccctcctcccaggggagagtagaagtcgcacacgcagcagagcagacctgatgtcccggtgcttcctggcccctcagctccagtgattcacgcccgcctggagaagaatcagagctcagctcatgactcacccatggcaggcggagggtcccagaggggctgagtcctcaaatccggctgaggcagcagctggcaccatcagagccaggagagtgacaacaggtctcaaggttcccacaaagtctttgctgctgtgctgggcaccacccacccctcaccttgcaggctgcctgcgtgggaggcgaagtcccaggacagcccagaggggggctacagagaggagtcggctgcagcagagggcaggagccccagcttagccctgagcgccagcgcgaggaccagggcctgccactaagcccgccccgctggccgccagctgcccgtccccagagccactgcagcaggagtcgggccctgcctccctcccagcagggaaaccccgcccgctgccaggccatcctctctgccagaggctttcatgagccccaaggctggggccacagctcctacccctgcccagcagccctgagctcagctgcaggaaggacatcccagaagccatggctcctggggcgcttccaggcattctgccctgccccgacaccagaaccctggtgctggtgggccactagcgtctgcagcctaagcaggtgctggctcagggttcatcgttctgccttgtccactgggggaccagccctgcagaccactctgacaagtcttcagcccacaccctgccagccccacagattttatttttgcacataagccataaccaatcctcaaggctggcacaggctttggggaagccctggagcctgtgaagaccctggaaacctcatgaggctgtggccaacccctgccccttgccccacacagaccaggccttaaatgtcggtccaggccctgtgcaccttaccccagagacagactctttttgtaagattttgttaataaaacactgaaacttcaaaaaaaaaaaaaaaaaaaaaaaaaaaaaaaaaaaaaaaaaa

SEQ ID NO: 15 - DNA, 848 bp, Homo sapiens
  misc_feature (1)..(1): n is a, c, g, or t
  misc_feature (33)..(33): n is a, c, g, or t
  misc_feature (39)..(39): n is a, c, g, or t
  misc_feature (92)..(92): n is a, c, g, or t
  misc_feature (207)..(207): n is a, c, g, or t
  misc_feature (217)..(217): n is a, c, g, or t
  misc_feature (228)..(228): n is a, c, g, or t
  misc_feature (267)..(267): n is a, c, g, or t
  misc_feature (314)..(314): n is a, c, g, or t
  misc_feature (420)..(420): n is a, c, g, or t
  misc_feature (449)..(449): n is a, c, g, or t
  misc_feature (460)..(460): n is a, c, g, or t
  misc_feature (477)..(477): n is a, c, g, or t
  misc_feature (490)..(490): n is a, c, g, or t
  misc_feature (499)..(499): n is a, c, g, or t
  misc_feature (540)..(540): n is a, c, g, or t
ngaggaacaaaagtcgttttgtttggtgatgcnccgggnaaggcagtgagctgtttgcttttttacagcgaaggtgagaggcaagttatctntatttgtggacttcaaagaggcgtgttcggaacaagtcccaaagctttggcatgatgtttattagcaaagttcagtgaattgcaaattaatacccacagtggcatttatgaatgngggggggggncttccctcttnttgggtatgttgtacttgtttgtttttggaggttcccantttagttccagcccactgcctgggttggaagtccgagaggaaacgangagttctccgggaggctgacttatacttcctcacggatgcaggagacggggtaagcccttcccgcctccctccgcgggcaggaggctatgctaaccaagcagccancagaagtgcaggagaccaccgtggagtgncctgaaatcancacactccagtgaaganatttgctagaggnggggtgggngacttttcaagacaatggattgtcaggtgttatgcaaatanagaatggagaggagggttcctggcttctgttgtggcgtcttttctatttgacttcatggttgtaagtatttccagatggtgaatcagacaccagacgtaacaatatttccatatttgggtatagcagtagcctgcgtttttaacatttttctgcctctgttatccaaccacaaagcattcttgacagcttcaaatgttgttaaatatagatttaacttctcttcccagagcaggaaattctttggaattccttgtttttcacgcaatctgtccatcatgatttaaaataaaagcacagtggatcatcc

SEQ ID NO: 16 - DNA, 2295 bp, Homo sapiens
agaccgcgagcgagagcgcccccgagcagcgcccgcgccctccgcgccttctccgccgggacctcgagcgaaagacgcccgcccgccgcccagccctcgcctccctgcccaccgggcccaccgcgccgccaccccgaccccgctgcgcacggcctgtccgctgcacaccagcttgttggcgtcttcgtcgccgcgctcgccccgggctactcctgcgcgccacaatgagctcccgcatcgccagggcgctcgccttagtcgtcacccttctccacttgaccaggctggcgctctccacctgccccgctgcctgccactgccccctggaggcgcccaagtgcgcgccgggagtcgggctggtccgggacggctgcggctgctgtaaggtctgcgccaagcagctcaacgaggactgcagcaaaacgcagccctgcgaccacaccaaggggctggaatgcaacttcggcgccagctccaccgctctgaaggggatctgcagagctcagtcagagggcagaccctgtgaatataactccagaatctaccaaaacggggaaagtttccagcccaactgtaaacatcagtgcacatgtattgatggcgccgtgggctgcattcctctgtgtccccaagaactatctctccccaacttgggctgtcccaaccctcggctggtcaaagttaccgggcagtgctgcgaggagtgggtctgtgacgaggatagtatcaaggaccccatggaggaccaggacggcctccttggcaaggagctgggattcgatgcctccgaggtggagttgacgagaaacaatgaattgattgcagttggaaaaggcagctcactgaagcggctccctgtttttggaatggagcctcgcatcctatacaaccctttacaaggccagaaatgtattgttcaaacaacttcatggtcccagtgctcaaagacctgtggaactggtatctccacacgagttaccaatgacaaccctgagtgccgccttgtgaaagaaacccggatttgtgaggtgcggccttgtggacagccagtgtacagcagcctgaaaaagggcaagaaatgcagcaagaccaagaaatcccccgaaccagtcaggtttacttacgctggatgtttgagtgtgaagaaataccggcccaagtactgcggttcctgcgtggacggccgatgctgcacgccccagctgaccaggactgtgaagatgcggttccgctgcgaagatggggagacattttccaagaacgtcatgatgatccagtcctgcaaatgcaactacaactgcccgcatgccaatgaagcagcgtttcccttctacaggctgttcaatgacattcacaaatttagggactaaatgctacctgggtttccagggcacacctagacaaacaagggagaagagtgtcagaatcagaatcatggagaaaatgggcgggggtggtgtgggtgatgggactcattgtagaaaggaagccttgctcattcttgaggagcattaaggtatttcgaaactgccaagggtgctggtgcggatggacactaatgcagccacgattggagaatactttgcttcatagtattggagcacatgttactgcttcattttggagcttgtggagttgatgactttctgttttctgtttgtaaattatttgctaagcatattttctctaggcttttttccttttggggttctacagtcgtaaaagagataataagattagttggacagtttaaagcttttattcgtcctttgacaaaagtaaatgggagggcattccatcccttcctgaagggggacactccatgagtgtctgtgagaggcagctatctgcactctaaactgcaaacagaaatcaggtgttttaagactgaatgttttatttatcaaaatgtagcttttggggagggaggggaaatgtaatactggaataatttgtaaatgattttaattttatattcagtgaaaagattttatttatggaattaaccatttaataaagaaatatttacctaatatctgagtgtatgccattcggtatttttagaggtgctccaaagtcattaggaacaacctagctcacgtactcaattattcaaacaggacttattgggatacagcagtgaattaagctattaaaataagataatgattgcttttataccttcagtagagaaaagtctttgcatataaagtaatgtttaaaaaacatgtattgaacacgacattgtatgaagcacaataaagattctgaagctaaatttgtgatttaagaaaa

SEQ ID NO: 17 - DNA, 340 bp, Homo sapiens
atgggcggagggaagctcatcagtggggccacgagctgagtgcgtcctgtcactccactcccatgtcccttgggaaggtctgagactagggccagaggcggccctaacagggctctccctgagcttcagggaggtgagttcccagagaacggggctccgcgcgaggtcagactgggcaggagatgccgtggaccccgcccttcggggaggggcccggcggatgcctcctttgccggagcttggaacagactcacggccagcgaagtgagttcaatggctgaggtgaggtaccccgcaggggacctcataacccaattcagaccactctcctccgcccatt

SEQ ID NO: 18 - DNA, 2037 bp, Homo sapiens
catcttctaaattgagcctccggtcatactagttttgtgcttggaaccttgcttcaagaagatccctaagctgtagaacattttaacgttgatgccacaacgcagattgatgccttgtagatggagcttgcagatggagccccgtgacctctcacctacccacctgtttgcctgccttcttgtgcgtttctcggagaagttcttagcctgatgaaataacttggggcgttgaagagctgtttaattttaaatgccttagactggggatatattagaggaagcagattgtcaaattaagggtgtcattgtgttgtgctaaacgctgggagggtacaagttggccattcctaaatctgtgtgtgagaaatggcaggtctagtttgggcattgtgattgcattgcagattactaggagaagggaatggtgggtacaccggtagtgctcttttgttcttgcttcgtttttttaaacttgaactttacttcgttagatttcataatactttcttggaattctctggggctttggggaatttagtgcgtgggtgagccaagaaaatactaattaataatagtaagttgttagtgttggttaagttgttgcttggaagtgagaagttgcttagaaactttccaaagtgcttagaactttaagtgcaaacagacaaactaacaaacaaaaattgttttgctttgctacaaggtggggaagactgaagaagtgttaactgaaaacaggtgacacagagtcaccagttttccgagaaccaaagggaggggtgtgtgatgccatctcacaggcaggggaaatgtctttaccagcttcctcctggtggccaagacagcctgtttcagagggttgttttgtttggggtgtgggtgttatcaagtgaattagtcacttgaaagatgggcgtcagacttgcatacgcagcagatcagcatccttcgctgccccttagcaacttaggtggttgatttgaaactgtgaaggtgtgattttttcaggagctggaagtcttagaaaagccttgtaaatgcctatattgtgggcttttaacgtatttaagggaccacttaagacgagattagatgggctcttctggatttgttcctcatttgtcacaggtgtcttgtgattgaaaatcatgagcgaagtgaaattgcattgaatttcaagggaatttagtatgtaaatcgtgccttagaaacacatctgttgtcttttctgtgtttggtcgatattaataatggcaaaatttttgcctatctagtatcttcaaattgtagtctttgtaacaaccaaataaccttttgtggtcactgtaaaattaatatttggtagacagaatccatgtacctttgctaaggttagaatgaataatttattgtatttttaatttgaatgtttgtgctttttaaatgagccaagactagaggggaaactatcacctaaaatcagtttggaaaacaagacctaaaaagggaaggggatggggattgtggggagagagtgggcgaggtgcctttactacatgtgtgatctgaaaaccctgcttggttctgagctgcgtctattgaattggtaaagtaataccaatggctttttatcatttccttcttccctttaagtttcacttgaaattttaaaaatcatggttatttttatcgttgggatctttctgtcttctgggttccattttttaaatgtttaaaaatatgttgacatggtagttcagttcttaaccaatgacttggggatgatgcaaacaattactgtcgttgggatttagagtgtattagtcacgcatgtatggggaagtagtctcgggtatgctgttgtgaaattgaaactgtaaaagtagatggttgaaagtactggtatgttgctctgtatggtaagaactaattctgttacgtcatgtacataattactaatcacttttcttcccctttacagcacaaataaagtttgagttctaaactcaaaaaaaaaaaaaaaaaaaaaaaaaaaaaagaaaaaaaaaaaaaaaaaaa

SEQ ID NO: 19 - DNA, 1338 bp, Homo sapiens
cggatcgcctctcggatgccgcttgcctggaagctgcgttaggagcgagcggcggcggtggcggcggtggcggcggcggcggcagctcgggagtgctatgaccggcaaactcgccgagaagctgccggtgaccatgagcagtttgctaaaccaactgcctgacaatctgtaccccgaggagatccccagcgcgctcaacctcttctccggcagcagcgactcggtagtccattacaatcagatggctacagagaatgtaatggacatcggtctgaccaacgagaagcccaacccggaactctcttactccggctccttccagccagcccccggcaacaagaccgtgacctacttgggaaagttcgccttcgactccccttccaactggtgccaggacaacatcattagcctcatgagcgccggcatcttgggggtgcccccggcttcaggggcgctcagcacgcagacgtccacggccagcatggtgcagccaccgcagggtgacgtggaggccatgtatcccgcgctacccccctactccaactgcggcgacctctactcagagcccgtgtctttccacgacccccagggcaatcccgggctcgcctattccccccaggattaccaatcggccaagccggcgttggacagcaatctcttccccatgattcctgactacaacctctaccaccaccccaacgacatgggctccattccggagcacaagcccttccagggcatggaccccatccgggtcaacccgccccctattacccctctggagaccatcaaggcattcaaagacaagcagatccacccgggctttggcagcctgccccagccgccgctcaccctcaagcccatccggccccgcaagtaccccaaccggcctagcaagacaccgctccacgaacggccccacgcgtgcccggccgagggctgcgaccgccgtttcagccgttcggacgagctgacccggcacctgcgcatccacacgggccacaagcccttccagtgccggatctgcatgcggagcttcagccgcagcgaccacctcaccactcacatccgcactcatacgggcgagaagccctttgcctgcgagttctgcgggcgcaagtttgcgcgcagcgacgagcgcaagcgccacgccaagatccacctcaagcaaaaggagaagaaggcggagaagggcggtgcaccctctgcatcctcggcgccccccgtgtcgctggcccccgtggtcaccacctgcgcctgaggatcgggcccccagatccccacttttcccctccagtgcctcccggctgctagcctgaaagcagcgggaaagccagc

SEQ ID NO: 20 - DNA, 1635 bp, Homo sapiens
tgctcttcccgcgctgtcccgccgctcctgaccccttcagactgtcccgagaagtctaggaaggcaccggagaccctcggcacaaggcactgaacctggagcgcctgaggggaactgacgcttgcttggcacctttgggaagactgcgaaccagctcaggtcccaggggagcgtgtccgtgtctttttcggccgcattgatgcacccgcccaagtcatttcctcagtcttctgcttcctagaagcaaaacaaggaataacattatgatacctttaaaaagctgaggcgtgaaactgctcctgcaacttggaagcgcagggcacttgaactttattccctaaatgctcgaaaaagaaatctttctggtggcagcttgctactcaagatgagtgactctgaccacaaccggctgaaattaacttgaggccctagatcatttgctatgacactaattccacgttgttcctcaattatgtctccattacctcaaaggctgaagtcgtcctggctgcacggagcagcccagtcttgtctctgtaaataccgcagatttgaaaaatagtcctattggaacccgtcagagggatgtaatacaagggactgtgatggctcttgaacggagccagagactcttcctgaaggactggactacctccccgcctaggaaggttgggtaacccaaagttgccacatttctgggctgcacaccaaggagaggccccggccactccacacaggaaatgcacgtctgggcacctggagccgcaggacctgagccactaactgtccctggatcccttctgcttcagaggcaaggaagactgccggagtcattgcttgcctgctggcttcaccagcgctttctgtttttccagttctcctcaccactcttcctggcgcctttctctgcctcccgcacccctgcctcctctaaaacccacagcatcatccctcagaacagctggaagcggacagctgatccttctccttgctggtcccccagcccagaacacagcctgggacatacgtgcggagccacagtgtctgttggtctcacctgagagctgcttgtcactcccacaccctctttcaaggagaagctgtcagcattgtccttctgcagcattcaacaggggccaggcattgggttagggctgaaaacactgatggagagccatcatctttgccttctaaagggcttggactgtgataagaaggtggacacttgaagtggctgcaggaccggcattccagtgctggaggggtgtttagtcctccatcagctcagagaagtaaaggcacatttattggcagggaaaacccaaggagggcttctcaggagagacacacatttgactctgggggaaagtggtccatagtaccatgtacaaaggtttaatttgctaatacattttttcaaatgccattttgatatttttaactcataaagagacaacactttccaaacctgctgccagcaatgaatctacattgtgtctgtaaagccagaacaggtatttgatggacagctgaaacaatctttataatctaagatgctaatcaatgatattaacatgttgctcaattatttaaggttagtagagtcacagtcgaatctaagatttatattaaaac

SEQ ID NO: 21 - DNA, 8110 bp, Homo sapiens
gatcagagtgggccactgccagccaacggcccccggggctcaggcggggagcagctctgtggtgtgggattgaggcgttttccaagagtgggttttcacgtttctaagatttcccaagcagacagcccgtgctgctccgatttctcgaacaaaaaagcaaaacgtgtggctgtcttgggagcaagtcgcaggactgcaagcagttgggggagaaagtccgccattttgccacttctcaaccgtccctgcaaggctggggctcagttgcgtaatggaaagtaaagccctgaactatcacactttaatcttccttcaaaaggtggtaaactatacctactgtccctcaagagaacacaagaagtgctttaagaggcggcggaaggtgatcgaattccggtgatgcgagttgttctccgtctataaatacgcctcgcccgagctgtgcggtaggcattgaggcagccagcgcaggggcttctgctgagggggcaggcggagcttgaggaaaccgcagataagtttttttctctttgaaagatagagattaatacaactacttaaaaaatatagtcaataggttactaagatattgcttagcgttaagtttttaacgtaattttaatagcttaagattttaagagaaaatatgaagacttagaagagtagcatgaggaaggaaaagataaaaggtttctaaaacatgacggaggttgagatgaagcttcttcatggagtaaaaaatgtatttaaaagaaaattgagagaaaggactacagagccccgaattaataccaatagaagggcaatgcttttagattaaaatgaaggtgacttaaacagcttaaagtttagtttaaaagttgtaggtgattaaaataatttgaaggcgatcttttaaaaagagattaaaccgaaggtgattaaaagaccttgaaatccatgacgcagggagaattgcgtcatttaaagcctagttaacgcatttactaaacgcagacgaaaatggaaagattaattgggagtggtaggatgaaacaatttggagaagatagaagtttgaagtggaaaactggaagacagaagtacgggaaggcgaagaaaagaatagagaagatagggaaattagaagataaaaacatacttttagaagaaaaaagataaatttaaacctgaaaagtaggaagcagaagagaaaagacaagctaggaaacaaaaagctaagggcaaaatgtacaaacttagaagaaaattggaagatagaaacaagatagaaaatgaaaatattgtcaagagtttcagatagaaaatgaaaaacaagctaagacaagtattggagaagtatagaagatagaaaaatataaagccaaaaattggataaaatagcactgaaaaaatgaggaaattattggtaaccaatttattttaaaagcccatcaatttaatttctggtggtgcagaagttagaaggtaaagcttgagaagatgagggtgtttacgtagaccagaaccaatttagaagaatacttgaagctagaaggggaagttggttaaaaatcacatcaaaaagctactaaaaggactggtgtaatttaaaaaaaactaaggcagaaggcttttggaagagttagaagaatttggaaggccttaaatatagtagcttagtttgaaaaatgtgaaggactttcgtaacggaagtaattcaagatcaagagtaattaccaacttaatgtttttgcattggactttgagttaagattattttttaaatcctgaggactagcattaattgacagctgacccaggtgctacacagaagtggattcagtgaatctaggaagacagcagcagacaggattccaggaaccagtgtttgatgaagctaggactgaggagcaagcgagcaagcagcagttcgtggtgaagataggaaaagagtccaggagccagtgcgatttggtgaaggaagctaggaagaaggaaggagcgctaacgatttggtggtgaagctaggaaaaaggattccaggaaggagcgagtgcaatttggtgatgaaggtagcaggcggcttggcttggcaaccacacggaggaggcgagcaggcgttgtgcgtagaggatcctagaccagcatgccagtgtgccaaggccacagggaaagcgagtggttggtaaaaatccgtgaggtcggcaatatgttgtttttctggaacttacttatggtaaccttttatttattttctaatataatgggggagtttcgtactgaggtgtaaagggatttatatggggacgtaggccgatttccgggtgttgtaggtttctctttttcaggcttatactcatgaatcttgtctgaagcttttgagggcagactgccaagtcctggagaaatagtagatggcaagtttgtgggttttttttttttacacgaatttgaggaaaaccaaatgaatttgatagccaaattgagacaatttcagcaaatctgtaagcagtttgtatgtttagttggggtaatgaagtatttcagttttgtgaatagatgacctgtttttacttcctcaccctgaattcgttttgtaaatgtagagtttggatgtgtaactgaggcgggggggagttttcagtatttttttttgtgggggtgggggcaaaatatgttttcagttctttttcccttaggtctgtctagaatcctaaaggcaaatgactcaaggtgtaacagaaaacaagaaaatccaatatcaggataatcagaccaccacaggtttacagtttatagaaactagagcagttctcacgttgaggtctgtggaagagatgtccattggagaaatggctggtagttactcttttttccccccacccccttaatcagactttaaaagtgcttaaccccttaaacttgttattttttacttgaagcattttgggatggtcttaacagggaagagagagggtgggggagaaaatgtttttttctaagattttccacagatgctatagtactattgacaaactgggttagagaaggagtgtaccgctgtgctgttggcacgaacaccttcagggactggagctgcttttatccttggaagagtattcccagttgaagctgaaaagtacagcacagtgcagctttggttcatattcagtcatctcaggagaacttcagaagagcttgagtaggccaaatgttgaagttaagttttccaataatgtgacttcttaaaagttttattaaaggggaggggcaaatattggcaattagttggcagtggcgtgttacggtgggattggtggggtgggtttaggtaattgtttagtttatgattgcagataaactcatgccagagaacttaaagtcttagaatggaaaaagtaaagaaatatcaacttccaagttggcaagtaactcccaatgatttagtttttttccccccagtttgaattgggaagctgggggaagttaaatatgagccactgggtgtaccagtgcattaatttgggcaaggaaagtgtcataatttgatactgtatctgttttccttcaaagtatagagcttttggggaaggaaagtattgaactgggggttggtctggcctactgggctgacattaactacaattatgggaaatgcaaaagttgtttggatatggtagtgtgtggttctcttttggaatttttttcaggtgatttaataataatttaaaactactatagaaactgcagagcaaaggaagtggcttaatgatcctgaagggatttcttctgatggtagcttttgtattatcaaacttttttcagataacatcttctgagtcataaccagcctggcagtatgatggcctagatgcagagaaaacagctccttggtgaattgataagtaaaggcagaaaagattatatgtcatacctccattggggaataagcataaccctgagattcttactactgatgagaacattatctgcatatgccaaaaaattttaagcaaatgaaagctaccaatttaaagttacggaatctaccattttaaagttaattgcttgtcaagctataaccacaaaaataatgaattgatgagaaatacaatgaagaggcaatgtccatctcaaaatactgcttttacaaaagcagaataaaagcgaaaagaaatgaaaatgttacactacattaatcctggaataaaagaagccgaaataaatgagagatgagttgggatcaagtggattgaggaggctgtgctgtgtgccaatgtttcgtttgcctcagacaggtatctcttcgttatcagaagagttgcttcatttcatctgggagcagaaaacagcaggcagctgttaacagataagtttaacttgcatctgcagtattgcatgttagggataagtgcttatttttaagagctgtggagttcttaaatatcaaccatggcactttctcctgaccccttccctaggggatttcaggattgagaaatttttccatcgagcctttttaaaattgtaggacttgttcctgtgggcttcagtgatgggatagtacacttcactcagaggcatttgcatctttaaataatttcttaaaagcctctaaagtgatcagtgccttgatgccaactaaggaaatttgtttagcattgaatctctgaaggctctatgaaaggaatagcatgatgtgctgttagaatcagatgttactgctaaaatttacatgttgtgatgtaaattgtgtagaaaaccattaaatcattcaaaataataaactatttttattagagaatgtatacttttagaaagctgtctccttatttaaataaaatagtgtttgtctgtagttcagtgttggggcaatcttgggggggattcttctctaatctttcagaaactttgtctgcgaacactctttaatggaccagatcaggatttgagcggaagaacgaatgtaactttaaggcaggaaagacaaattttattcttcataaagtgatgagcatataataattccaggcacatggcaatagaggccctctaaataaggaataaataacctcttagacaggtgggagattatgatcagagtaaaaggtaattacacattttatttccagaaagtcaggggtctataaattgacagtgattagagtaatactttttcacatttccaaagtttgcatgttaactttaaatgcttacaatcttagagtggtaggcaatgttttacactattgaccttatatagggaagggagggggtgcctgtggggttttaaagaattttcctttgcagaggcatttcatccttcatgaagccattcaggattttgaattgcatatgagtgcttggctcttccttctgttctagtgagtgtatgagaccttgcagtgagtttatcagcatactcaaaatttttttcctggaatttggagggatgggaggagggggtggggcttacttgttgtagctttttttttttttacagacttcacagagaatgcagttgtcttgacttcaggtctgtctgttctgttggcaagtaaatgcagtactgttctgatcccgctgctattagaatgcattgtgaaacgactggagtatgattaaaagttgtgttccccaatgcttggagtagtgattgttgaaggaaaaaatccagctgagtgataaaggctgagtgttgaggaaatttctgcagttttaagcagtcgtatttgtgattgaagctgagtacattttgctggtgtatttttaggtaaaatgctttttgttcatttctggtggtgggaggggactgaagcctttagtcttttccagatgcaaccttaaaatcagtgacaagaaacattccaaacaagcaacagtcttcaagaaattaaactggcaagtggaaatgtttaaacagttcagtgatctttagtgcattgtttatgtgtgggtttctctctcccctcccttggtcttaattcttacatgcaggaacactcagcagacacacgtatgcgaagggccagagaagccagacccagtaagaaaaaatagcctatttactttaaataaaccaaacattccattttaaatgtggggattgggaaccactagttctttcagatggtattcttcagactatagaaggagcttccagttgaattcaccagtggacaaaatgaggaaaacaggtgaacaagctttttctgtatttacatacaaagtcagatcagttatgggacaatagtattgaatagatttcagctttatgctggagtaactggcatgtgagcaaactgtgttggcgtgggggtggaggggtgaggtgggcgctaagcctttttttaagatttttcaggtacccctcactaaaggcaccgaaggcttaaagtaggacaaccatggagccttcctgtggcaggagagacaacaaagcgctattatcctaaggtcaagagaagtgtcagcctcacctgatttttattagtaatgaggacttgcctcaactccctctttctggagtgaagcatccgaaggaatgcttgaagtacccctgggcttctcttaacatttaagcaagctgtttttatagcagctcttaataataaagcccaaatctcaagcggtgcttgaaggggagggaaagggggaaagcgggcaaccacttttccctagcttttccagaagcctgttaaaagcaaggtctccccacaagcaacttctctgccacatcgccaccccgtgccttttgatctagcacagacccttcacccctcacctcgatgcagccagtagcttggatccttgtgggcatgatccataatcggtttcaaggtaacgatggtgtcgaggtctttggtgggttgaactatgttagaaaaggccattaatttgcctgcaaattgttaacagaagggtattaaaaccacagctaagtagctctattataatacttatccagtgactaaaaccaacttaaaccagtaagtggagaaataacatgttcaagaactgtaatgctgggtgggaacatgtaacttgtagactggagaagataggcatttgagtggctgagagggcttttgggtgggaatgcaaaaattctctgctaagactttttcaggtgaacataacagacttggccaagctagcatcttagcggaagctgatctccaatgctcttcagtagggtcatgaaggtttttcttttcctgagaaaacaacacgtattgttttctcaggttttgctttttggcctttttctagcttaaaaaaaaaaaaagcaaaagatgctggtggttggcactcctggtttccaggacggggttcaaatccctgcggtgtctttgctttgactactaatctgtcttcaggactctttctgtatttctccttttctctgcaggtgctagttcttggagttttggggaggtgggaggtaacagcacaatatctttgaactatatacatccttgatgtataatttgtcaggagcttgacttgattgtatattcatatttacacgagaacctaatataactgccttgtctttttcaggtaatagcctgcagctggtgttttgagaagccctactgctgaaaacttaacaattttgtgtaataaaaatggagaagctctaaattgttgtggttcttttggaataaaaaaatcttgattgggaaaaaagatgggtgttctgtgggcttgttctgttaaatctgtggtctataaacacagcacccataattacagcataatcttcaagtagggtacggactttgggggattggtgcgagggtagtgggtgagtggcctactaaaaagcccagtaacccccacaggaaaatagggaacttctttttaagtagcctcctttccactatttagtaattggctgtgagctgggctgggggagaaatggggcggggtgtgtgtgtcattggaaagctctcttttttgtttttttgagacagtctcactttgtcccccaggctggagtgtagtggcatgatctctgcaaactgcaacctccacttgtggggtccaagtggttgtcctgcttcaccctccctgtagctgggactacaggtgcacaccaccacgcctggctaatttttgtatt

SEQ ID NO: 22 - DNA, 1927 bp, Homo sapiens
actttaagtcttttccttttggtcttggaggaacagtgctcagcttttgatctctgtgattctgatagagaagctgtttctggaattacaaagtttgcaaacttgacagcaaaatttctggaaagccttttctatgaacctcggacaagtctaatctggagctgatcactctaacatgcacctgtgtggctgcgactctcttctggctcctattaaccctctttatccgaaaaatgaaaaggtcttcttctgaaataaagactgactacctatcaattataatggacccagatgaagttcctttggatgagcagtgtgagcggctcccttatgatgccagcaagtgggagtttgcccgggagagacttaaactgggcaaatcacttggaagaggggcttttggaaaagtggttcaagcatcagcatttggcattaagaaatcacctacgtgccggactgtggctgtgaaaatgctgaaagagggggccacggccagcgagtacaaagctctgatgactgagctaaaaatcttgacccacattggccaccatctgaacgtggttaacctgctgggagcctgcaccaagcaaggagggcctctgatggtgattgttgaatactgcaaatatggaaatctctccaactacctcaagagcaaacgtgacttattttttctcaacaaggatgcagcactacacatggagcctaagaaagaaaaaatggagccaggcctggaacaaggcaagaaaccaagactagatagcgtcaccagcagcgaaagctttgcgagctccggctttcaggaagataaaagtctgagtgatgttgaggaagaggaggattctgacggtttctacaaggagcccatcactatggaagatctgatttcttacagttttcaagtggccagaggcatggagttcctgtcttccagaaagtgcattcatcgggacctggcagcgagaaacatttttttatctgagaacaacgtggtgaagatttgtgattttggccttgcccgggatatttataagaaccccgattatgtgagaaaaggagatactcgacttcctctgaaatggatggctcctgaatctatctttgacaaaatctacagcaccaagagcgacgtgtggtcttacggagtattgctgtgggaaatcttctccttaggtgggtctccatacccaggagtacaaatggatgaggacttttgcagtcgcctgagggaaggcatgaggatgagagctcctgagtactctactcctgaaatctatcagatcatgctggactgctggcacagagacccaaaagaaaggccaagatttgcagaacttgtggaaaaactaggtgatttgcttcaagcaaatgtacaacaggatggtaaagactacatcccaatcaatgccatactgacaggaaatagtgggtttacatactcaactcctgccttctctgaggacttcttcaaggaaagtatttcagctccgaagtttaattcaggaagctctgatgatgtcagatatgtaaatgctttcaagttcatgagcctggaaagaatcaaaacctttgaagaacttttaccgaatgccacctccatgtttgatgactaccagggcgacagcagcactctgttggcctctcccatgctgaagcgcttcacctggactgacagcaaacccaaggcctcgctcaagattgacttgagagtaaccagtaaaagtaaggagtcggggctgtctgatgtcagcaggcccagtttctgccattccagctgtgggcacgtcagcgaaggcaagcgcaggttcacctacgaccacgctgagctggaaaggaaaatcgcgtgctgctccccgcccccagactacaactcggtggtcctgtactccaccccacccatctagagtttgacacgaagccttatttc

SEQ ID NO: 23 - DNA, 914 bp, Homo sapiens
ctctactgttagtattgcaaagtatgtatgggaaacacagagaattggagctgcgttgaatgcaaacttgaggtgtttcccttgaggaattcttgtcttcaaacgtctgcagagtaatggaccatgttacaactttcctgttcatctgtgaaccatgaaaatggatggcactgatgcattagaccctcagcagcctgcaattgcaaatctgcgaggtttcattcggcccataaagcaaacatttgaacttacacagaatgagcacttaaatacgggtgcaataaatgaagggaaaaacctcagccgtttctccattctgaagatatagcaagcaccgggaaatctaagatttttcatcaacaatatcttctgccagcccagtttggggggaaaaaccccttttacatttttcttcagtaaagtactggaacttacttttccctgtctgtgctaatgagctgattttcagctgatagaaaacaaaatgatagagtacttttttccttggccaagtattttctcatttgtatttaatttcatacattagacagccagtgaaattagacctcaaactaggtcctgatggataatgaatgttatgtcacctttaacagtgaagtggttattataggtcactttctaatttcatattttcccttttgctttctgctgccctcagggtatatagtgtatctctaacctgatttttcaaggttatttttggagcagtttcttaaaacaggcattccctaacttgctcatttaattaatgaaaaattgaactgatgccatggatataaaaacaaatgcaatgtttgattgtcagtgtttctgatttggcaaaaaggaatcatctctatttttttgcaaacaatatcaaagtgcatattttctctcaaaaaaaaaaaaaaaaa

SEQ ID NO: 24 - DNA, 3823 bp, Homo sapiens
aaagcggggcgcaccgcgggcgccggcaacgagccggtgaacgaggcgaggcccgtgcgcccgcggctgcaagcgcccgcctggcggggagaggggccgacggcgtcagcccgggcggcggcatccctaggcgcctggggcgccttcctccggacctggccgctcgctgccccgccctctgcaccccacttctccgaccctccttcccagtcctgcctccccctgccctggcctctgagagccgactgagcccagccccgtgcagcagcggttgcctgtgtcgccgcctagtctccggtcttggtgctctcccgggggtgccccaaggagccagtgcgcgctgcgggctgggaaggaggcgccgctcagctagtcctcctcctcctcctcgtctttctcctcctcctgctgctgctgccgccgccgccgccgtgggtgccgggtccgcgcgcaccccaacacccccaccagctgggcctcggggcacatctccagacttcaatttggctgaaataattcatgccacggacctgtgcacatgcctggaattgagagacacagttaaaagactccaagttgctttctgccttttgaaaactcctgaaaaccatccctttggactctggaattctacacagctcaaccaagactttgcttgaatgtttacattttctgctcgctgtcctacatatcacaatatagtgttcacgttttgttaaaactttggggtgtcaggagttgagcttgctcagcaagccagcatggctaggatgagctttgttatagcagcttgccaattggtgctgggcctactaatgacttcattaaccgagtcttccatacagaatagtgagtgtccacaactttgcgtatgtgaaattcgtccctggtttaccccacagtcaacttacagagaagccaccactgttgattgcaatgacctccgcttaacaaggattcccagtaacctctctagtgacacacaagtgcttctcttacagagcaataacatcgcaaagactgtggatgagctgcagcagcttttcaacttgactgaactagatttctcccaaaacaactttactaacattaaggaggtcgggctggcaaacctaacccagctcacaacgctgcatttggaggaaaatcagattaccgagatgactgattactgtctacaagacctcagcaaccttcaagaactctacatcaaccacaaccaaattagcactatttctgctcatgcttttgcaggcttaaaaaatctattaaggctccacctgaactccaacaaattgaaagttattgatagtcgctggtttgattctacacccaacctggaaattctcatgatcggagaaaaccctgtgattggaattctggatatgaacttcaaacccctcgcaaatttgagaagcttagttttggcaggaatgtatctcactgatattcctggaaatgctttggtgggtctggatagccttgagagcctgtctttttatgataacaaactggttaaagtccctcaacttgccctgcaaaaagttccaaatttgaaattcttagacctcaacaaaaaccccattcacaaaatccaagaaggggacttcaaaaatatgcttcggttaaaagaactgggaatcaacaatatgggcgagctcgtttctgtcgaccgctatgccctggataacttgcctgaactcacaaagctggaagccaccaataaccctaaactctcttacatccaccgcttggctttccgaagtgtccctgctctggaaagcttgatgctgaacaacaatgccttgaatgccatttaccaaaagacagtcgaatccctccccaatctgcgtgagatcagtatccatagcaatcccctcaggtgtgactgtgtgatccactggattaactccaacaaaaccaacatccgcttcatggagcccctgtccatgttctgtgccatgccgcccgaatataaagggcaccaggtgaaggaagttttaatccaggattcgagtgaacagtgcctcccaatgatatctcacgacagcttcccaaatcgtttaaacgtggatatcggcacgacggttttcctagactgtcgagccatggctgagccagaacctgaaatttactgggtcactcccattggaaataagataactgtggaaaccctttcagataaatacaagctaagtagcgaaggtaccttggaaatatctaacatacaaattgaagactcaggaagatacacatgtgttgcccagaatgtccaaggggcagacactcgggtggcaacaattaaggttaatgggacccttctggatggtacccaggtgctaaaaatatacgtcaagcagacagaatcccattccatcttagtgtcctggaaagttaattccaatgtcatgacgtcaaacttaaaatggtcgtctgccaccatgaagattgataaccctcacataacatatactgccagggtcccagtcgatgtccatgaatacaacctaacgcatctgcagccttccacagattatgaagtgtgtctcacagtgtccaatattcatcagcagactcaaaagtcatgcgtaaatgtcacaaccaaaaatgccgccttcgcagtggacatctctgatcaagaaaccagtacagcccttgctgcagtaatggggtctatgtttgccgtcattagccttgcgtccattgctgtgtactttgccaaaagatttaagagaaaaaactaccaccactcattaaaaaagtatatgcaaaaaacctcttcaatcccactaaatgagctgtacccaccactcattaacctctgggaaggtgacagcgagaaagacaaagatggttctgcagacaccaagccaacccaggtcgacacatccagaagctattacatgtggtaactcagaggatattttgcttctggtagtaaggagcacaaagacgtttttgctttattctgcaaaagtgaacaagttgaagacttttgtatttttgactttgctagtttgtggcagagtggagaggacgggtggatatttcaaatttttttagtatagcgtatcgcaagggtttgacacggctgccagcgactctaggcttccagtctgtgtttggtttttattcttatcattattatgattgttattatattattattttattttagttgttgtgctaaactcaataatgctgttctaactacagtgctcaataaaatgattaatgacaggatggggttcccctgtgcttttaccagtagcatgaccccttctgaagccatccgtagaaagtactttgtcctccaaaaagctaacatacggttttgaagcagcattgaaacttttgtagcaatctggtctatagacttttaactcaagaagctaaggctagacttgttaccttcgttgaatgatgttagttgactgtactgtaatgttgtatcaactgaattgaatgtttgcctttaaacaatgaattttctttttctttccttttttttttttttgttgtaatagttaaagaggcttagaacaagctaacaggcaatagaaatatgtatatcagattttttaatgtaacaaactacatgttaattgttatcttattctttttatctttagtagacacttttaaaagaaaagacaagtttgttgtgtttaactcaccaacacgtggtgtataatgaagacagaactataataaattagttttgttctgattttttagaacacttgcaataatgtatcatttatagttcttgctagttgcagtggtaatatttttcacatccataaaaacaactaccaaaata

SEQ ID NO: 25 - DNA, 993 bp, Homo sapiens
tcttctccatttcaaagacactgcatatccttagaaacccttcattacagagggttagtgttaaaaacctcagtggtagggtatatattatataagttaaaaaataaaaaagaagaagccctctaaggctcaagcagaaatattctacagaggggaattttttaggcagaaagaaacagctaggtaagcaacccataactttctcccccattgctttttctcacttcaggaggcccagtatggtggtgagaagaaccagaaggcactcaaaatttaggccagaaggatacgggtatgaggagaaagagataatccttcccacctgcatgcttcctcttaggactttaggtctgcctacatgttgaagaagtttgagttttaggctttgggagcatttccttaaaaaccattgtcatgggggaggaaaaaaaaagggtagtgttaattctgtcttcacacactcagagatcatttggttgggctacattttatgagctatattccataaagatgctgcaaagttcaggtatctggtttaggaaagaaaaaaaaatcccttctttaacccaagaaatgtaatgcaaacaacttgggcctgtgtcacttagtcttctaagaagacttcaaggctgttataaagacccagtggtgggagaaacctgatccggtatagcggggccagaagaagctgcgggaccatgccgcggggacaaggcaaggaaatttgaacaataacagctgctttcagaaataaaccgcgactttgtgcagcgtgtattacctgacgagctgaaatgcattgttaatgagactcgcccgatcattactgctgactgctgtgtgtgttccttttaaaaggccagtcaaagagtcccatccatcatcctcgtaatgcacaatgtaatagccattcatgcccacattaaatttgatccattccacctcttctgggaggatgagcacatctagagtaaataaaataaatcaaagttc

SEQ ID NO: 26 - DNA, 5584 bp, Homo sapiens
acggatccgcgttcagaaaggcgtgcacttcctacgcctgatcccccgcatcgcaacctcgcagcttccccggcgtgcagcgctcatttaccaattcccttcctgggagttgcggcttccctcgctcggccccactcccgtttaccctttccccagctcccgccttagccaggggcttccccgcctgccgctagggctcgggccgaagcgccgctcagcgccagcctgccgctccccgggctccactttcactttcggtcctgggggagctaggccggcggcagtggtggtggcggcggcgcaagggtgagggcggccccagaaccccaggtaggtagagcaagaagatggtgtttctgcccctcaaatggtcccttgcaaccatgtcatttctactttcctcactgttggctctcttaactgtgtccactccttcatggtgtcagagcactgaagcatctccaaaacgtagtgatgggacaccatttccttggaataaaatacgacttcctgagtacgtcatcccagttcattatgatctcttgatccatgcaaaccttaccacgctgaccttctggggaaccacgaaagtagaaatcacagccagtcagcccaccagcaccatcatcctgcatagtcaccacctgcagatatctagggccaccctcaggaagggagctggagagaggctatcggaagaacccctgcaggtcctggaacacccccgtcaggagcaaattgcactgctggctcccgagcccctccttgtcgggctcccgtacacagttgtcattcactatgctggcaatctttcggagactttccacggattttacaaaagcacctacagaaccaaggaaggggaactgaggatactagcatcaacacaatttgaacccactgcagctagaatggcctttccctgctttgatgaacctgccttcaaagcaagtttctcaatcaaaattagaagagagccaaggcacctagccatctccaatatgccattggtgaaatctgtgactgttgctgaaggactcatagaagaccattttgatgtcactgtgaagatgagcacctatctggtggccttcatcatttcagattttgagtctgtcagcaagataaccaagagtggagtcaaggtttctgtttatgctgtgccagacaagataaatcaagcagattatgcactggatgctgcggtgactcttctagaattttatgaggattatttcagcataccgtatcccctacccaaacaagatcttgctgctattcccgactttcagtctggtgctatggaaaactggggactgacaacatatagagaatctgctctgttgtttgatgcagaaaagtcttctgcatcaagtaagcttggcatcacaatgactgtggcccatgaactggctcaccagtggtttgggaacctggtcactatggaatggtggaatgatctttggctaaatgaaggatttgccaaatttatggagtttgtgtctgtcagtgtgacccatcctgaactgaaagttggagattatttctttggcaaatgttttgacgcaatggaggtagatgctttaaattcctcacaccctgtgtctacacctgtggaaaatcctgctcagatccgggagatgtttgatgatgtttcttatgataagggagcttgtattctgaatatgctaagggagtatcttagtgctgacgcatttaaaagtggtattgtacagtatctccagaagcatagctataaaaatacaaaaaacgaggacctgtgggatagtatggcaagtatttgccctacagatggtgtaaaagggatggatggcttttgctctagaagtcaacattcatcttcatcctcacattggcatcaggaaggggtggatgtgaaaaccatgatgaacacttggacactgcagaagggttttcccctaataaccatcacagtgagggggaggaatgtacacatgaagcaagagcactacatgaagggctctgacggcgccccggacactgggtacctgtggcatgttccattgacattcatcaccagcaaatccgacatggtccatcgatttttgctaaaaacaaaaacagatgtgctcatcctcccagaagaggtggaatggatcaaatttaatgtgggcatgaatggctattacattgtgcattacgaggatgatggatgggactctttgactggccttttaaaaggaacacacacagcagtcagcagtaatgatcgggcgagtctcattaacaatgcatttcagctcgtcagcattgggaagctgtccattgaaaaggccttggatttatccctgtacttgaaacatgaaactgaaattatgcccgtgtttcaaggtttgaatgagctgattcctatgtataagttaatggagaaaagagatatgaatgaagtggaaactcaattcaaggccttcctcatcaggctgctaagggacctcattgataagcagacatggacagacgagggctcagtctcagagcgaatgctgcggagtcaactactactcctcgcctgtgtgcacaactatcagccgtgcgtacagagggcagaaggctatttcagaaagtggaaggaatccaatggaaacttgagcctgcctgtcgacgtgaccttggcagtgtttgctgtgggggcccagagcacagaaggctgggattttctttatagtaaatatcagttttctttgtccagtactgagaaaagccaaattgaatttgccctctgcagaacccaaaataaggaaaagcttcaatggctactagatgaaagctttaagggagataaaataaaaactcaggagtttccacaaattcttacactcattggcaggaacccagtaggatacccactggcctggcaatttctgaggaaaaactggaacaaacttgtacaaaagtttgaacttggctcatcttccatagcccacatggtaatgggtacaacaaatcaattctccacaagaacacggcttgaagaggtaaaaggattcttcagctctttgaaagaaaatggttctcagctccgttgtgtccaacagacaattgaaaccattgaagaaaacatcggttggatggataagaattttgataaaatcagagtgtggctgcaaagtgaaaagcttgaacatgatcctgaagctgacgcaacaggatgaaaatccatcagaatctcagactacagcactaaatatgctttgatgctacatcaaacggaatggaagcatagctgacttcgctaaagttacttcatctccatctagcaaatgaggcactgttctcaaccaaaggagatggggatctggtttagggcaatccctttataatttgatgtgctgtggtctccttggtaatgtataatttggtattgcacaggtgattagtcaaggaagtctggaaaagctttggtcccacagccttgcctcacagcatgtaaataattaaaacaatattgatgctgaggttcttctactgctagtatgaaagtgacaaatttttactggtgtgaattgggaagaaaacaatgctattccatgacgtttgtaaaatgtttgtaaaagctcaaacatgacgattccataaaataaacttgaggttaaataatgggtagtaaattatagaatgtataagaaaaaatataaaggagaaaatcaattatcaggaaagctaaagaacttttcaaatctagtaatttgaatatagacacaatgcactttattgcactttcaattcttataaagcaacaataatattaaggtccttgactatgtgtacaatgttttcacatatatagtttcatttaatcatttcaaagttaatctctgccatctcgctaaatcatcagtctcggctcttctgaaatagaaggtgcctgatcttcctaataattctgcctattttcatttgctttaaacaggcgccctattttctttctagttgtggctgcgcaaaaacatttatctcccaaataagatgtgctgcttaccgaggtatcacggggtggggctccagcttgggtcgttgaagctggggtttgggaaaccacttcagagatggcagcagcaagtttagcatcttcaaatttcttttattgaaaaaaattttattagtaacatgttgtatataaaattatgagcacaatgccatcacttaactataactcttaaagatagcttaatgactgtttattctcttgaccaaatagactcataataacatataattttaaaagaaatttaaattctttcttctctattgtattattttatacaatttgctatttctatttccttctcatattgattattctaaatactatgcaataatataacttagagttccacggtttgtttacacatttcctgttgtacatttaggttattcaaagttttcagctcttttaaaattgctctgaataagttctagtgagtgagttatggtgctggctatattttgctaaactgccctctcaaatgttgctaggaattcatactgcgaaaagcaatgaataagcatgcctgttttcccatggccttgcttgccagaatttgacttttattatgataatcagtgtaaaatgatatactactattgcttgtatattgtggtatacggtgtcaggtttcagggttttttttcaacgttaaatattctagaaactttctgaaataatttctgtttaaaaatattgaatatttgcttcatttcaaatactcccttttgacaaaaaaacttaggtataactgttgatgaaaaaccagaaaaaagtccagaactctttggtgactccaactatggatagcttattttgaaaaaggagaattgcaaattttaccaaaagatggagaaaagcacattaaaaagataccaacattcagaaattcatttcagcatgttattattggaaattatttaaactaatttagataactataagatacttattgtccatttataccctgtaaagccgttttagaatgtaatattttaggtaatccaaaatgtactaaattaaattcatttttagttatgagaaatctttgcttatatgacaaatgaaaagaataacaagttgtcaaatgaaaagaatgacattgaaacatttgtattgtctcttcttaaactatcttattgacttattatttaagccttttaatactaagtatgaaacaacctatggtctggaaatttgtatcgcaaagctatatgtgcatatgttatttaattcatctaatgctacacaaaagcataaaataatgatttttcactctctttaaaaatactaaatcatttatgtccatttctcaattttttcattgatctatgctttgagtttgctttctcaacattattgtattttccacttattattactgtataacatatgctagtgtttagttggattaatcttacctaaaagtactgaaaaatgctttttagtactttttcatattttatacatttattttccgaatgtatcattgaataattttattgagttataaaagtatcttattgctatttaataaaaaattaacacataaaatga

SEQ ID NO: 27 - DNA, 2341 bp, Homo sapiens
ggctgcgtggataaaacccaaagggaatccagaatcaagctcggcctcgcaggagtttataacttttgtacttttccctcccttgtaacgaaaagctaatcgcaaacgtttctaggaacacatttttctcctgatagacgttcccaaggtggagggcgcgagaagactggcccgtggggtgaggagccccggcccgacctcagcggcgccacaaagccatgaagaggggaaataattttccaaggggctaagagttggactttctgaagccatcgaaacctgctgctaggctctcccgctggggttttgagagaggtaggtgaggagggatcaaacatctccacccgctcttgaaagaaccgccttgagctgcgcccaacttctgcaatcccggttttccccaagttcaaggggaagggccctggcgcccaatgcccagcctccccgacaacatcctcgccagggtgcgcctgccccgcgggtccgctgtccccggcgggcggccctgggttcgaggacccggcttcccggaggcgcgactctccagcaaacaagcggagctccagaaaaaacaccgacgcgaagggggtggacagagggtgggaagccgcggggttgtcgcccacccggaggcggaatgtaacgcgctgtgggaaacgtggcaggagccctgaggcggcgtctttttcctcaggcgcccgccgagggtcccgcggaaagcctcgcgggccgccctcgcggcccgcagccccgctctggcgccgagccggagcccatgcaacctggttccatcccctgcccctcccctgtccccggtgcgccgcccgccagcgagccttcgacgtggccgcaggggcgacgggaccaccctcccccgatacccacagccccgccatgtctgcctttccccggcccggtctcctcaccgccgctgccgccgccgctcttcggccggagattcggcggcccagaccgtgtcctggccgggaactgagggctccgcctcaacgggcccgcgctgggcaacagggagcgcagcgagcctcgtccggcgcgtgcggctcccgcgtcgtcgggcggccaagcgcgcgttgagaggactggcgggcggacgagcgcgcacacgagtgagcgcagccccaaagcggcgcgcagggggctcgcggcccggaagaggggaggggcgatgacccgggaaagggttggcgcgcgcgggatcggctcgcgcgcctgaggggcggtgccgggggcggggcttcgccgcgaggccacccccgagccccgggcctagccgcacgggaggcgacacaccctcgccctaccctgagcctggcgcagaccctcgaccgcgacggcgcggtccgcagaaccgcgggcttccctccccggcaaaaagcggcgctgaaggcctgtgggacccccgcccggccgccctacgtgcgtcagcgccttcgggggtcgcgcgggcttggtcctttcggctgtctcggcctttttgtttccccctcaggtctctccacgctgcacttgacaccctcagggcggaatggcgagttctagacccagctctctagacccggggcttcatgggggacacggactcgacgggaaggggaacttggcatttatggacagatcctcatccttcttgctgtagtcaattacacgcacgttaaccgtgcagccgccctgctgtattttaggcggttgttggcactcctagttgggcccttccctggcccctcacagcagggcctgcctcctgtggacgcttgtgtgctgccctggcaccggccactgtgttttgcataaaggagaggctccaaagatgttggccaaaggaatgaagccttgagagtccaggctttctatttctgagacactctactcaaggacttcccagatgcaaagcttcatctttgagcaaatacaaatatggagagggaacattaactttctgaagaaaagaaggaacatcttttcaaccttttatattgagttaacaccatggtcttagttttgtgaaagcactttaatacatccaactagcgggaggaatacaagatattcctacctttttattattattattaaaagagggctaaaccatgttgtaaactacttaagaactgaatgttcaagttgtacagtatattggccagattccttactgcccactaggcttgggagcatgttttcagctcagttttatgaatgttttaaatttgtattactgatctttgttatagccaattagcaattcaaaatggtaatttttttagcagtaataaatataccatcccccaaacagtaaaaaaaaaaaaaaaaaaaaaaaaaaaaa

SEQ ID NO: 28 - DNA, 1596 bp, Homo sapiens
ccctcttccgggccgcgagccccctgcgcgccgctttggggctgcgctcactcgtgtgcgcgctcgtccgcccgccagtcctctcaacgcgcgcttggccgcccgacgacgcgggagccgcacgcgccggacgaggctcgctgcgctccctgttgcccagcgcgggcccgttgaggcggagccctcagttcccggccaggacacggtctgggccgccgaatctccggccgaagagcggcggcggcagcggcgggaaaaaaatgaagaatgaaattgctgccgttgtcttctttttcacaaggctagttcgaaaacatgataagttgaaaaaagaggcagttgagaggtttgctgagaaattgaccctaatacttcaagaaaaatataaaaatcactggtatccagaaaaaccatcgaaaggacaggcctacagatgtattcgtgtcaataaatttcagagagttgatcctgatgtcctgaaagcctgtgaaaacagctgcatcttgtatagtgacctgggcttgccaaaggagctcactctctgggtggacccatgtgaggtgtgctgtcgtagagatggggtttcaccatgttggccagactgctctcaaactcctgacctcgtgatccgcccgccttggcctcccaaagcgctggattacaggcgtgagccactgcgcccggcctcctcctttttgattatgtatggagagaaaaacaatgcattcattgttgccagctttgaaaataaagatgagaacaaggatgagatctccaggaaagttaccagggcccttgataaggttacctctgattatcattcaggatcctcttcttcagatgaagaaacaagtaaggaaatggaagtgaaacccagttcggtgactgcagccgcaagtcctgtgtaccagatttcagaacttatatttccacctcttccaatgtggcaccctttgcccagaaaaaagccaggaatgtatcgagggaatggccatcagaatcactatcctcctcctgttccatttggttatccaaatcagggaagaaaaaataaaccatatcgcccaattccagtgacatgggtacctcctcctggaatgcattgtgaccggaatcactggattaatcctcacatgttagcacctcactaacttcgtttttgattgtgttggtgtcatgttgagaaaaaggtagaataaaccttactacacattaaaagttaaaagttcttactaatagtagtgaagttagatgggccaaaccatcaaacttatttttatagaagttattgagaataatctttcttaaaaaatatatgcactttagatattgatatagtttgagaaattttattaaagttagtcaagtgcctaagtttttaatattggacttgagtatttatatattgtgcatcaactctgttggatacgagaacactgtagaagtggacgatttgttctagcacctttgagaatttactttatggagcgtatgtaagttatttatatacaaggaaatctattttatgtcgttgtttaagagaattgtgtgaaatcatgtagttgcaaataaaaaatagtttgaggcatgacaaaa

SEQ ID NO: 29 - DNA, 1810 bp, Homo sapiens
tctcccggaggcgcatgatgtcctcggccaggttgtcgcgctccacctcgacgcgggctttgtcgttggttagctggtccacctgccggcgcagctcccgcatctcctcctcgtagaggtcccccaggcgcgacttgccttggcccttgagctgctcgagctcggccagcaggatcttattctgctgctccaggaagcgcaccttgtcgatgtagttggcgaagcggtcattcagctcctgcagctccaccttctcgttggtgcgggtgttcttgaactcggtgttgatggcgtcggccagcgagaagtccaccgagtcctgcaggagccgcaccccgggcacgctgctccgcaggcgcacggcagaggagcgcgtggcatacacgccgcccggggacgaggcgtagaggctgcggctggtgctggggcgcagcgcgctgcccaggctgtaggtgcgggtggacgtagtcacgtagctccggctggagctcggccggctcgcggtgcccgggccgccgaacatcctgcggtaggaggacgaggacacggacctggtggacatggctgcggagggtggcgatggcctgggcggcggcggtggcgcggactggctcccggagaagaggcgaacgagggcgcgacagcaaagctccctttggatgacatagatttattacttagtagtatattatgtattggctgtcccacattttgaaatttaatggactcgtggtgatcagaataaaagaagccttcgatgtgaggcccaggcattgagtaccatgtgtgcgattcacaagccttggcatctgaacaattttttggaaggatccatcaggacaacacgtcgggggtgtactagtgaagtgattttccaaatgtgctactcagacctgtagcatcagcatcaccggatatacaagacttaccaggtgattctgagaaccactgtgctagtgaattgttcctgtctgtggccacgtagaataagaaaaccatagggttggaaaatggggaaactggtgagtgttcgcttatgaggaaatgaagaatagatagaaaagaattagttaacttttggaattcaaaagagaagcagtttgtaaaagccaggaatttgatttgaaggactaattgcttgcagaatctttgctttctcagagaggggcaatccagatcactaggttaccgtgaaatatgttggtatggctctaaaattctagaataatctctctagtgagaaaaggcatacctttccatattaggacaaaacttcaaatcaggtttgtaaaatcctataagattattgtatcccattgccatggccaacttgtttgtctctggagatcccagtttctacatctgaaaaccatatgcattcctgctcaccaggaattatctcgctgaatgaagcaagagattcaggatgtgtaagagaatttatgaagcatttggattaccaagaaatgtgaagtataaagatcaagaatgattattacaaacactgatggtaaagaggtgtttcgaagtcgatgcaaaaaatgagttgcttatttcagtctctctttgatatatctgcctttttagtgctgactctatcatgtattcactatttgattttcagtgaatcacattttttaaagcttttaatctgtttcctcaaaaaatatattttttaaaaatatttactcaggattgttgtgagaataaaattgattcgattgatatttcaaaaaagaaatatatttttaaaaaataaagcaataccatttttggaaaaaaaaaaaaaaaaaaaaaaaaaaaaaaaaaa

SEQ ID NO: 30 - DNA, 2151 bp, Homo sapiens
gcctctccaaaggctgcagaagtttcttgctaacaaaaagtccgcacattcgagcaaagacaggctttagcgagttattaaaaacttaggggcgctcttgtcccccacagggcccgaccgcacacagcaaggcgatggcccagctgtaagttggtagcactgagaactagcagcgcgcgcggagcccgctgagacttgaatcaatctggtctaacggtttcccctaaaccgctaggagccctcaatcggcgggacagcagggcgcgtcctctgccactctcgctccgaggtccccgcgccagagacgcagccgcgctcccaccacccacacccaccgcgccctcgttcgcctcttctccgggagccagtccgcgccaccgccgccgcccaggccatcgccaccctccgcagccatgtccaccaggtccgtgtcctcgtcctcctaccgcaggatgttcggcggcccgggcaccgcgagccggccgagctccagccggagctacgtgactacgtccacccgcacctacagcctgggcagcgcgctgcgccccagcaccagccgcagcctctacgcctcgtccccgggcggcgtgtatgccacgcgctcctctgccgtgcgcctgcggagcagcgtgcccggggtgcggctcctgcaggactcggtggacttctcgctggccgacgccatcaacaccgagttcaagaacacccgcaccaacgagaaggtggagctgcaggagctgaatgaccgcttcgccaactacatcgacaaggtgcgcttcctggagcagcagaataagatcctgctggccgagctcgagcagctcaagggccaaggcaagtcgcgcctgggggacctctacgaggaggagatgcgggagctgcgccggcaggtggaccagctaaccaacgacaaagcccgcgtcgaggtggagcgcgacaacctggccgaggacatcatgcgcctccgggagaaattgcaggaggagatgcttcagagagaggaagccgaaaacaccctgcaatctttcagacaggatgttgacaatgcgtctctggcacgtcttgaccttgaacgcaaagtggaatctttgcaagaagagattgcctttttgaagaaactccacgaagaggaaatccaggagctgcaggctcagattcaggaacagcatgtccaaatcgatgtggatgtttccaagcctgacctcacggctgccctgcgtgacgtacgtcagcaatatgaaagtgtggctgccaagaacctgcaggaggcagaagaatggtacaaatccaagtttgctgacctctctgaggctgccaaccggaacaatgacgccctgcgccaggcaaagcaggagtccactgagtaccggagacaggtgcagtccctcacctgtgaagtggatgcccttaaaggaaccaatgagtccctggaacgccagatgcgtgaaatggaagagaactttgccgttgaagctgctaactaccaagacactattggccgcctgcaggatgagattcagaatatgaaggaggaaatggctcgtcaccttcgtgaataccaagacctgctcaatgttaagatggcccttgacattgagattgccacctacaggaagctgctggaaggcgaggagagcaggatttctctgcctcttccaaacttttcctccctgaacctgagggaaactaatctggattcactccctctggttgatacccactcaaaaaggacacttctgattaagacggttgaaactagagatggacaggttatcaacgaaacttctcagcatcacgatgaccttgaataaaaattgcacacactcagtgcagcaatatattaccagcaagaataaaaaagaaatccatatcttaaagaaacagctttcaagtgcctttctgcagtttttcaggagcgcaagatagatttggaataggaataagctctagttcttaacaaccgacactcctacaagatttagaaaaaagtttacaacataatctagtttacagaaaaatcttgtgctagaatactttttaaaaggtattttgaataccattaaaactgcttttttttttccagcaagtatccaaccaacttggttctgcttcaataaatctttggaaaaactcaaaaaaaaaaaaaaa

SEQ ID NO: 31 - DNA, 590 bp, Homo sapiens
caaaatagtcaggaaagttatgaagtaacatcatataatactttatccccaacaatattaaatgtgattacaattttacagaatattcaaatcaaaatgttatataagacaaattattattcttgcttttacatctccttagaaggagaaacattcaattgttttaaaaatgttctatttatttttcagaaatgaggtgttgccttcttgcccaggctagtatgcagtggcacaatcatagctcactgcagccttgaattcttgggctcaagtgatcttcctgccccaagcctcccaagtagctgggaccacaggcacgtgccaccatgcctggctgatttttttttttttttaatgttttgtagagacaggatctcagtatgttgcgcaggctggtctttaattcctgggctcaagtgatcctcccatagatttgtctgtgttttctctaaataggttataaataccttgagggaggaatcacaaataactaaaagaaacttttgtagagactatcagagtattatttccttgtggacacagtaaatgtatgatgatgagaaatcataataaactacaacattttctcc

SEQ ID NO: 32 - DNA, 3050 bp, Homo sapiens
tttttttgcttctgccccagatctttcctggacagtgcgtctcagcagttcagatccgggggcccccagctgacagagggcgtggggggttaaggcattaacccctcccagcctcttcctgaagaaaccacccagccttggcgcggcgctgggtgacttcgcgtagcaggcagggaactggccgcggcgagcgggactggccattggagtgctccgctgcggagggaggggaccccgactcgagtaagtttgcgagagcactacgcagtcagtcgggggcagcagcaagatgcgaagcgagccgtacagatcccgggctctccgaacgcaacttcgccctgcttgagcgaggctgcggtttccgaggccctctccagccaaggaaaagctacacaaaaagcctggatcactcatcgaaccacccctgaagccagtgaaggctctctcgcctcgccctctagcgttcgtctggagtagcgccaccccggcttcctggggacacagggttggcaccatggggcccaccagcgtcccgctggtcaaggcccaccgcagctcggtctctgactacgtcaactatgatatcatcgtccggcattacaactacacgggaaagctgaatatcagcgcggacaaggagaacagcattaaactgacctcggtggtgttcattctcatctgctgctttatcatcctggagaacatctttgtcttgctgaccatttggaaaaccaagaaattccaccgacccatgtactattttattggcaatctggccctctcagacctgttggcaggagtagcctacacagctaacctgctcttgtctggggccaccacctacaagctcactcccgcccagtggtttctgcgggaagggagtatgtttgtggccctgtcagcctccgtgttcagtctcctcgccatcgccattgagcgctatatcacaatgctgaaaatgaaactccacaacgggagcaataacttccgcctcttcctgctaatcagcgcctgctgggtcatctccctcatcctgggtggcctgcctatcatgggctggaactgcatcagtgcgctgtccagctgctccaccgtgctgccgctctaccacaagcactatatcctcttctgcaccacggtcttcactctgcttctgctctccatcgtcattctgtactgcagaatctactccttggtcaggactcggagccgccgcctgacgttccgcaagaacatttccaaggccagccgcagctctgagaagtcgctggcgctgctcaagaccgtaattatcgtcctgagcgtcttcatcgcctgctgggcaccgctcttcatcctgctcctgctggatgtgggctgcaaggtgaagacctgtgacatcctcttcagagcggagtacttcctggtgttagctgtgctcaactccggcaccaaccccatcatttacactctgaccaacaaggagatgcgtcgggccttcatccggatcatgtcctgctgcaagtgcccgagcggagactctgctggcaaattcaagcgacccatcatcgccggcatggaattcagccgcagcaaatcggacaattcctcccacccccagaaagacgaaggggacaacccagagaccattatgtcttctggaaacgtcaactcttcttcctagaactggaagctgtccacccaccggaagcgctctttacttggtcgctggccaccccagtgtttggaaaaaaatctctgggcttcgactgctgccagggaggagctgctgcaagccagagggaggaagggggagaatacgaacagcctggtggtgtcgggtgttggtgggtagagttagttcctgtgaacaatgcactgggaagggtggagatcaggtcccggcctggaatatattttctacccccctggagctttgattttgcactgagccaaaggtctagcattgtcaagctcctaaagggttcatttggcccctcctcaaagactaatgtccccatgtgaaagcgtctctttgtctggagctttgaggagatgttttccttcactttagtttcaaacccaagtgagtgtgtgcacttctgcttctttagggatgccctgtacatcccacaccccaccctcccttcccttcatacccctcctcaacgttcttttactttatactttaactacctgagagttatcagagctggggttgtggaatgatcgatcatctatagcaaataggctatgttgagtacgtaggctgtgggaagatgaagatggtttggaggtgtaaaacaatgtccttcgctgaggccaaagtttccatgtaagcgggatccgttttttggaatttggttgaagtcactttgatttctttaaaaaacatcttttcaatgaaatgtgttaccatttcatatccattgaagccgaaatctgcataaggaagcccactttatctaaatgatattagccaggatccttggtgtcctaggagaaacagacaagcaaaacaaagtgaaaaccgaatggattaacttttgcaaaccaagggagatttcttagcaaatgagtctaacaaatatgacatctgtctttggcacttttgttgatgtttatttcagaatgttgtgtgattcatttcaagcaacaacatggttgtattttgttgtgttaaaagtacttttcttgatttttgaatgtatttgtttcagcagaagtcattttattggatttttctaacccgtgttaacaccattgaatgtgtatttcttaagaaaataccaccctcttgtgcccttaaaagcattactttaactggtagggaacgccagaaacttttcagtccagctattcattagatagtaattgaagatatgtataaatattacaaagaataaaaatatattactgtctctttagtatggttttcagtgcaattaaaccgagagatgtcttgtttttttaaaaagaatagtatttaataggtttctgacttttgtggatcattttgcacatagctttatcaacttttaaacattaataaactgatttttttaaagatc

SEQ ID NO: 33 - DNA, 3186 bp, Homo sapiens
ctagcatggcagcttccccctcctcgatgaagcccagcacctgggtctggaagccctgcagggcggccgcagcatctgcaaacagccggctcaccctctcccgctctgctacggctgcactctgcacaggacgacagtagagggggcaatgagggcaaagaaccgtcccggactgtacccccctccttccctgaccccggcttcccaccggccctcttgaggggacagaggggccagactgagcctgtcctggatctgggccaggtcaggggacacttgtgctcagtaggtatgggcatggtttgtttggagtggattgttccatgctgagggaggggtgtttgaccttgatgagggccactgtgcgcctggactgtgcaatgccagcacccagctcgtccatgcggtcctccacggcgctcaggactttggactgctcagcctgtggacaacaccctccatgagtgtgaggtctggcagaggccacagccctacgctcgggtcccaggccttgccaggggagagaagggggtgactggggagtggtatctgtgcatcaggtgggactggagtggccacgatgtcccataatatgccagaggtgcctgcagcccaggctggtgcctttcctgaacgcctggggggcgtgcaggctgactgggtgtctgcctcgggtctctgacaggaggcctagtacagggccgtccgctggtctcagtgttgctcagatgaatttgagaacttcctccggcggggaagaggcagagagtaagccagttaggttgggaggtgggcggcggaggggtgactgcacaaggaggcattaagctgtcctgaggatcactgttcccaaagagcctgcatccacagtccaggcctggaaaaaaggggattccgggcaaatcccctaggtcttatgagtcactgctccaggcaggagaaacggaaaaccccttggccagaggcgccaagaccgggctggaggccaaaggaaactgggggcgggaggtagaggccgcggctcacatgctcccaccactggcccccggaacagctggacagagaatctgtgtgcccggtcgccctctgccgccctcctgccgcctgccacacccatcgaaagctgctgcctcagcccagggcaaatctgagctgagaccggcgtgaccctccgggcgggagcagcagccacgctaccggaaccccgcgggcctaacccggctccctcccgcctacacccccatagaccccggcccggcttcaggtcccctgcccgctcttcgcgtccttgtcctgttctcacgtccctccttcctcccttcccatctccatcaccgccagcagatgaccttgtcaaggctggaagggaccgcaaagatcctcaggttagagggggagattaagacccaacccgggaagactggggggtccaaggtcctcggcccgctcgcgcgcggtcggtcaccagccccggctcacccgagtgtgcccccggagcccacctcctgaagcgcgcgctcctgctccagcggcaccagctcgtggccgcggtgctcctgtgcggcgcaggcctcgcacagacacacgcgctccgcgcggcagtagcgctcgagcggccgtaggtggcgcgggcacaggctctcctctagccggcgcagcggcggcaccaggcggtgtccgcggagggcggggctgcgctcgtgcgggcccaggtgcgcggggcaaaaggaggcgaggcaggagaggcaggacagcgcggcgggcagggccgcgccctcggggcacgcgtcgcagcgcactggctcttcgcccgcgggccacggctcgggagcgcagggggccgacggctccgggacactgggcagcgcgctgggtgccgagggctccggggccagggcaggggccgggccggggccggacccggggcccgagccctggcggagctgcagcagctcggacagcgtgtggttcttgcggagctgaaggccgtcggggaagggctcctggcacagcgggcagcgggccgcgcctccgggtccgccggctccactcgcgccacgatgcggccagagcgcgcccaggcaggcgagacagaagttgtggccgcagggcagcgtcaccggctcccggagtggctctaggcagatggggcagctgaagggtccactgccgtccatgactccgcggccgcccagggcgccgcggattgtgctccggcctgggagggacccgggccgttcgcgccgcggcacctccccctgggacctaggccagggctccggcccccgcccgcccccagctcggcccgccccgcagcaccgcccgcctgcgggcccgcggactcccagtccccgcccagacaacgcagggaggcctccgagcccgcgcgacccccaggggagtccgcgtggtcctaaggaagggcgctgctgaaagggtgtgaaactgagtgagcgcgggcggagaacgcgagaggtacagtggaaggaatgggaaggactcagcctccatccctctggtccttctgggccaggccagcgctccgcaggggctgggcgcgtctagctgtgtcatggctgcacctttgcgctcgacccctccctggcacccttagcaaagttcctgagctgctgactgcagagaaaaataactgctgatgtacatatgtttttgccccttagcgactctttttttttttttttttttttgagacagagttcctctcttgtcgcccaggctggagtgcaggggcgctatctcggctcactgcaacctccgcctcccggattcaagtgattctcctgccttagcctcccaagtagctggaattataggcgcttgccaccacaccaggcaaatttttgtatttttttgtagagacggagtttcaccttgtccaggctggtcttgaactcgtgacctcaggtgatccacctgcctcggcctcccaaagtgctgggattacaggcgtgagccacagcgcccagctcctttagcgactctttaccccaagtagggaacttggacttttctagacctgaaatgggaagttgacgtcattccctaagtcccttgtctgaggaatgtagttctggcccctggacactggggacacctgtggtggggttgggggttccccaactggaagtatttaaagctcatctaattatgcaaaaaaaaaaaaag

SEQ ID NO: 34 - DNA, 2268 bp, Homo sapiens
ggcggcgccctgggcggccgcggagtcatggacggcagtggacccttcagctgccccatctgcctagagccactccgggagccggtgacgctgccctgcggccacaacttctgtctcgcctgcctgggcgcgctctggccgcatcgtggcgcgagtggagccggcggacccggaggcgcggcccgctgcccgctgtgccaggagcccttccccgacggccttcagctccgcaagaaccacacgctgtccgagctgctgcagctccgccagggctcgggccccgggtccggccccggcccggcccctgccctggccccggagccctcggcacccagcgcgctgcccagtgtcccggagccgtcggccccctgcgctcccgagccgtggcccgcgggcgaagagccagtgcgctgcgacgcgtgccccgagggcgcggccctgcccgccgcgctgtcctgcctctcctgcctcgcctccttttgccccgcgcacctgggcccgcacgagcgcagccccgccctccgcggacaccgcctggtgccgccgctgcgccggctagaggagagcctgtgcccgcgccacctacggccgctcgagcgctactgccgcgcggagcgcgtgtgtctgtgcgaggcctgcgccgcacaggagcaccgcggccacgagctggtgccgctggagcaggagcgcgcgcttcaggaggctgagcagtccaaagtcctgagcgccgtggaggaccgcatggacgagctgggtgctggcattgcacagtccaggcgcacagtggccctcatcaagagtgcagccgtagcagagcgggagagggtgagccggctgtttgcagatgctgcggccgccctgcagggcttccagacccaggtgctgggcttcatcgaggagggggaagctgccatgctaggccgctcccagggtgacctgcggcgacaggaggaacagcgcagccgcctgagccgagcccgccagaatctcagccaggtccctgaagctgactcagtcagcttcctgcaggagctgctggcactaaggctggccctggaggatgggtgtggccctgggcctggacccccgagggagctcagcttcaccaaatcatcccaagctgtccgtgcagtgagagacatgctggccgtggcctgcgtcaaccagtgggagcagctgagggggccgggtggcaacgaggatgggccacagaagctggactcggaagctgatgctgagccccaagacctcgagagtacgaacctcttggagagtgaagctcccagggactatttcctcaagtttgcctatattgtggatttggacagcgacacagcagacaagttcctgcagctgtttggaaccaaaggtgtcaagagggtgctgtgtcctatcaactaccccttgtcgcccacccgcttcacccattgtgagcaggtgctgggcgagggtgccctggaccgaggcacctactactgggaggtggagattatcgagggctgggtcagcatgggggtcatggccgaagacttctccccacaagagccctacgaccgcggccggctgggccgcaacgcccactcctgctgcctgcagtggaatggacgcagcttctccgtctggtttcatgggctggaggctcccctgccccaccccttctcgcccacggttggggtctgcctggaatacgctgaccgtgccttggccttctatgctgtacgggacggcaagatgagcctcctgcggaggctgaaggcctcccggccccgccggggtggcatcccggcctcccccattgaccccttccagagccgcctggacagtcactttgcggggctcttcacccacagactcaagcctgccttcttcctggagagtgtggacgcccacttgcagatcgggcccctcaagaagtcctgcatatccgtgctgaagaggaggtgatgccgggcacgggcgctcctgctgccgtctctgctccaggaagctgcctcctctgggccctctccttcgtctgggaaggcaccagcatgagtcccacacacccagccttctcatttctagaggcttccacctttttatacactcagccttccctctcccaggcaggaggacccccagaccctgttcccctgcagacctcacttctgggagacagagctacagctgggacagctccaagctaccctaacccctcctttcccaggtttctagaatagtgtctggcatgtagtagatgctcaataaacacttgttgaatgaaaaaaa

SEQ ID NO: 35 - DNA, 2113 bp, Homo sapiens
gtattttttgctaataaaatcaagtatcaaattacttttggtgagggatgggcaactgtattgtactgctgataatttgcagtcaggaaaaattttgttttccttctcataacaacactgttaagccatgattcagtttactttcacatttttcttgagccaagcatagaatgctattaaattaaattttattggttttaaattataattttagcctgtttcaatctttgtgaaagctaattctatcactcaacataattaagttattctcctcttgggctcgtgccacccaaaatgatgagctagctttcgacaactgagtcttggtaatttacaaaactgttcagaaagacgaaccaaaatttgaatggcttgctgtgttctgtataaatttataatcgaaagtctgatcatggctacaggtttttttttttttaacttgttctgtttttcaacaaacaatatacttatttgcaattaaatgaaaataaagcattgaattctgaagttttagagaatgaccttagagatttttagtggttgtcagccttagcctcctatcaaaatcatctgggaactattaaaagcatttaatgttgtgaccctacctagacctattaaattccattctctggggtgaagccaggccaccagttattttaaaagttctccaagtgcttctaatgtgcagcacaggttgggagaatctgatctagcttagcacttaaaggttatagtgctaccactacataacacttctatattgagtttactgtagtatattaatggagctatcttaagtggaaaatgtctgatcaatcattaaatagcaatctaagtgaaatctataactaaaatttctatctggaaagacaaaaagaaaaaccaacttctctttgagggtatgctatacccagagacttggctagtgctaaggagggcatggtagacttcccgaggtcacacaaagaacaagcagctcatgcttggttctacccaatagtctgagtcccctcagtcactctgttttagcctctaaaccactaggacgtgatttgagaaatttcatgaagcctcaaggtgttctttcaggttatttgtacaatggggaagaaaatatctatagttcaacctctttcccatactcatgacagatcacctggcctgtgcttcagtattttacagattgttcatctgtggctgaaaagctcttatcaaaatgctcttacttttattgagctgataaagttgccatcctaacttctatccattggttctggctctgcctcctggaggaacacagactatgttgattgtcttttccctgtgttagcaatgcagatggattgtaattttcctaaatctgctcttcaagaaaagatttcaagttttgaaaaccatatcatcatcctgtcctcggtgacacctgtttctctcctttgtcaacatcacctttcacaatgtgtcacctggatagcagatcgaacaacctggctataatagtggttctgatagcctcctttgatctcgtcattgtattctagaaatgaagcccaagattttatgtatttttaccgcggtcataccacgttgatttaaaagatcaatgattttcgagccaggctggacattaaaatcacttgggagagctttgaaaaaatatagcgatatcctgttttacccttgatcaattaaatcagaatctctggggataaggcccaggtacagtgatttttaaaagtacttcaatgaccctaatatgcatctaagtgagttctatccttaaatgtggtttctctcaatttgtaggtaaatacaatattttgttgttgtttgaatgtaaatatcaggcttttgatctgtcctagtttaattttctgataatagtagtcccagaattttcaaatgcaattctttaagattgcaatgaagttagaactcttacgttactgtttatgtccacaaaatgtttagaaaagtctgaaaaggattttgaaaaagattaacagtgttaatatttcttatattgttgggtttatgcataatataatttggttaatatgtatcataaataaaagtattttaagatcacaaaaagaaaaaaaaaaaaaac

SEQ ID NO: 36 - DNA, 4072 bp, Homo sapiens
acaccgccctcccgccagactcccggcggctcctcctccctctcccaaacccactcccaaagctaagtgcaggcttccccgttccagccagaagcgctgcgtgagcctccacacgtagccgcaggcagctccttaaatagcgtccgcgctgagcaaacagtccagacgtggggcccaggagggcgagctgaggcgaccgcaccgggcgcgcagcggcggcgggtcagccggcggccaatagccagggcgcggcccgccccgtcgcctcccctcggggagcctataaggcctccgcagcgccccgggcgcctgctgctccgtgcctccaccgacgacctcactcagctgcgttacgcgccgctccggctgccggccgcgcgccttgcccgccggctcccgcccgcaatcggcggctcagggcggacccgggtctctgcgttctcgcgagaagcgcggcgctgcggggccgtgggcgcctgagcccgcgcggccctcgagggccgaatatggggggatgcacggtgaagcctcagctgctgctcctggcgctcgtcctccacccctggaatccctgtctgggtgcggactcggagaagccctcgagcatccccacagataaattattagtcataactgtagcaacaaaagaaagtgatggattccatcgatttatgcagtcagccaaatatttcaattatactgtgaaggtccttggtcaaggagaagaatggagaggtggtgatggaattaatagtattggagggggccagaaagtgagattaatgaaagaagtcatggaacactatgctgatcaagatgatctggttgtcatgtttactgaatgctttgatgtcatatttgctggtggtccagaagaagttctaaaaaaattccaaaaggcaaaccacaaagtggtctttgcagcagatggaattttgtggccagataaaagactagcagacaagtatcctgttgtgcacattgggaaacgctatctgaattcaggaggatttattggctatgctccatatgtcaaccgtatagttcaacaatggaatctccaggataatgatgatgatcagctcttttacactaaagtttacattgatccactgaaaagggaagctattaacatcacattggatcacaaatgcaaaattttccagaccttaaatggagctgtagatgaagttgttttaaaatttgaaaatggcaaagccagagctaagaatacattttatgaaacattaccagtggcaattaatggaaatggacccaccaagattctcctgaattattttggaaactatgtacccaattcatggacacaggataatggctgcactctttgtgaattcgatacagtcgacttgtctgcagtagatgtccatccaaacgtatcaataggtgtttttattgagcaaccaaccccttttctacctcggtttctggacatattgttgacactggattacccaaaagaagcacttaaactttttattcataacaaagaagtttatcatgaaaaggacatcaaggtattttttgataaagctaagcatgaaatcaaaactataaaaatagtaggaccagaagaaaatctaagtcaagcggaagccagaaacatgggaatggacttttgccgtcaggatgaaaagtgtgattattactttagtgtggatgcagatgttgttttgacaaatccaaggactttaaaaattttgattgaacaaaacagaaagatcattgctcctcttgtaactcgtcatggaaagctgtggtccaatttctggggagcattgagtcctgatggatactatgcacgatctgaagattatgtggatattgttcaagggaatagagtaggagtatggaatgtcccatatatggctaatgtgtacttaattaaaggaaagacactccgatcagagatgaatgaaaggaactattttgttcgtgataaactggatcctgatatggctctttgccgaaatgctagagaaatgactttacaaagggaaaaagactcccctactccggaaacattccaaatgctcagccccccaaagggtgtatttatgtacatttctaatagacatgaatttggaaggctattatccactgctaattacaatacttcccattataacaatgacctctggcagatttttgaaaatcctgtggactggaaggaaaagtatataaaccgtgattattcaaagattttcactgaaaatatagttgaacagccctgtccagatgtcttttggttccccatattttctgaaaaagcctgtgatgaattggtagaagaaatggaacattacggcaaatggtctgggggaaaacatcatgatagccgtatatctggtggttatgaaaatgtcccaactgatgatatccacatgaagcaagttgatctggagaatgtatggcttcattttatccgggagttcattgcaccagttacactgaaggtctttgcaggctattatacgaagggatttgcactactgaattttgtagtaaaatactcccctgaacgacagcgttctcttcgtcctcatcatgatgcttctacatttaccataaacattgcacttaataacgtgggagaagactttcagggaggtggttgcaaatttctaaggtacaattgctctattgagtcaccacgaaaaggctggagcttcatgcatcctgggagactcacacatttgcatgaaggacttcctgttaaaaatggaacaagatacattgcagtgtcatttatagatccctaagttatttacttttcattgaattgaaatttattttggatgaatgactggcatgaacacgtctttgaagttgtggctgagaagatgagaggaatatttaaataacatcaacagaacaacttcactttgggccaaacatttgaaaaactttttataaaaaattgtttgatatttcttaatgtctgctctgagccttaaaacacagattgaagaagaaaagaaagaaaaaacttaaatatttatttctatgctttgttgcctctgagaataatgacaatttatgaatttgtgtttcaaattgataaaatatttaggtacaaataacaagactaataatattttcttatttaaaaaaagcatgggaagatttttatttatcaaaatatagaggaaatgtagacaaaatggatataaatgaaaattaccatgttgtaaaaccttgaaaatcagattctaactggatttgtatgcaactaagtatttttctgaacacctatgcaggtcttatttacagtagttactaagggaacacacaaagaattacacaacgttttcctcaagaaaatggtacaaaacacaaccgaggagcgtatacagttgaaaacatttttgttttgattggaaggcagattattttatattagtattaaaaatcaaaccctatgtttctttcagatgaatcttccaaagtggattatattaagcaggtattagatttaggaaaacctttccatttcttaaagtattatcaagtgtcaagatcagcaagtgtccttaagtcaaacaggttttttttgttgttgtttttgctttgtttccttttttagaaagttctagaaaataggaaaacgaaaaatttcattgagatgagtagtgcatttaattattttttaaaaaactttttaagtacttgaattttatatcaggaaaacaaagttgttgagccttgcttcttccgttttgccctttgtctcgctccttattctttttttggggggagggttatttgcttttttatcttcctggcataatttccattttattcttctgagtgtctatgttaacttccctctatcccgcttataaaaaaattctccaacaaaaatacttgttgacttgatgttttatcacttctctaagtaaggttgaaatatccttattgtagctactgtttttaatgtaaaggttaaacttgaaaagaaattcttaatcacggtgccaaaattcattttctaacaccatgtgttagaaaattataaaaaataaaataattttagaaaaaaaaaaaaaaa

(19) United States
(12) Patent Application Publication
(10) Pub. No.: US 2020/0263250 A1
(43) Pub. Date: Aug. 20, 2020

(54) ACTIVITY-DEPENDENT GENE PAIRS AS THERAPEUTIC TARGETS AND METHODS AND DEVICES TO IDENTIFY THE SAME

(71) Applicant: Wayne State University, Detroit, MI (US)

(72) Inventors: Leonard Lipovich, Detroit, MI (US); Jeffrey A. Loeb, Beverly Hills, MI (US)

(73) Assignee: Wayne State University, Detroit, MI (US)

(21) Appl. No.: 16/826,010

(22) Filed: Mar. 20, 2020

Related U.S. Application Data
(62) Division of application No. 15/684,845, filed on Aug. 23, 2017.
Application No. 15/684,845 is a continuation of application No. 14/423,086, filed on Feb. 20, 2015, now abandoned.
(60) Provisional application No. 61/692,162, filed on Aug. 22, 2012.

(51) Int. Cl. C12Q 1/6876; C12N 15/113; C12Q 1/6883

(52) U.S. Cl. CPC C12Q 1/6876; C12N 15/113; C12Q 1/6883; C12Q 2600/112; C12Q 2600/136; C12N 2310/14; C12Q 2600/158

(57) ABSTRACT

Activity-dependent gene pairs as therapeutic targets and methods and devices to identify the same are provided. The methods and devices allow transcriptome-wide analysis of regulatory long non-coding RNAs (lncRNAs), matched with differentially expressed protein-coding genes (mRNAs) and/or with other lncRNAs. The described methods and devices allow analysis of these activity-dependent gene pairs as therapeutic targets in a number of clinical conditions associated with altered electrical brain activity, including epilepsy.

### CROSS-REFERENCE TO RELATED APPLICATIONS

This application is a divisional of co-pending U.S. patent application Ser. No. 15/684,845, filed Aug. 23, 2017, which is a continuation of U.S. patent application Ser. No. 14/423,086, filed Feb. 20, 2015, which is a U.S. National Phase of International Patent Application No. PCT/US2013/056242, filed Aug. 22, 2013, which claims priority to U.S. Provisional Patent Application No. 61/692,162, filed Aug. 22, 2012. The contents each of these listed applications is incorporated herein in their entirety.

### STATEMENT OF GOVERNMENT INTEREST

This invention was made with government support under grants R01NS045207, R01NS058802 awarded by NIH and NINDS, and 1R03DA026021-01 awarded by NIH and NIDA. The government has certain rights in the invention.

### SEQUENCE LISTING

The present application includes a Sequence Listing in electronic format as a .txt file titled “Sequence_Listing.txt”, which was created on Feb. 13, 2020, and which has a size of ˜128 KB. The contents of the .txt file are incorporated by reference herein.

### FIELD OF THE DISCLOSURE

Disclosed herein are activity-dependent gene pairs as therapeutic targets and methods and devices to identify the same. The methods and devices allow transcriptome-wide analysis of regulatory long non-coding RNAs (lncRNAs), matched with differentially expressed protein-coding genes (mRNAs) and/or with other lncRNAs. The described methods and devices allow analysis of these activity-dependent gene pairs as therapeutic targets in a number of clinical conditions associated with altered electrical brain activity, including epilepsy.

### BACKGROUND OF THE DISCLOSURE

Many of the body's functions are mediated by the actions of proteins within and between cells. Generally, for a protein to exert an effect, the cell that will use or secrete the protein must create it. To create a protein the cell first makes a copy of the protein's gene sequence in the nucleus of the cell. This copy of the gene sequence that encodes for the protein (called messenger RNA (mRNA)) leaves the nucleus and moves to a region of the cell containing ribosomes. Ribosomes read the sequence of the mRNA and create the protein for which it encodes. This process of new protein synthesis is known as translation.

Long non-coding RNAs (lncRNAs) are a newly discovered type of RNA that are generated by cells but do not encode any protein. Despite their new-found prominence in the transcriptome (the total set of all RNA molecules), most lncRNAs remain poorly understood.

### SUMMARY OF THE DISCLOSURE

The present disclosure provides activity-dependent gene pairs as therapeutic targets and methods and devices to identify the same.

Particularly, one embodiment includes a method for identifying putative therapeutic targets comprising obtaining a paired brain tissue sample from a live human wherein each member of the pair has a different level of electrical brain activity from the other member; identifying long non-protein-coding RNA (lncRNA) molecules (lncRNAs) and protein-coding messenger RNA (mRNA) molecules (mRNAs) that are differentially expressed between the members of each individual sample pair; linking a first differentially expressed lncRNA with a differentially expressed mRNA and/or a second differentially expressed lncRNA when the gene encoding the first differentially expressed lncRNA overlaps with, or is adjacent to, the gene encoding the differentially expressed mRNA and/or the gene encoding the differentially expressed second lncRNA along the human genome, thereby identifying an lncRNA/mRNA gene pair and/or an lncRNA/lncRNA gene pair as putative cis-encoded therapeutic targets; and/or linking a first differentially expressed lncRNA with a differentially expressed mRNA and/or with a second differentially expressed lncRNA when the differentially expressed first lncRNA and the differentially expressed mRNA and/or second lncRNA are encoded at different genomic loci, thereby identifying an lncRNA/mRNA gene pair and/or an lncRNA/lncRNA gene pair as putative trans-encoded therapeutic targets.

In another embodiment, the linking of differentially expressed lncRNA with differentially expressed mRNA and/or lncRNA further requires that the differential expression of the lncRNA and mRNA or lncRNA and lncRNA be observed in more than one brain sample pair, each pair having a low electrical brain activity member and a high electrical brain activity member.

In another embodiment, the electrical brain activity is classified as high or low based on the frequency and/or amplitude of interictal and/or ictal spiking.

In another embodiment, the differential expression is identified by quantifying lncRNA and mRNA expression. In another embodiment, the expression quantification utilizes at least one microarray capable of quantifying lncRNA expression and mRNA expression.

In another embodiment, the quantifying utilizes at least one microarray capable of quantifying lncRNA expression and at least one microarray capable of quantifying mRNA expression wherein consistency of differential expression data between the at least one lncRNA microarray and the at least one mRNA microarray is evaluated by correlating the fold-change of protein-coding control genes common to both arrays.

Another embodiment further comprises evaluating the putative therapeutic target as a site of effective intervention.

In another embodiment, the therapeutic target of a pair is lncRNA, mRNA and/or both.

Another embodiment includes a microarray for identifying putative therapeutic targets in the human brain comprising probes for lncRNA and probes for mRNA wherein at least a subset of the mRNA probes is included based on the representation of their corresponding genes by probes on a different genome wide expression analysis microarray.

In another embodiment, the lncRNA probes are 50-mer to 70-mer probes mapped to a single genomic location. In another embodiment, the lncRNA probes are free of interspersed and simple repeats and segmental duplications.

In another embodiment, the microarray comprises 7 or 8 distinct probes per lncRNA.

In another embodiment, the microarray comprises probes for at least 1000 lncRNA genes.

Another embodiment includes a method of assessing putative therapeutic targets in the human brain comprising exposing human neuroblastoma cells to either a single depolarization or repeated depolarizations; identifying time-dependent differential lncRNA and mRNA expression in the cells exposed to either single and/or repeated depolarizations, relative to untreated control cells; and (i) linking a first differentially expressed lncRNA with differentially expressed mRNAs and/or second differentially expressed lncRNAs when the gene encoding the first differentially expressed lncRNA overlaps with, or is adjacent to, the gene encoding the differentially expressed mRNA or second differentially expressed lncRNA thereby identifying lncRNA/mRNA and/or lncRNA/lncRNA gene pairs as putative cis-encoded therapeutic targets; and/or (ii) linking a first differentially expressed lncRNA with a differentially expressed mRNA and/or with a second differentially expressed lncRNA when the first lncRNA and mRNA and/or second lncRNA are encoded at different genomic loci, thereby identifying lncRNA/mRNA and/or lncRNA/lncRNA gene pairs as putative trans-encoded therapeutic targets.

Another embodiment includes a method comprising targeting the first lncRNA of an lncRNA/mRNA or lncRNA/lncRNA gene pair as a putative therapeutic target wherein the first lncRNA and mRNA or second lncRNA are differentially expressed in areas of the brain having a different characteristic demonstrated to be relevant in one or more of epileptic activity, inflammation, cellular proliferation multiple sclerosis, neurodegeneration or autism and/or have been linked because the gene encoding the differentially expressed lncRNA overlaps with, or is adjacent to, the gene encoding the differentially expressed mRNA or second differentially expressed lncRNA.

In another embodiment, the gene pair is BDNFOS (SEQ ID NO: 1)/BDNF (SEQ ID NO: 2); AF086035 (SEQ ID NO: 3)/MAPK1IP1L (SEQ ID NO: 4); AK093366 (SEQ ID NO: 5)/AG2 (SEQ ID NO: 6); BC047792 (SEQ ID NO: 7)/PURB (SEQ ID NO: 8); AK096235 (SEQ ID NO: 9)/LCP1 (SEQ ID NO: 10); AL110130 (SEQ ID NO: 11)/SMEK2 (SEQ ID NO: 12); BC013641 (SEQ ID NO: 13)/ARC (SEQ ID NO: 14); hTF27297 (SEQ ID NO: 15)/CYR61 (SEQ ID NO: 16); RPPH1 (SEQ ID NO: 17)/NEAT1 (SEQ ID NO: 18); NEAT1 (SEQ ID NO: 18)/EGR3 (SEQ ID NO: 19); NEAT1 (SEQ ID NO: 18)/CR600638 (SEQ ID NO: 20); RPPH1 (SEQ ID NO: 17)/NEAT2 (MALAT1) (SEQ ID NO: 21); CR600638 (SEQ ID NO: 20)/FLT1 (SEQ ID NO: 22); BC012463 (SEQ ID NO: 23)/LRRN1 (SEQ ID NO: 24); CR615000 (SEQ ID NO: 25)/ERAP1 (SEQ ID NO: 26); BCO28229 (SEQ ID NO: 27)/BTG3 (SEQ ID NO: 28); BC078172 (SEQ ID NO: 29)/VIM (SEQ ID NO: 30); AF075087 (SEQ ID NO: 31)/S1PR1 (SEQ ID NO: 32); AK123944 (SEQ ID NO: 33)/TRIM47 (SEQ ID NO: 34); or AL110176 (SEQ ID NO: 35)/PLOD2 (SEQ ID NO: 36).

Another embodiment includes a method comprising targeting an lncRNA gene as a site of putative therapeutic intervention wherein the lncRNA gene is differentially expressed in at least one area of the brain having a different characteristic then a second area and wherein the lncRNA gene is RPPH1; NEAT1 or NEAT2.

Another embodiment includes a method comprising targeting a gene as a site of therapeutic intervention when the gene was identified by a method or microarray disclosed herein. In another embodiment, the targeting includes gene silencing or gene activating. In another embodiment, the targeting includes gene silencing through RNA interference (RNAi).

### BRIEF DESCRIPTION OF THE FIGURES

FIGS. 1A-1C. Reciprocal pattern of BDNF and BDNFOS gene expression in electrically active human neocortex. FIG. 1A. Summary of human epilepsy patients showing the ratios of electrical discharges, regions of neocortex sampled for each, and histopathology. All tissue sampled for gene expression changes had a normal histological structure, even in the presence of nearby structural abnormalities. FIG. 1B. Long-term brain surface recordings obtained prior to tissue resection were used to differentiate electrode locations with high- and low-spiking interictal activities for each patient. FIG. 1C. While both the activity-dependent immediate early gene EGR1 and BDNF are both constitutively upregulated in high-spiking cortex, BNDFOS was consistently downregulated in the same samples. The downregulation was significant (p=0.016, Wilcoxon's test; BDNFOS fold change <−1.1, 95% CI), Bars represent average values for all 7 patients shown with the shading of the circles corresponding to patients shown in A.

FIGS. 2A and 2B. Downregulation of BDNFOS induces BDNF expression in Sy5Y cells. FIG. 2A. The BDNFOS/BDNF gene locus shows antisense overlap between BDNF and BDNFOS (UCSC Genome Browser). Three siRNAs were generated from a non-overlapping BDNFOS exon (positions 231-305 of SEQ ID NO: 1 shown; s1 (which binds to positions 231-255 of SEQ ID NO: 1), s2 (which binds to positions 256-281 of SEQ ID NO: 1), s3 (which binds to positions 283-305 of SEQ ID NO: 1)). FIG. 2B. Downregulation of BDNFOS lncRNA using each of these siRNAs produced a corresponding increase in BDNF mRNA levels at 24 h and 48 h. Expression level changes are relative to a mock-electroporation negative control. Standard error bars displayed. No further BDNFOS knockdown or BDNF rescue persisted at the 48 h time point for s1 and s3 (data not shown).

FIGS. 3A and 3B. Genome-wide analysis of human cortex reveals activity-dependent gene pairs and standalone lncRNAs. FIG. 3A. This experimental design of paired high- and low-spiking brain samples from the 7 patients shown in FIG. 1a was used both to interrogate coding and non-coding gene transcription as a function of brain activity. A dye-flip, quadruplicate microarray design was used with both a genome-wide coding array and a custom lncRNA array encompassing 5586 lncRNA genes with 7 probes/gene. Based on a rigorous statistical cutoff, a total of 4044 protein-coding and 1288 lncRNA genes were initially identified for these 7 patients (>1.4 fold and FDR<5% for each probe). LncRNA genes were further subdivided based on known cis-antisense partners of mRNAs, lncRNAs located <10 kb from any known gene, or standalone lncRNAs >10 kb from any known gene. Due to gene chains, some lncRNAs belonged simultaneously to the first two of these three categories. FIG. 3B. Pairs of differentially expressed mRNA and lncRNA genes that were either in an antisense overlap or <10 kb neighbors (which we define as “cis-encoded pairs”) are shown together with lncRNAs that reside at other types of genomic loci. Many of the affected mRNAs have known functions in synaptic plasticity. The arrows to the left of the gene/pairs have been validated by qPCR. *BDNFOS/BDNF was discovered by targeted qPCR and did not meet statistical significance on the microarrays. Note that since the creation of this figure, some “standalone” lncRNAs have been paired through additional analysis. To date these lncRNAs include RPPH1, NEAT1 and NEAT2 (MALAT1).

FIG. 4. Parallel patterns of activity-dependent gene pairs. As a means to identify activity-dependent lncRNAs with potential roles in synaptic plasticity, the expression patterns of 13 known activity-dependent coding genes against the entire dataset of lncRNAs were probed for parallel patterns of expression. This figure shows significant relationships between these 13 genes and 26 lncRNAs identified using an R>0.90 cutoff. Each line represents a significant correlation and the proximity of the genes is directly proportional to this significance. The length of each line is inversely proportional to the correlation coefficient that is based on the average of correlations from probes above the 0.90 cutoff. The width of each line is directly proportional to the number of probes above the 0.90 cutoff. Coding genes are shown in dark grey while lncRNAs are in lighter grey. This figure was prepared using Cytoscape (available on the World Wide Web at cytoscape.org).

FIGS. 5A-5C. Repeated depolarization in vitro can replicate patterns of coding/non-coding gene transcription. FIG. 5A. While transient CREB phosphorylation is induced with a single depolarization of Sy5Ycells with 100 mM KCl producing downregulation of BDNF and BDNFOS, FIG. 5B. repeated depolarization produces more sustained CREB activation and results in a reciprocal pattern of BDNF/BDNFOS transcription at 24 hours (shown by the brackets). For each of these, the middle panel shows a quantitation of triplicate Western blots as well as triplicate qPCR results for EGR1 to show activity dependent transcription, together with BDNF and BDNFOS at each time point. FIG. 5C. The expression of three cis-encoded lncRNA/mRNA pairs and one known functional lncRNA (NEAT1) was examined in the same SY5Y repeated depolarization time course as in (b) showing widely different patterns of expression of lncRNAs located both within and outside of genomic loci that harbor cis-encoded lncRNA/mRNA pairs (as used herein, “cis-encoded” includes situations where an lncRNA and an mRNA or an lncRNA and an lncRNA are expressed from the same or adjacent genomic loci. Adjacent genomic loci include those with end nucleotides within 10 nucleotides of the other).

FIG. 6. Genomic complexity of the human AK093366/AG2 lncRNA/mRNA cis-antisense pair which is co-differentially expressed in human neocortical epilepsy.

FIGS. 7A-7G. Taqman qRTPCR results closely parallel microarray results for lncRNA and mRNA differential expression at lncRNA/mRNA cis-pairs across the within-patient sample pairs of high- and low-activity neocortical regions. MALAT-1 is not shown because of the discrepancy between its microarray probeset coverage and its Taqman amplicons coverage.

### DETAILED DESCRIPTION

The present disclosure provides activity-dependent gene pairs as therapeutic targets and methods and devices to identify the same. The present disclosure also describes the first genome-wide analysis of human brain long non-coding RNA (lncRNA)-based gene pairs as a function of coding mRNA or other lncRNA pairings and electrical brain activity. Many of the coding mRNAs identified in this way are known to modulate activity-dependent gene expression in the human brain, suggesting that these particular lncRNA/mRNA pairs form regulatory networks related to human brain plasticity. LncRNA/lncRNA pairs showing differential expression between areas of high and low brain activity were also observed. The described lncRNA/mRNA and lncRNA/lncRNA pairs provide targets for rational therapeutic development in a number of disorders and diseases associated with differential or perturbed electrical brain activity including epilepsy. As used herein, “differential expression”, “significantly differentially expressed” and similar terms mean that expression of a gene is significantly different based on a statistical power analysis, the results of which can be validated by qPCR at a 95% confidence interval. “Expression” as used herein includes (i) transcription of a gene encoding lncRNA and (ii) transcription of a gene encoding protein-encoding RNA and/or translation of the protein-coding RNA. “High” and “low” electrical brain activity, interictal and/or ictal spiking as used herein are relative terms to be compared between brain areas in a given patient during a given procedure.

The abundance of non-protein-coding transcriptional units rivals the numbers of known mRNAs. LncRNA genes can be defined by four criteria: encoding transcripts that lack any open reading frames (ORFs) greater than 100 amino acids or possessing protein database homologies; being within the known range of lengths of mammalian mRNAs (from ˜300 nt to >20,000 nt in length); support by transcript-to-genome alignments from cDNA data; and absence of matches to any known non-coding-RNA classes. Further information regarding lncRNAs may be found by consulting the GENCODE Project which annotates evidence-based gene features of the human genome including protein-coding loci with alternatively-transcribed variants, non-coding loci with transcript evidence and pseudogenes. GENCODE has assigned the lncRNA biotype to certain genes and transcripts, signifying that those genes and transcripts encode and represent, respectively, lncRNAs based on the best available manual-annotation and experimental data. Any gene or transcript given an lncRNA biotype by GENCODE is an lncRNA as the term is used herein regardless of whether the particular gene or transcript meets the four criteria provided above. The GENCODE gene sets are used by the ENCODE consortium.

Numerous lncRNAs are transcribed in the vicinity of known mRNAs, and regulate those known genes through epigenetic mechanisms. Functionally, lncRNAs can have regulatory effects on coding mRNAs through a number of mechanisms that include cis-antisense lncRNA transcripts that repress their sense-strand protein-coding partners. LncRNAs can also enhance expression of differentially expressed gene pair partners. LncRNAs encoded in an antisense orientation to, and overlapping with, known mRNAs are particularly abundant. The vast majority of these lncRNAs remain devoid of known functions.

The human brain is composed of a diverse set of cell types connected through complex synaptic arrangements. The degree of synaptic activity in the brain can be translated into functional and structural changes through activity-dependent changes in gene expression. Although these changes can be effected through direct activation of synaptic genes, they can also be achieved through the release of neurotrophic factors such as brain-derived neurotrophic factor (BDNF) that have direct effects on synaptic architecture and indirect effects by producing changes in gene expression. BDNF, a member of the nerve growth factor family, regulates the survival and differentiation of neuronal populations, axonal growth and pathfinding, dendritic growth and morphology and has been linked to many human brain disorders. BDNF mRNA and protein are upregulated by seizure activity in animal models of epilepsy as well as in human brain tissues that display increased epileptic activities. The genomic locus encoding BDNF is structurally complex and also encodes BDNFOS, a primate-specific lncRNA that is antisense to the coding BDNF gene. BDNF and BDNFOS form double-stranded duplexes, suggesting a potential for BDNFOS to post-transcriptionally regulate BDNF.

BDNF binding to its receptors results in a diverse array of downstream signaling pathways including the activation of cyclic adenosine monophosphate response element binding protein (CREB), that in turn can also regulate BDNF by binding to a cognate site within the BDNF gene. Activation of CREB by phosphorylation at Serine 106 as a result of neuronal activity leads to changes in gene expression that cause reinforcement and stabilization of more active neuronal circuits. Downstream from phosphorylated CREB (pCREB), immediate early genes (IEGs) have been shown to mediate long-lasting changes in neuronal structure and excitability. Upstream of CREB activation, several known signaling pathways are rapidly activated in response to neuronal activity, including CaMKinase IV, protein kinase A, and MAPK. A pattern of transcriptional activation in human brain regions where seizures start strongly implicates sustained MAPK/CREB activation and downstream coding gene activations that could underlie layer specific changes in synaptic architecture that makes these regions prone to seizures.

Given that human lncRNA genes tend to be less well-conserved than mRNAs, and have unique transcripts not found in other species, a uniquely human system to examine activity-dependent gene expression for both coding and non-coding RNAs using a pairwise comparison of human cortical regions displaying variable degrees of epileptic activities was sought. These brain regions were removed as part of surgical treatment for intractable seizures. It was shown that regions of human neocortex that display increased activity and BDNF expression have reduced BDNFOS expression and that BDNFOS directly down-regulates BDNF in vitro. A custom microarray platform to perform a transcriptome-wide analysis of other regulatory lncRNAs was also developed and matched to differentially expressed mRNAs or other lncRNAs to develop a genome-wide list of lncRNA/mRNA gene pairs and/or lncRNA/lncRNA gene pairs. Many of the coding mRNAs identified in this way are known to modulate activity-dependent gene expression in the human brain, suggesting that these lncRNA/mRNA pairs form a newly revealed regulatory network of human brain plasticity. Identified lncRNA/lncRNA pairs also provide targets for potential therapeutic intervention.

Genome-Wide Integration of Activity-Dependent Gene Pairs as a Function of Human Brain Activity.

The present disclosure describes a stringently filtered catalog of human lncRNAs and the genomic positional relationships between these lncRNAs and mRNAs and/or other lncRNAs, providing insights into lncRNA functions (see JIA et al. 2010, incorporated by reference for its teachings regarding the same). Despite their prominence in the transcriptome, most lncRNAs remain poorly understood. The present disclosure describes development of a custom lncRNA microarray to provide the first genome-wide analysis of human brain lncRNA-based gene pairs as a function of electrical brain activity. Several identified co-expressed lncRNA/mRNA gene pairs have important roles in activity-dependent synaptic plasticity either directly, such as BDNF and others involved in the MAPK/CREB signaling, or indirectly through the expression of regulatory lncRNAs such as MALAT-1 which are members of trans-encoded lncRNA/lncRNA gene pairs (e.g. RPPH1/MALAT-1, whereas MALAT-1 is an RNA-processing target of RPPH1).

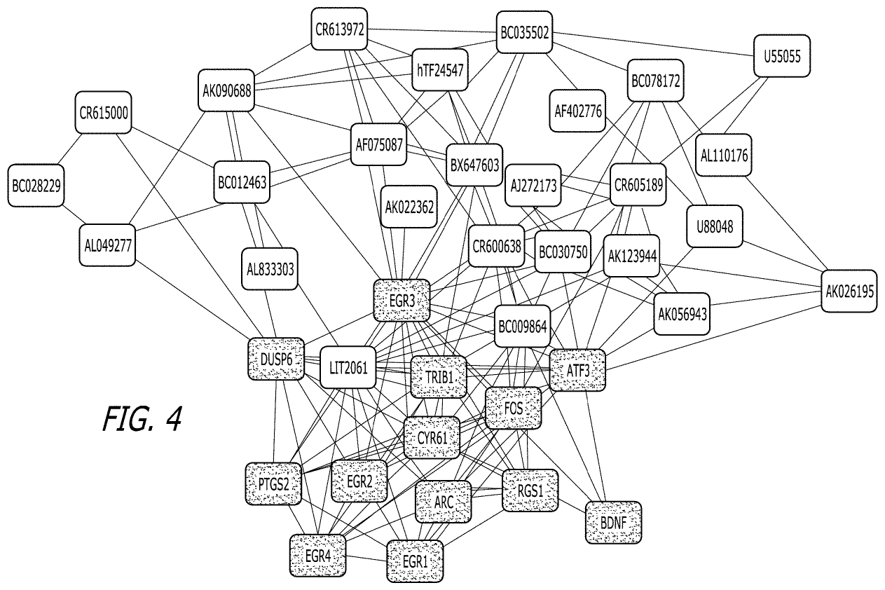

The described genome wide lncRNA expression survey of electrically active human neocortex uncovered hundreds of lncRNAs differentially expressed between more and less electrophysiologically active areas of the human neocortex. Of these lncRNAs, 26 were initially identified as expressed directly in proportion to known activity-dependent genes (FIG. 4). These lncRNAs represent therapeutic targets for human brain diseases, such as epilepsy. Without being bound by theory, the co-expression clustering topology (FIG. 4) suggests a network where mRNAs and lncRNAs are linked by previously uncharacterized lncRNA nodes (such as BC009864) as hubs with spoke edges extending simultaneously to multiple mRNAs and other lncRNAs. Eight lncRNA/mRNA cis-antisense and neighbor-gene pairs characterized by coordinated differential expression of both genes in each lncRNA/mRNA pair were also initially observed, suggesting lncRNA-mediated regulation of protein-coding gene expression in the human brain. These pairs also suggest that some mRNAs function at the RNA level to regulate lncRNA expression or in bidirectionally regulated feedback loops in cis. Other lncRNAs such as NEAT1 were detected only by the trans-regulation analysis, which targeted lncRNAs whose expression was highly correlated with mRNAs or other lncRNAs regardless of the genomic mapping location of those coding genes. The trans-regulation analysis implies NEAT1, the lncRNA from nuclear paraspeckles that is encoded near the NEAT2 locus, in regulatory interactions with activity-dependent genes in the brain. Three lines of evidence for activity-dependent NEAT1 function in the neocortex include (1) detection of NEAT1 as a differentially expressed lncRNA on the custom microarray analysis of human brain samples, (2) demonstration of activity-dependent NEAT1 expression in depolarized human SY5Y cell culture, and (3) the assignment of NEAT1 as a central node to a trans-encoded co-expression cluster of specific coding and non-coding RNAs (FIG. 4). The described cis-regulation and trans-regulation analyses uncovered different, nonredundant sets of lncRNAs, suggesting that specific lncRNAs are involved in both types of regulation, which for any given lncRNA may be mutually exclusive. These results represent the first functional evidence for a remarkably diverse pattern of lncRNA expression and function in the human brain.

Functionality of Activity-Dependent Gene Pairs in the Human Brain.

The described microarray results and qPCR analysis of both the epilepsy patient samples and the recurrent-depolarization Sh-sy5y cell culture time course, jointly represent the first demonstration that known lncRNAs are activity-dependent both in vivo and in cell culture. The complex, but similar, pattern of lncRNA/mRNA or lncRNA/lncRNA expression in activated human brain and in a chronically depolarized human neuronal cell line enables the temporal characterization of these regulatory gene pairs and provides a new system in which to assess these complex, primate-specific transcriptional gene pairs.

The present disclosure describes upregulation of three nuclear RNAs, RNase P (RPPH1), NEAT1, and MALAT-1, in high-activity areas of the neocortex. The catalytic-RNA component of RPPH1 is essential for the 3′ end cleavage of both NEAT1 and NEAT2/MALAT-1. Therefore, these three lncRNAs may comprise an lncRNA-mediated lncRNA maturation network in highly active brain regions. The function of this induced network is predicted to modulate the expression of synaptic genes, such as those whose mRNA levels are regulated by NEAT2/MALAT-1. This RNA-mediated regulatory network is also predicted to function either independently from, or synergistically with, the MAPK/pCREB pathway to regulate activity-dependent gene expression.

Ribonucleoprotein complexes that enable lncRNA function and complexes which facilitate lncRNA-mediated regulation of mRNAs in sense-antisense pairs can be identified by affinity columns and mass spectrometric analysis. This identification will allow therapeutic targeting of lncRNA/mRNA and/or lncRNA/lncRNA gene pairs.

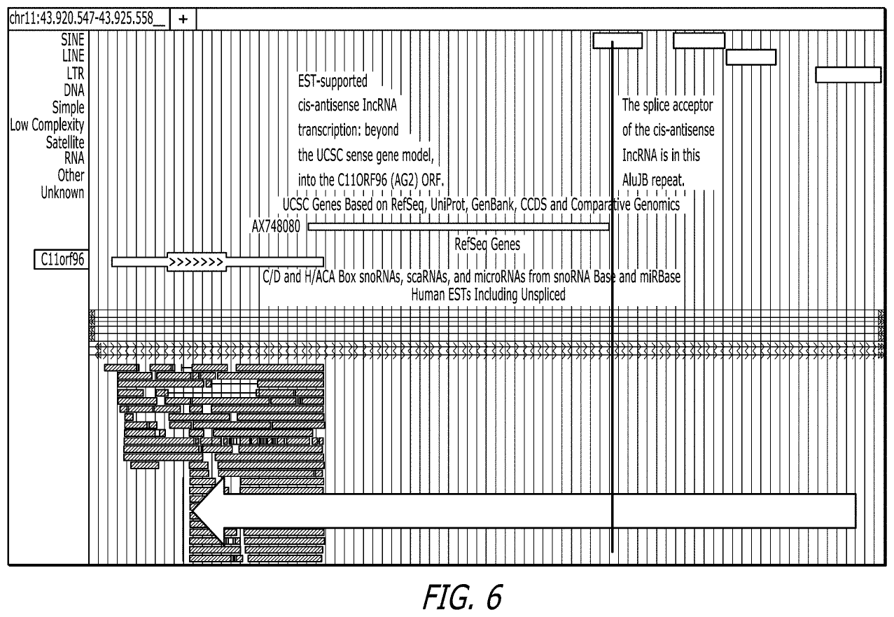

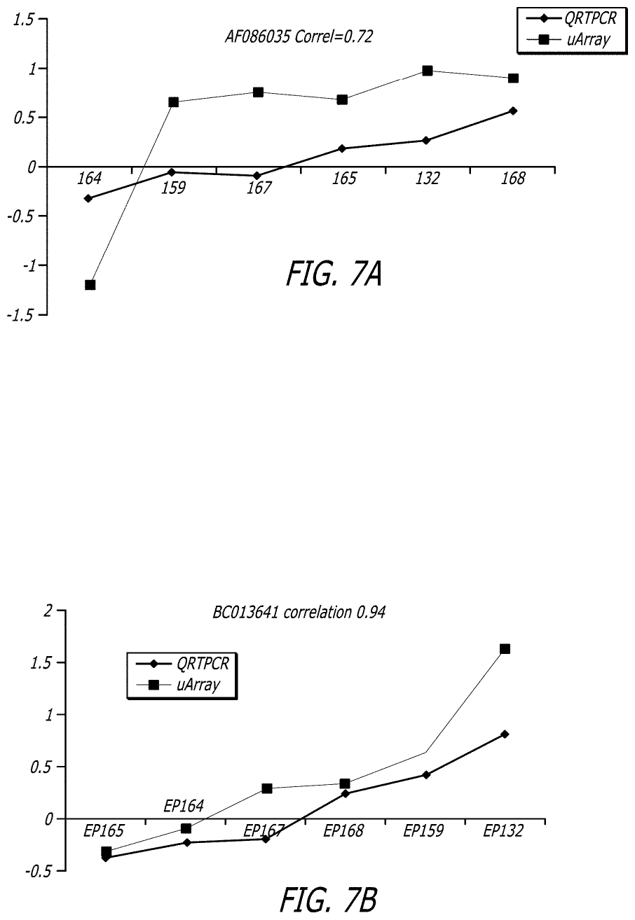

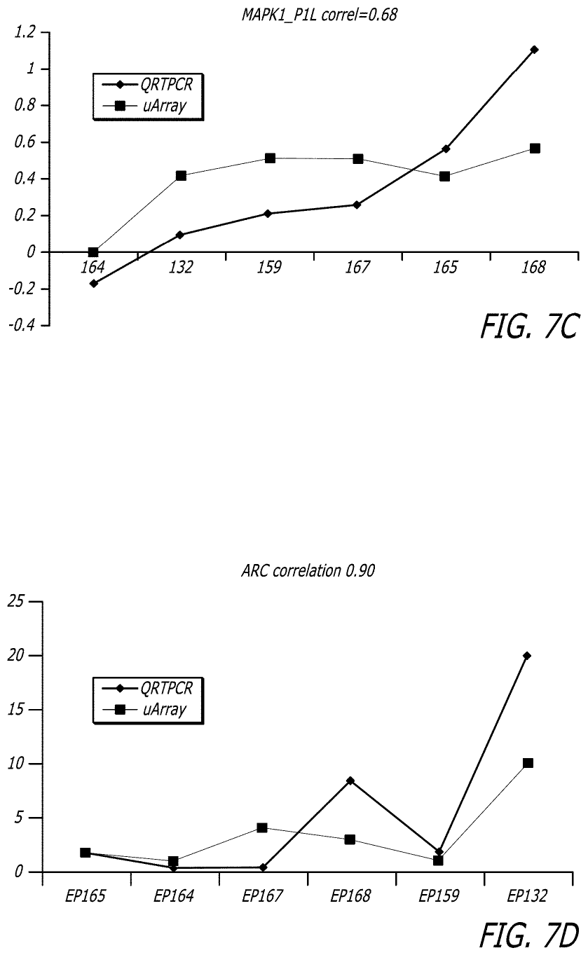

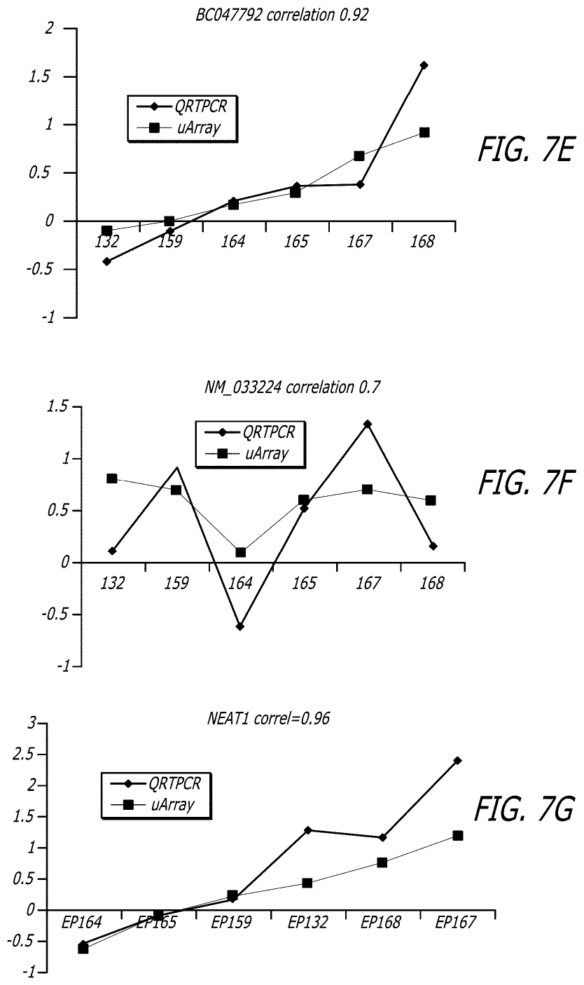

The genomic complexity of the human AK093366/AG2 lncRNA/mRNA cis-antisense pair (FIG. 6) is reminiscent of that observed in the BDNFOS/BDNF lncRNA/mRNA cis-antisense pair. Both the BDNFOS lncRNA gene and the AK093366 lncRNA gene contain primate-specific splice sites. The splice donor of AK093366's sole intron is primate-specific because it is harbored within an AluJb repeat. Alu repeats are the best-known class of primate-specific interspersed repeats and therefore key gene structure elements, including splice sites, contained within Alu repeats provide direct evidence that the corresponding gene structures either arose or were modified after the mammalian radiation, specifically in the primate lineage. Notably, EST-supported cis-antisense lncRNA transcription of the Alu-containing AK093366 transcriptional unit extends substantially beyond the UCSC C11ORF96 (AG2) gene model, and well into the C11ORF96 ORF. This underscores the utility of EST data, much of which remains unincorporated into reference gene models and annotations, in delineating the boundaries of lncRNA genes, including those involved in putative regulatory relationships with protein-coding counterparts. While Alu-containing lncRNAs have been implicated in the in-trans post-transcriptional regulation of gene expression via effecting mRNA decay, the currently described analysis suggests distinct, cis-regulatory roles in overlapping-gene regulation for certain Alu-containing lncRNAs, specifically AK093366. Two co-differentially-expressed lncRNA/mRNA cis-antisense pairs in the human neocortex, BDNFOS/BDNF and AK093366/AG2, thus feature primate-specific sequence at lncRNA gene splice junctions. mRNAs BDNF and AG2 are overlapped by endogenous antisense lncRNAs containing exonic Alu repeats, and these gene pairs are codifferentially-expressed in active areas of the human epileptic neocortex.

BDNFOS-mediated regulation of BDNF provides evidence that primate-specific regulation of conserved mRNAs by cis-antisense lncRNAs takes place in epilepsy, a human brain disorder. The co-expression of mRNAs and non-conserved lncRNAs at loci other than BDNF, including AG2, demonstrates that primate-specific regulation of conserved genes by nonconserved lncRNAs in the human brain is not unique to the BDNF locus.

A Primate-Specific lncRNA Regulatory Mechanism for BDNF.

A striking feature of the BDNF/BDNFOS locus is the complexity of its genomic landscape, which is highly representative of the genomic properties observed at lncRNA-encoding loci throughout mammalian genomes. Human BDNFOS is part of a three-gene genomic positional chain: it shares a putative bidirectional promoter with the LIN7C gene at its 5′ end, while sharing its exonic cis-antisense overlap with BDNF exonic sequences at the 3′ end. In addition, BDNFOS may have emerged in recent mammalian evolution, after the primate-rodent divergence. A possible recent origin for this lncRNA gene is supported by two lines of evidence. First, several splice sites of BDNFOS are poorly conserved outside of primates, suggesting that the genomic structure of BDNFOS either arose or was modified specifically in primate evolution. This is consistent with the recent finding that lncRNA genes may comprise a majority of primate-specific genes. Second, there is no evidence for a BDNFOS-like gene between mouse Lin7c and mouse BDNF in mouse cDNA and EST sequence data (UCSC transcript-to-genome alignments). This genomic and evolutionary complexity of the BDNF/BDNFOS locus, implies that functional lncRNAs in the human brain may be characterized by interspecies non-conservation or high divergence of their gene structures. This is of particular interest because of the persistent inverse co-differential-expression of the BDNF/BDNFOS gene pair as a function of human brain activity shown here together with the observed increase in BDNF mRNA levels following knock down of BDNFOS. The present disclosure therefore provides a uniquely human view of activity-dependent gene pairs in the brain whose endogenous components cannot be modeled in rodents or other non-primate species. A set of differentially expressed miRNAs, including miR-30a-5p, act as post-transcriptional inhibitors of BDNF in prefrontal cortex. The demonstration of the primate-specific lncRNA BDNFOS as a post-transcriptional inhibitor of BDNF complements this miRNA work, suggesting that BDNF is targeted by multiple RNA-mediated regulatory mechanisms involving short and long, ancient as well as evolutionarily young ncRNAs.

The methods and devices described herein identified the following lncRNA/mRNA or lncRNA/lncRNA gene pairs as activity-dependent targets for therapeutic intervention (lncRNA/mRNA or lncRNA/lncRNA): BDNFOS (SEQ ID NO: 1)/BDNF (SEQ ID NO: 2); AF086035 (SEQ ID NO: 3)/MAPK1IP1L (SEQ ID NO: 4); AK093366 (SEQ ID NO: 5)/AG2 (SEQ ID NO: 6); BC047792 (SEQ ID NO: 7)/PURB (SEQ ID NO: 8); AK096235 (SEQ ID NO: 9)/LCP1 (SEQ ID NO: 10); AL110130 (SEQ ID NO: 11)/SMEK2 (SEQ ID NO: 12); BC013641 (SEQ ID NO: 13)/ARC (SEQ ID NO: 14); hTF27297 (SEQ ID NO: 15)/CYR61 (SEQ ID NO: 16); RPPH1 (SEQ ID NO: 17)/NEAT1 (SEQ ID NO: 18); NEAT1 (SEQ ID NO: 18)/EGR3 (SEQ ID NO: 19); NEAT1 (SEQ ID NO: 18)/CR600638 (SEQ ID NO: 20); RPPH1 (SEQ ID NO: 17)/NEAT2 (MALAT1) (SEQ ID NO: 21); CR600638 (SEQ ID NO: 20)/FLT1 (SEQ ID NO: 22); BC012463 (SEQ ID NO: 23)/LRRN1 (SEQ ID NO: 24); CR615000 (SEQ ID NO: 25)/ERAP1 (SEQ ID NO: 26); BCO28229 (SEQ ID NO: 27)/BTG3 (SEQ ID NO: 28); BC078172 (SEQ ID NO: 29)/VIM (SEQ ID NO: 30); AF075087 (SEQ ID NO: 31)/S1PR1 (SEQ ID NO: 32); AK123944 (SEQ ID NO: 33)/TRIM47 (SEQ ID NO: 34); and AL110176 (SEQ ID NO: 35)/PLOD2 (SEQ ID NO: 36).

In a preferred embodiment, the first lncRNA of a pair is targeted to effect therapeutic intervention. In alternative embodiments, the mRNA and/or the second lncRNA of a pair can be targeted. In further embodiments, the first lncRNA and mRNA or second lncRNA of a pair can both be targeted.

The genes of the above pairs to target include the sequences above as well the reverse complements thereof and allelic variants. Allelic variants include slightly different sequences that originate from the same chromosomal position or the same position on an allelic chromosome. Therapeutic targets as disclosed herein also include sequences with at least 90% sequence identity; at least 91% sequence identity; at least 92% sequence identity; at least 93% sequence identity, at least 94% sequence identity, at least 95% sequence identity, at least 96% sequence identity, at least 97% sequence identity, at least 98% sequence identity or at least 99% sequence identity to SEQ ID NO. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 and/or 36. Percentage of sequence identity is determined by comparing two optimally aligned sequences (e.g., nucleic acid sequences) over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleotide or amino acid residue occurs in both sequences to yield the number of matched positions, dividing the number of matched positions by the total number of positions in the comparison window, and multiplying the result by 100 to yield the percentage of sequence identity. A sequence that is identical at every position in comparison to a reference sequence is said to be 100% identical to the reference sequence, and vice-versa. Therapeutic targets as disclosed herein also include those sequences that hybridize to one or more of SEQ ID NO. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 and/or 36 under high stringency conditions. As used herein, high stringency conditions include hybridization in SXSSC buffer at 65° C. for 16 hours; wash twice in 2×SSC buffer at room temperature for 15 minutes each; and wash twice in 0.5×SSC buffer at 65° C. for 20 minutes each.

As will be understood by one of ordinary skill in the art, the therapeutic targets disclosed herein can be targeted through any mechanism capable of increasing or decreasing their function, as appropriate. Many of these strategies will be based on complementary binding properties, but the present disclosure is not so limited and can include any form of effective intervention, however formed and delivered. The currently-disclosed targets can also be targeted by inhibiting or increasing the activity of upstream or downstream molecules, including those described herein in relation to particular targets including CREB, CaM Kinase IV, protein kinase A and MAPK. While not limiting the foregoing inclusive statements in any manner, the following description provides several appropriate targeting strategies.

One targeting approach can include gene silencing. This approach refers to the reduction in transcription, translation, expression or activity of a nucleic acid, as measured by transcription level, mRNA or lncRNA level, enzymatic activity, methylation state, chromatin state or configuration, translational level, or other measure of its activity or state in a cell or biological system. Such activities or states can be assayed directly or indirectly. Gene silencing also includes the reduction or amelioration of activity associated with a nucleic acid sequence, such as its ability to function as a regulatory sequence, its ability to be transcribed, its ability to be translated and result in expression of a protein, regardless of the mechanism whereby such silencing occurs.

One method of gene silencing includes RNA interference (RNAi). RNAi refers to the process by which a polynucleotide or double stranded polynucleotide comprising at least one ribonucleotide unit exerts an effect on a biological process through disruption of gene expression. The process includes but is not limited to gene silencing by degrading mRNA, interactions with tRNA, rRNA, hnRNA, cDNA and genomic DNA, as well as methylation of DNA and ancillary proteins.

Another targeting approach can include gene activating. This approach refers to an increase in transcription, translation, expression or activity of a nucleic acid, as measured by transcription level, mRNA or lncRNA level, enzymatic activity, methylation state, chromatin state or configuration, translational level, or other measure of its activity or state in a cell or biological system. Such activities or states can be assayed directly or indirectly. Furthermore, gene activating includes the increase of activity associated with a nucleic acid sequence, such as its ability to function as a regulatory sequence, its ability to be transcribed, its ability to be translated and result in expression of a protein, regardless of the mechanism whereby such activation occurs.

Any type of nucleic acid capable of achieving gene silencing or gene activation can be used whether such nucleic acids are endogenously, exogenously and/or recombinantly-derived. Exemplary types of nucleic acid molecules that can be used in targeting strategies disclosed herein include:

Short interfering RNA (siRNA)—a double stranded nucleic acid that is capable of performing RNAi and that is between 18 and 30 base pairs in length. siRNAs can be duplexes, and can also comprise short hairpin RNAs, RNAs with loops as long as, for example, 4 to 23 or more nucleotides, RNAs with stem loop bulges, micro-RNAs, and short temporal RNAs. RNAs having loops or hairpin loops can include structures where the loops are connected to the stem by linkers such as flexible linkers. Flexible linkers can be comprised of a wide variety of chemical structures, as long as they are of sufficient length and materials to enable effective intramolecular hybridization of the stem elements. Typically, the length to be spanned is at least about 10-24 atoms. siRNAs can be endogenous or exogenous, although in practice, therapeutic siRNA will be exogenous.

MicroRNA (miRNA)—18-25 nucleotide non-coding RNAs derived from endogenous genes. MiRNAs assemble in complexes and recognize their targets by antisense complementarity. If the miRNA matches its target with 100% sequence identity, the target RNA is cleaved, and the miRNA acts like a siRNA. If the sequence identity is less than 100%, the translation of target RNA is blocked.

Small nucleolar RNAs (snoRNAs)—small RNA molecules that guide chemical modifications (methylation or pseudouridylation) of ribosomal RNAs (rRNAs) and other RNA genes (tRNAs and other small nuclear RNAs (snRNAs)).

As provided, a variety of types of nucleic acid molecules can be used to increase or decrease the function of the therapeutic targets identified herein. The nucleic acids can include either or both naturally occurring and modified nucleotides linked together by naturally occurring and/or non-naturally occurring nucleotide linkages. Nucleic acid molecules may be modified chemically or biochemically, or may contain non-natural or derivatized nucleotide bases. Such modifications include, for example, labels, methylation, substitution of one or more of the naturally occurring nucleotides with an analog, internucleotide modifications (e.g., uncharged linkages: for example, methyl phosphonates, phosphotriesters, phosphoramidates, carbamates, etc.; charged linkages: for example, phosphorothioates, phosphorodithioates, etc.; pendent moieties: for example, peptides; intercalators: for example, acridine, psoralen, etc.; chelators; alkylators; and modified linkages: for example, alpha anomeric nucleic acids, etc.). Additionally, the nucleic acid molecules may be modified at either the 3′ and/or 5′ end by any type of modification known in the art. For example, either or both ends may be capped with a protecting group, attached to a flexible linking group, attached to a reactive group to aid in attachment to a substrate, etc. The term “nucleic acid molecule” also includes any topological conformation, including single-stranded, double-stranded, partially duplexed, triplexed, hairpinned, circular, and padlocked conformations.

Conjugates may also be used to target the gene pairs described herein. As used herein, conjugates refer to molecules with at least two discrete components. In one embodiment, one component alters the physical properties of another component. The altered physical property can be, without limitation, shelf-life stability, in vivo half-life or cellular uptake. In a particular embodiment, the two components are covalently or non-covalently associated. In another embodiment, one component is a nucleic acid molecule and the second component is a non-nucleotide region such as, without limitation, polyethylene glycol, one or more fatty acid chains, one or more sugar residues, etc.

Pharmaceutically acceptable excipients such as vehicles, adjuvants, carriers and/or diluents can be used as appropriate Other components such as pH adjusting and buffering agents, tonicity adjusting agents, stabilizers, wetting agents and the like can also be used.

### EXAMPLES

### Experimental Rationales, Materials and Methods

Human Brain Tissue:

Patients who fail to respond to medical management of their seizures can greatly benefit from a 2-stage surgical procedure where long-term in vivo brain surface recordings are used to identify and remove epileptic brain regions. While removing seizure onset regions is key to a good outcome for improved seizure control, seizures from these brain regions are relatively infrequent compared to the small, but extremely frequent ‘interictal’ epileptic discharges that can occur almost constantly between seizures in some brain regions. In fact, several of the genes induced at seizure onset zones correlate precisely with interictal spiking rather than seizure frequency, suggesting that interictal spiking may be the driving force behind this altered expression pattern. Consistently, an animal model of interictal spiking without seizures was sufficient to produce neuronal layer-specific changes in these genes. The studies of the present disclosure have focused on brain regions with different levels of brain activity as measured by interictal spiking to identify the relationships between coding and non-coding or non-coding and non-coding transcripts in the human brain.

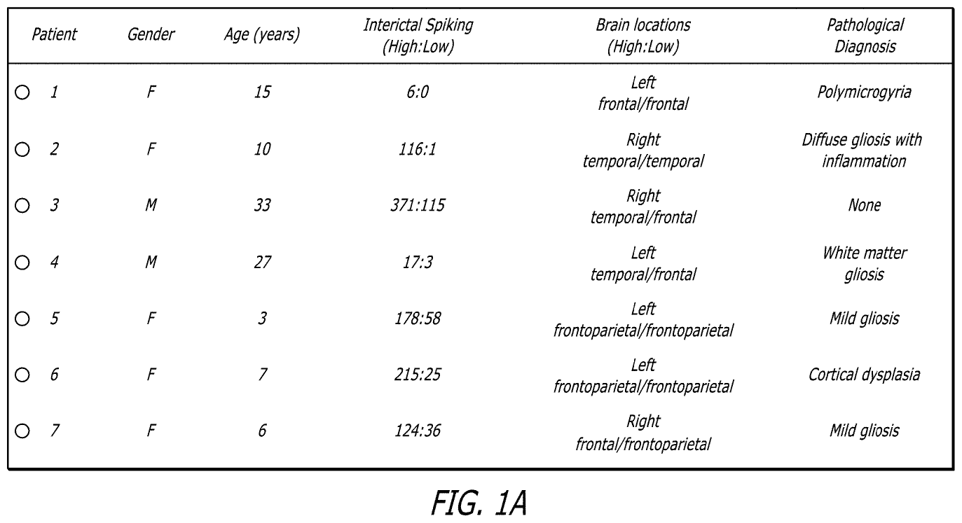

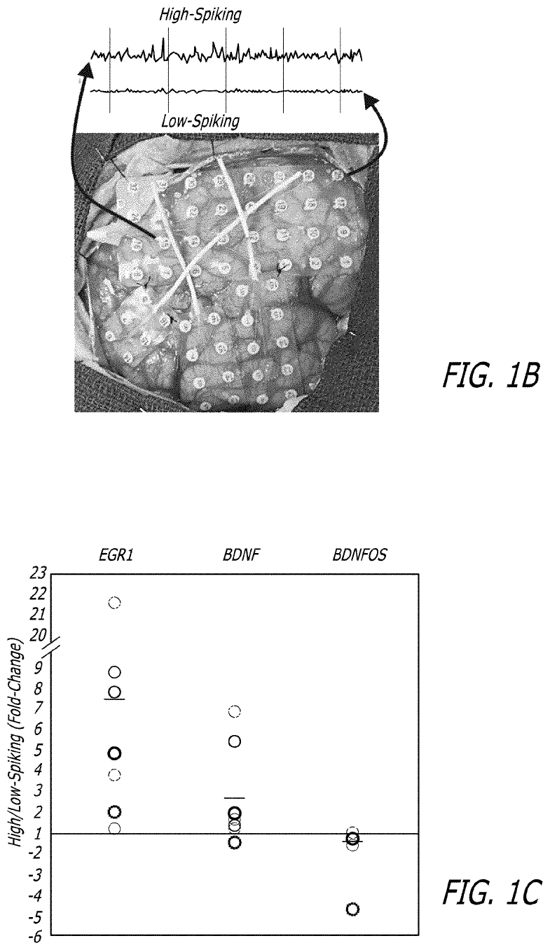

Informed consent was obtained from seven patients who underwent surgery for medically intractable epilepsy (FIG. 1a). Extreme care was taken to ensure that the described study did not influence surgical decision-making. All patients underwent presurgical evaluation and identification of epileptic and control regions as previously described (Beaumont in revision 2011; Rakhade et al. 2005 both incorporated by reference herein for their teachings regarding the same). To localize epileptic brain regions that displayed both clinical seizures and interictal epileptiform discharges (spikes), a two-stage surgical approach using subdural electrodes with continuous brain surface recordings (electrocorticography) and video monitoring was undertaken for two to five days. For these studies, paired tissue samples from neocortex within each patient displaying high and low amount of interictal (between seizures) spiking as determined by quantitative intracranial electrode recordings were used to compare differential gene expression as a function of brain activity (Loeb 2010, incorporated by reference herein for its teachings regarding the same; see also Barkmeier et al., 2012 incorporated by reference herein for its teachings regarding inter-reviewer variability of spike diction on intracranial EEG addressed by an automated multi-channel algorithm). To avoid introducing additional variables into the analysis, each block of tissue was examined histopathologically, and demonstrated a normal 6-layered neocortical structure without lesions. The paired analysis within each patient is critical to isolate the variable under study, which is the degree of activity. Total RNA was prepared using a modification of the protocol described previously (Beaumont in revision 2011, incorporated by reference herein for its teachings regarding the same). The difference was that only gray matter was used by pooling 2-3 nearby strips of gray matter that extended from the pial surface to the white matter from each block of tissue corresponding to a given electrode location. This pooling method helps correct for differences in dissections that could lead to over- or under-representation of specific cortical layers.

Cell Cultures, Transfections, and Depolarizations:

The SH-SY5Y cell line (ATCC) was maintained in Dulbecco's modified Eagle's medium (DMEM) supplemented with 10% FBS and used for experiments. Cells between 17 and 25 passages were transfected with BDNFOS-targeting and BC013641-targeting siRNAs by electroporation according to manufacturer's instructions at approximately 80% confluence (Neon electroporation system, Invitrogen). The electroporation conditions used for SH-SY5Y cell transfection were 1200V, 20 Pulse Width, 2 Pulse numbers, which were optimized using a condition matrix, a control siRNA, and fluorescent reporters (data not shown). Single and multiple depolarizations of cells were induced by adding 100 mM KCl (final concentration) to the medium at different time points as indicated in the figure legend.

qPCR, siRNAs, and Primers:

Total RNA from cultured SH-SY5Y cells was isolated with RNA easy Mini kit according to manufacturer's instructions (QlAgen). The first-strand cDNA was prepared using SuperScript First-Strand cDNA kit (Invitrogen), mRNA and lncRNA expression levels were determined by Taqman quantitative real-time PCR (Taqman qRTPCR). BDNFOS siRNAs named 51, S2, S3, and S4 were custom-designed and synthesized by Invitrogen. The BDNFOS Taqman primer/probe combos were custom-designed by uploading FASTA-format sequences of preferred amplicons regions the ABI Taqman custom design website, and purchased from ABI/Life Technologies. This vendor does not release the actual primer and probe sequences of custom-designed amplicons to the users.

Western Blot Analysis:

Cell lysates were prepared with SDS sample buffer (Sigma) and subjected to Western blotting to measure CREB phosphorylation as described (BEAUMONT in revision 2011, incorporated by reference for its teachings regarding the same). Briefly, proteins separated on 4-20% gradient sodium dodecyl sulfate-polyacrylamide gel were electrically transferred onto nitrocellulose membrane. After blocking with 5% (v/v) skim milk in TBS containing 0.05% Tween-20 (TBST) for 1 hour at room temperature (RT), the membrane was incubated with rabbit polyclonal antibody against p-CREB (Cell Signaling) at dilution of 1:1000 for 1 hour at RT and then with specific secondary antibody coupled with HRP (1:5000) for 1 hour at RT. p-CREB was visualized with ECL detection system (Pierce). The membrane was then stripped and re-probed with CREB antibody (Cell Signaling) at (1:1000) to measure total CREB.

Microarrays:

Seven 60-mer probes per gene, unambiguously mapping by BLAT (KENT 2002, incorporated by reference herein for its regarding the same) to a single genomic location, and free of interspersed and simple repeats, were designed using the Agilent Technologies OpenGenomics eArray interface for 5586 of the 6736 lncRNA genes from Jia et al. (Jia et al. 2010, incorporated by reference herein for its teachings regarding the same). The remaining lncRNA genes were excluded because of eArray failure to yield 7 probes per gene, or because the eArray-designed probes failed a subsequent check for genomic uniqueness and absence of repeats. As a positive control, seven probes each for 111 of the 137 previously determined protein-coding epileptic genes (BEAUMONT in revision 2011) and for 6 housekeeping control genes were included. The eArray Fill Array feature was used to randomly select control protein-coding gene probes to fill all features that would have otherwise remained vacant (<2% of total features on a 44,000-feature, i.e. “44k,” array cell). The entire probeset was printed in quadruplicate on each slide using the Agilent 4×44k high-density oligonucleotide microarray platform.

A dye-flip quadruplicate two-color microarray experiment was performed on each within-patient pair of high-spiking and low-spiking surgically resected samples on both the Agilent human genome-wide array (G4112A) and our custom lncRNA array as described, but using a different labeling method (BEAUMONT in revision 2011). The Epicentre protocol was used to generate aminoallyl-aRNA for subsequent amplification and labeling with either Cy or Alexa dyes. The custom lncRNA arrays were labeled with Alexa dyes (Alexa-647 and Alexa-555, Invitrogen) within the dye-flip design, as described by the manufacturer (SuperScript Indirect RNA Amplification System, Invitrogen) (HOLLOWAY et al. 2008, incorporated by reference herein for its teachings regarding the same). For every patient, each of the quadruplicates was hybridized on four separate slides. Four slides of 4×44k Agilent arrays (4 arrays, each composed of the same set of approximately 44,000 probes) were used to screen seven patients. All slides were scanned as described previously (BEAUMONT in revision 2011).

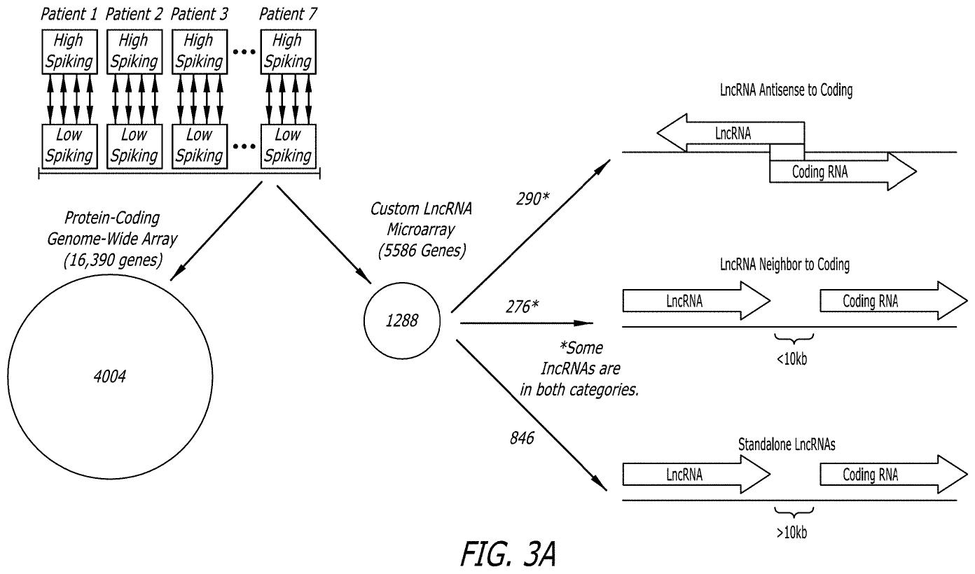

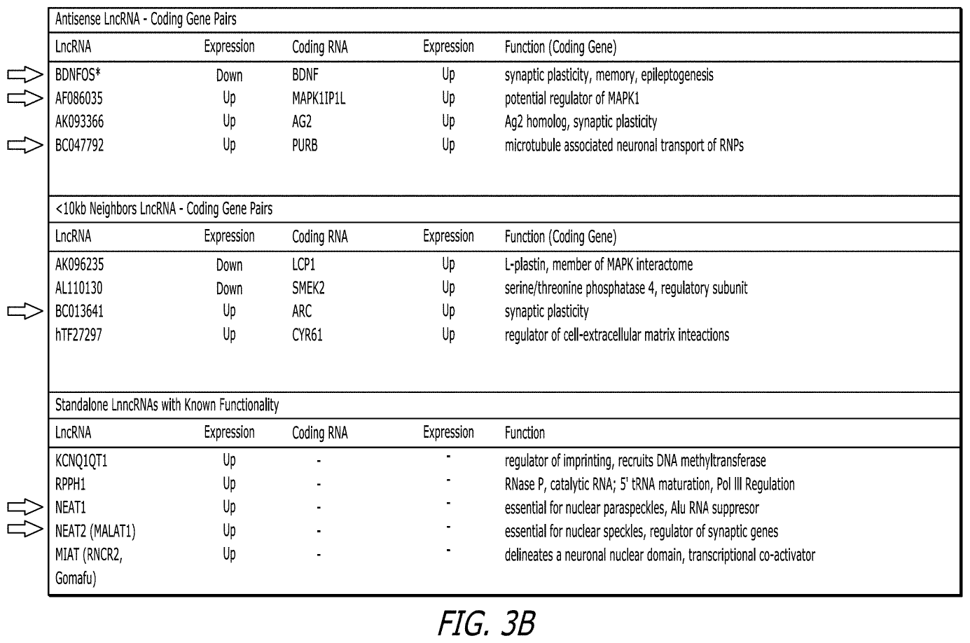

Because the described lncRNA custom microarray platform was new, qPCR was also used to validate a representative subset of differentially-expressed lncRNAs from each of the 3 categories that are indicated by the arrows in FIG. 3b. Which specific probes were responsible for the differential expression of each coding and non-coding gene observed across all seven patients was considered, and probes to target only the region of each transcript that was overlapped by the differentially expressed probes were used. Positive correlation coefficients were seen in all cases, ranging from 0.61 to 0.96 between the array and qPCR results within each patient; all protein-coding gene differential expression results were from the G4112A or F catalog protein-coding microarray and all lncRNA results were from the described lncRNA custom microarray (FIG. 6).

Microarray Statistical Methods:

In order to identify those differentially expressed lncRNAs that may be directly regulating their overlapping or neighboring mRNAs, the described custom lncRNA expression microarray data was integrated with conventional mRNA expression microarray data for the in vivo high/low-activity cortical sample pairs from all 7 patients analyzed with both array types (FIG. 3a). For each epilepsy patient, there was a within-patient sample pair of a high-spiking and a low-spiking region. This within-patient sample pair was analyzed, using the same dye-flip quadruplicate strategy, for both the catalog coding (G4112A) and the custom lncRNA microarray. Differentially expressed genes were identified from both microarray platforms but using the same strategy. Consistency between arrays was first examined by correlating the fold-change of all protein-coding control genes common to both arrays, which was possible because the 111 ‘epileptic transcriptome’ genes from prior protein-coding array work (BEAUMONT in revision 2011, incorporated by reference for its teachings regarding the same) were used as controls on the lncRNA array. The average value of the seven probes corresponding to each control gene on the lncRNA custom array was used. For 140 catalog (Agilent G4112A) coding-array probes corresponding to these 111 genes, Pearson's correlation coefficient was 0.90, attesting to very high reproducibility between the coding array and the non-coding custom array.

Scanned microarray images from coding and noncoding microarrays were analyzed by the software ‘Agilent Feature Extraction (Agilent, V10.3.1)’ with the default protocol ‘GE2_107_Sep09’. A fluorescent correction factor was determined using both Q-RTPCR and Agilent Spike-IN probes. This correction factor was then applied on the fluorescence intensity (fluorescence at exponent 1.125) and improved the fold change prediction. The fluorescence distribution inside each repetition of the microarray experiments was normalized by ‘R’ V2.11 (R DEVELOPMENT CORE TEAM 2010) using the library ‘limma’ (Smyth and Speed 2003, incorporated by reference herein for its teachings regarding the same) in a two-step process: (i) normalization of the intensity of fluorescence between dyes using a Loess correction (iterations=50, span=0.05), and (ii) scaling of the fluorescence intensity on the same range across all the arrays for each dye independently using quantile normalization. The quality of the normalization process of the microarray fluorescence was validated using MA plot density and distribution analysis. Normality was asserted using Anderson-Darling test from the library ‘Nortest’ (Gross 2006, incorporated by reference herein for its teachings regarding the same). For each array, the background level was globally computed using the median of the fluorescence intensity of the negative control probes and subtracted to the signal of each probe.

Once normalized, the microarrays were further analyzed using standard statistical methods (Kerr and Churchill 2001b; Wolfinger et al. 2001, both incorporated by reference herein for their teachings regarding the same). The differentially expressed genes between high and low spiking areas of the brain were determined using a two-step mixed model analyze of variance (Jin et al. 2001, incorporated by reference herein for its teachings regarding the same) with the library LME4 (Bates 2009, incorporated by reference herein for its teachings regarding the same). This mixed model approach has been used to compute the fitted effect and the random effects simultaneously (Littell 1996, incorporated by reference herein for its teachings regarding the same). To improve the sensibility of the analysis (Jin et al. 2001; Kerr et al. 2000, both incorporated by reference herein for their teachings regarding the same), computation did not use the ratio but instead used dye fluorescence intensity indexed by the type of RNA (Tanaka et al. 2000, incorporated by reference herein for its teachings regarding the same) (RNA from high spiking area or RNA from low spiking area). The FDR and corrected p-Value for each gene was computed with ‘R’ using the library ‘fdrtool’ (Strimmer, 2009, incorporated by reference herein for its teachings regarding the same). Differentially expressed genes were detected using fold change and significance simultaneously (Tanaka et al. 2000, incorporated by reference for its teachings regarding the same) and were determined as significantly differentially expressed if their fold change, for at least one probe per gene, was above or equal to 1.4 and if their FDR was equal or lower than 5% in a groupwise analysis with all 7 patients required. These thresholds were selected based on a power analysis using this dye-flip quadruplicate design (LOEB 2009, incorporated by reference for its teachings regarding the same).

To integrate the coding and non-coding transcriptomes of the human neocortex, differentially expressed mRNAs encoded by genomic loci overlapping, or adjacent to, the loci which also encoded differentially expressed lncRNA genes were identified as outlined in FIG. 3a. Specifically, cis-encoded gene pairs in which both a protein-coding gene and a non-coding (lncRNA) gene were expressed from the same locus were identified. These pairs are referred to as lncRNA/mRNA gene pairs. The pairs were then separated into two categories ‘antisense’ and ‘neighbor,’ both of which carry the potential for mRNA regulation by a paired lncRNA (JIA et al. 2010; LIPOVICH et al. 2010, both incorporated by reference for their teachings regarding the same). A cis-antisense gene pair was defined as two genes transcribed from the opposite strands of the same locus in a configuration such that at least some sequence in at least one exon overlaps one exon of the other gene. A neighbor-gene pair was defined as any gene pair such that the nearest boundaries of two nearby, but non-overlapping, genes are less than 10 kb away from one another.

In addition to a number of custom approaches to identify cis-acting coding lncRNA pairs, trans-acting lncRNAs were identified as significant and activity-dependent by their tight correlation (Pearson's correlation coefficient minimum of 0.9) to a well-known group of activity-dependent mRNAs (BEAUMONT in revision 2011; RAKHADE et al. 2007, both incorporated by reference for their teachings regarding the same), which themselves had been co-expressed with a Pearson's correlation coefficient of 0.95. These results were displayed graphically using Cytoscape (SMooT et al. 2011, incorporated by reference for its teachings regarding the same). To include a trans-acting lncRNA in this group, at least one probe (of the seven available probes) representing the lncRNA gene had to meet this statistical requirement.

Co-differential expression was defined as a differential expression profile of two genes such that the differential expression of one gene was either inversely or directly, correlated with the differential expression of the other gene across multiple sample pairs, each of which originated from a different patient and all of which were statistically significant. As can be seen from the gene pairs identified to date, one gene can be in more than one pair.

Results

Reciprocal patterns of BDNF and BDNFOS expression in electrically active human brain. FIG. 1a shows a table of the 7 patients used for the described studies together with quantified in vivo spike frequencies, tissue locations, and pathological descriptions. Patients varied both in sex and age, but were chosen because of the availability of both high and low interictal spiking neocortical brain samples from nearby brain regions for each patient that were removed as part of their seizure surgery treatment. FIG. 1b shows how each of these pairs was selected with a short sample of the in vivo EEG recording that illustrates relative difference in interictal spiking. It is important to emphasize that because of genetic differences, medication effects, and effects of tissue processing the described internally controlled experimental design is crucial. Although patients are listed with different pathological diagnoses from multiple neocortical regions, only tissue samples that showed a normal cortical architecture were used so as not to influence the major variable of interest: increased brain activity.

Because of the potential regulatory relationship of transcripts that code for BDNF with those that encode the partially antisense BDNFOS the relative expression levels of BDNF and BDNFOS between paired high/low spiking regions of human neocortex using quantitative real-time PCR (qPCR) for each patient (FIG. 1c) was compared as a first step. In most patients, BDNF expression was higher in more electrically active regions, whereas BDNFOS lncRNA levels were significantly reduced in the high spiking regions. EGR1 expression was used as a positive control for high spiking human cortical brain regions as its expression has been shown to be directly proportionate to interictal brain activity. These results suggest that increased BNDF levels could in part be regulated by a decrease in antisense-binding BDNFOS.

BDNFOS is a negative regulator of BDNF in an in vitro human cell culture system.

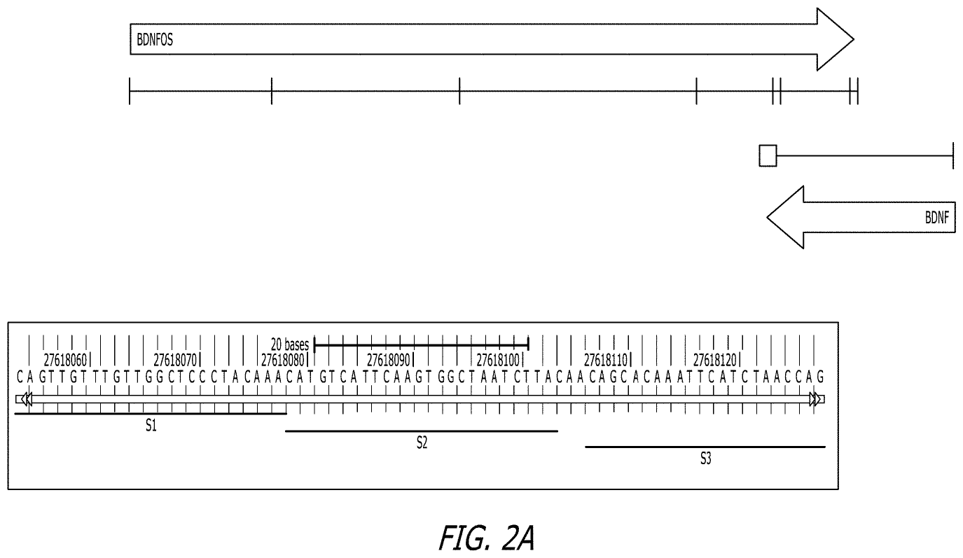

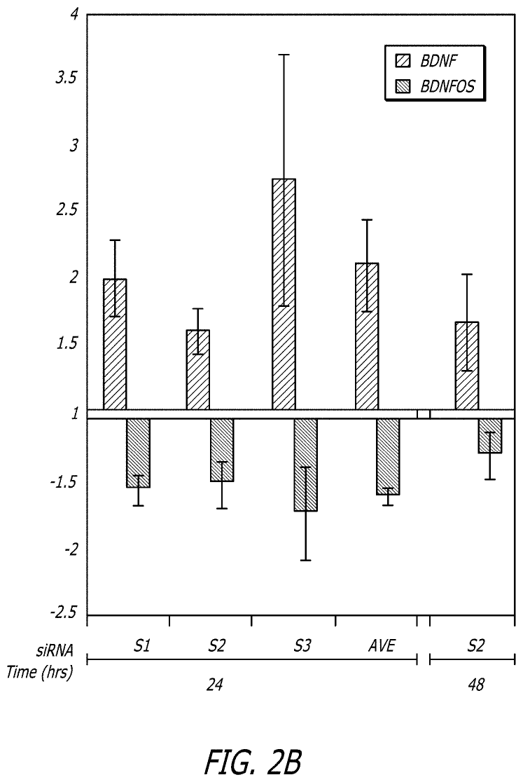

The genomic antisense orientation of BDNF and BDNFOS is shown in FIG. 2a, where both overlapping and non-overlapping regions are delineated. Perturbation of lncRNA levels at multiple cis-antisense lncRNA/mRNA pairs affects levels of the cognate mRNAs. To distinguish whether the lncRNA BDNFOS directly regulates BDNF mRNA levels three siRNAs targeting human BDNFOS (FIG. 2a) were designed and used in qPCR to interrogate BDNFOS lncRNA and BDNF mRNA levels after the siRNA transfections. BDNFOS siRNAs were individually transfected into the human neuroblastoma cell line SH-SY5Y by electroporation, and caused reproducible BDNFOS knockdown at 24 h (all 3 siRNAs) and 48 h (only S2). Two of the siRNAs led to knockdown of BDNFOS by over 70% (FIG. 2b). BDNFOS knockdown by these dsRNAs consistently led to increased BDNF mRNA levels (between 1.5- and 3.5-fold change), suggesting that the cis-antisense BDNFOS RNA functions as a negative regulator of human BDNF (FIG. 2b).

Transcriptome-Wide Profiling of Human Protein-Coding and Long Non-Coding RNAs Reveals Activity-Dependent Gene Pairs.

The functional validation of the primate-specific BDNF/BDNOS pair suggests the potential for many more lncRNA/mRNA and/or lncRNA/lncRNA regulatory relationships across the human genome that may vary as a function of brain activity. The same paired RNA samples from the same 7 patients were thus used to identify members of the activity-dependent coding/noncoding and noncoding/noncoding interactome.

4,004 mRNAs from the catalog array (1,944 upregulated and 2,060 downregulated in high-activity areas) were identified using the provided criterion for differential expression. On the lncRNA arrays, 86 of the 111 positive control genes were upregulated, and 1,288 lncRNA genes were differentially expressed between high-activity and low-activity neocortical regions (698 upregulated lncRNA genes and 590 downregulated lncRNA genes in high-activity areas). BDNF was represented on both the coding microarray and, as a brain-expressed known control gene, on the lncRNA microarray. BDNF was upregulated in high-activity tissue from all 7 patients according to both array platforms: coding microarray, median 3.6-fold change; lncRNA microarray, median 2.8-fold change.

Of the lncRNAs identified to be differentially expressed at high-activity regions, 290 were found at genomic loci which the described genome wide analysis of UCSC cDNA-to-genome and EST-to genome alignments revealed to contain sense-antisense overlaps. At least 4 of these mRNA/lncRNA cis-antisense pairs were co-differentially expressed in all 7 patients (FIG. 3b). Only one of the 4 pairs (BDNFOS/BDNF) identified to date featured an inverse differential expression profile. The other 3 pairs all had a positive, direct-correlation. These pairs include lncRNAs cis-antisense to MAPK1IP1L (MAP Kinase 1 Interacting Protein 1-like, potentially a modulator of MAP Kinase 1, whose role centers on the CREB activation pathway upstream of brain activity-dependent gene expression); PURB (purine-rich element binding protein, a gene expression regulator); and C11ORF96, a human homolog of the rat AG2 gene, induced as a consequence of sustained long-term potentiation in vivo in rat hippocampus and therefore implicated in neuronal plasticity. In summary, the mRNAs of at least 3 of the 4 co-differentially-expressed LncRNA/mRNA cis-antisense pairs have neuronal functions centered on synaptic plasticity.

A set of 276 identified lncRNAs differentially expressed at high-activity brain regions reside at genomic loci corresponding to some of the 808 human mRNA/lncRNA neighbor-gene pairs in which a protein-coding gene and an lncRNA gene were non-overlapping but encoded within 10 kb of each other along the genome (JIA et al. 2010, incorporated by reference for its teachings regarding the same). However, initially only 4 mRNA/lncRNA neighbor gene pairs were identified as co-differentially expressed in the groupwise analysis of the 7 patients (FIG. 3b). These 4 co-differentially-expressed neighbor-gene pairs contained lncRNA genes neighboring the mRNAs ARC (activity-regulated cytoskeleton-associated), a key regulator of neuronal receptor endocytosis required for both synaptic plasticity and long-term memory, L-plastin, relevant to the activity-dependent MAPK/CREB activation by its placement within the human MAPK interactome, SMEK2, a regulatory subunit of Ser/Thr phosphatase 4, and CYR61, a secreted protein that associates with the extracellular matrix and the cell surface, regulates Akt activation, and is differentially expressed in autism.

1,288 lncRNA genes were determined by our custom microarray to be differentially expressed at high-activity areas of the human neocortex. Some of these, including the lncRNA MALAT-1 which is a regulator of several synaptic genes, are indispensable components of specific nuclear bodies, while other lncRNAs regulate imprinting genes and still others perform essential catalytic roles. Differential expression of at least five of these known nuclear RNAs (FIG. 3b, bottom) was significant. MIAT, the sole member of this group which was downregulated in the more active areas, delineates a neuronal nuclear domain and was shown to be both a direct target and a putative co-activator of the transcription factor Oct4. In contrast, the levels of the other four known nuclear lncRNAs were increased in the more active areas. These lncRNAs included: KCNQ1OT1, which may regulate imprinting by recruiting the DNA methyltransferase DNMT1 to differentially methylated regions; RPPH1, the catalytic-RNA component of RNase P, essential for tRNA 5′end maturation and for regulating Pol III-dependent tRNA transcription; NEAT1, an essential component of nuclear paraspeckles which suppresses the nucleocytoplasmic export of Alu-containing RNAs; and NEAT2 (MALAT-1), an essential component of nuclear speckles and a regulator of synaptic genes.

A second unbiased approach was also used to identify activity-dependent lncRNAs with potential importance in synaptic plasticity transcriptional regulatory networks. A number of coding genes including EGR1, EGR2, FOS, and DUSP6 are expressed in human brain in direct relation to the degree of epileptic discharges. Using co-expression clustering of mRNAs, these and other genes that have the same expression pattern across the seven patients were identified. Further, lncRNAs whose pattern of expression correlated with this group of coding genes were identified. FIG. 4, constructed from our coding/non-coding transcriptome quantitation integration by Cytoscape software (SMooT et al. 2011, incorporated by reference for its teachings regarding the same) illustrates co-expression of 26 differentially expressed lncRNA genes with 13 differentially expressed mRNAs. In this diagram, genes that are closer together are more tightly linked. While it appears that there are two linked clusters of coding versus non-coding genes, this apparent separation may in fact be due to the overall lower level of expression of the lncRNAs compared to the mRNAs (by a factor of almost 80-fold). Some of these lncRNAs, such as NEAT1, are not at or near known coding-gene loci (FIG. 3), while others are adjacent to known genes that are not differentially expressed. IL8RBP, a complex mosaic lncRNA transcript that combines unique upstream exons with an IL8RB pseudogene downstream exon, is differentially expressed, although its parental gene IL8RB, and the RUFY4 known gene which the pseudogene overlaps, are not detectable above background in the same samples on our protein-coding gene arrays.

Time-Dependent Patterns of lncRNA/mRNA Co-Differential Expression with Chronic Depolarization of Cultured Human Neuronal Cells.

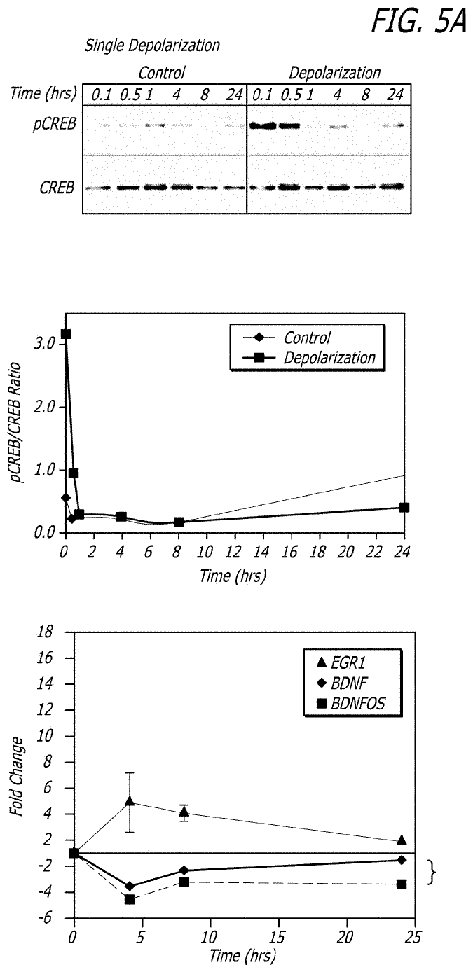

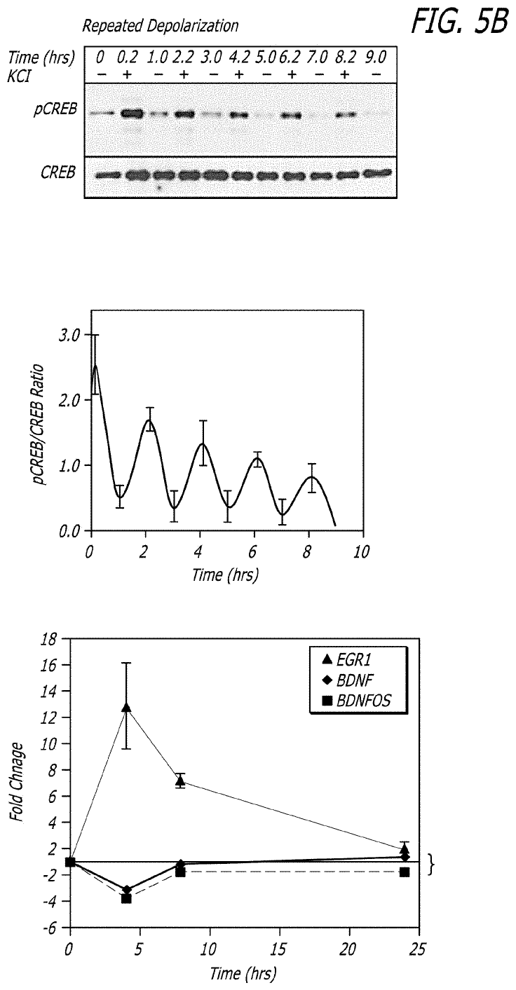

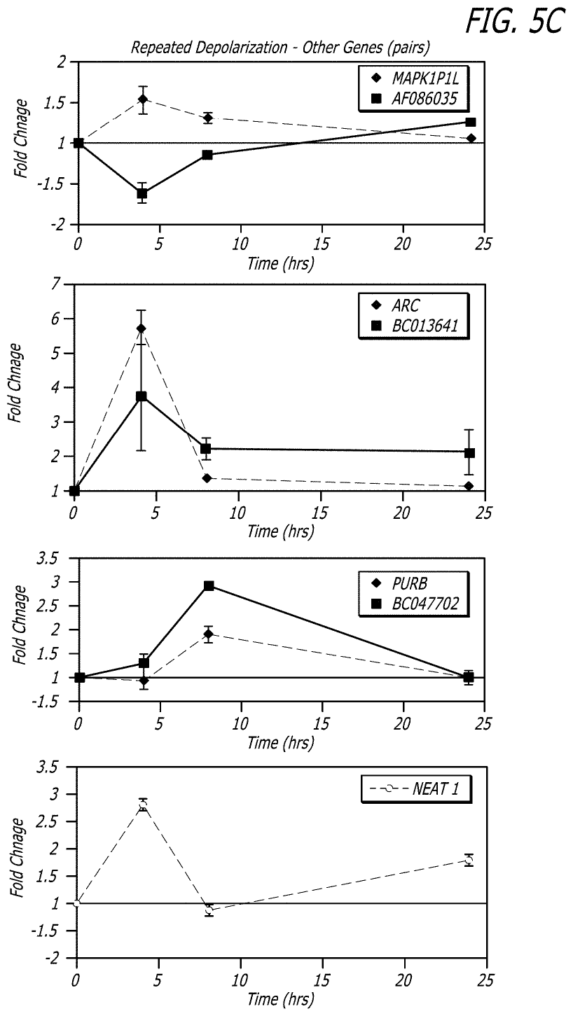

To facilitate the study of primate-specific activity-dependent, lncRNA/mRNA and/or lncRNA/lncRNA gene pairs, an in vitro system of repeated depolarization using the human SH-sy5y neuroblastoma cell line was developed. FIG. 5a shows that while a single treatment of these cells with 100 mM KCl leads to transient CREB activation (phosphor-CREB), repeated 5 min exposures with KCl separated by 2-h intervals lead to more sustained CREB activation, similar to that observed in highly spiking human neocortex and in an animal model of frequent interictal spiking (FIG. 5b).

Gene expression over a 24 h time course together with BDNF and BDNFOS expression by qPCR was compared. Repeated depolarization led to a marked and more sustained increase in EGR1 activation (a marker of epileptic activity in the brain) (FIG. 5b, bottom panels). Even though EGR1 goes up within 4 h, both BDNF and BDNFOS were initially downregulated. However, whereas BDNF and BDNFOS almost return to baseline levels 24 h after a single depolarization, cultures that were repeatedly depolarized showed a small but significant increase in BDNF expression, while BDNFOS remained downregulated. The chronic depolarization-induced reciprocal BDNFOS depletion and BDNF mRNA increase in culture parallels the inverse relationship between BDNFOS and BDNF in high-activity areas of the human brain shown in FIG. 1. Therefore, this cell culture system was applied to interrogate the activity-dependent expression patterns of other lncRNAs and their cis-encoded partner mRNAs from FIG. 3.

In FIG. 5C, chronic depolarization of SH-sy5y cells revealed a number of distinct time-dependent patterns. Unlike the cis-antisense BDNF/BDNFOS pair, the AF086035/MAPK1P1L cis-antisense pair had an opposite effect of decreasing lncRNA and increasing mRNA levels early in the time course, although by 24 h the lncRNA level showed a slight increase. The BC013641/ARC neighbor-gene pair displayed increased expression of both genes at the 4 h timepoint in the depolarization-treated Sh-sy5y cells, mirroring the increased expression of both genes in the high-activity human brain. At subsequent time points, ARC mRNA levels decreased back to the pre-treatment levels although a sustained elevated level of the BC013641 lncRNA encoded near the ARC gene along the genome (FIG. 5C) was observed. Since the BC013641 gene is located approximately 6 kb from ARC with a divergent genomic orientation relative to ARC, two custom siRNA oligonucleotides targeting BC013641 were designed. Although both siRNAs knocked down BC013641, only one led to a 1.25-fold increase in the mRNA level of the neighboring ARC gene, suggesting that reciprocal gene expression directionality at lncRNA/mRNA pairs may occur at neighbor-gene loci such as BC013641/ARC, and not solely at sense-antisense loci such as BDNFOS/BDNF (data not shown). The BC047792/PURB cis-antisense pair showed increased expression of both genes, which mirrored the coordinate increase observed in high-spiking brain regions, however, in contrast to BC013641/ARC, expression of both transcripts were maximal at the 8 h time point and returned to baseline at 24 hours, showing no sustained increase in lncRNA expression. Finally, the time course of one lncRNA, NEAT1, that has a potentially far-reaching regulatory role was examined. NEAT1 goes up within 4 h, returns to baseline at 8 h, but shows some chronic elevated expression at 24 h. NEAT1 is not in a cis-encoded pair, but it is in a trans-encoded network as one of the two targets of RPPH1, another lncRNA. Therefore, RPPH1/NEAT1 are a trans-encoded lncRNA/lncRNA pair. RPPH1/NEAT2 (synonym of NEAT2 is MALAT-1) are another trans-encoded lncRNA/lncRNA pair.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

### REFERENCES

BARKMEIER et al., 2012 High inter-reviewer variability of spike detection on intracranial EEG addressed by an automated multi-channel algorithm, Clin Neurophysiol, 123(6):1088-95.
BATES et al., 2009 Ime4: Linear mexed-effects models using S4 classes, pp. R Foundation for Statistical Computing, available online at r-forge.r-project.org/R/?group_id=60.
BEAUMONT et al., in revision 2011 Layer-specific CREB target gene induction in human neocortical epilepsy.
GROSS, 2006 nortest: Tests for Normality. R package version 1.0., pp. R Foundation for Statistical Computing, available online at cran.r-project.org/web/packages/nortest/.
HOLLOWAY et al., 2008 Liver-specific hepatocyte nuclear factor-4alpha deficiency: greater impact on gene expression in male than in female mouse liver. Mol Endocrinol 22: 1274-1286.
JIA et al., 2010 Genome-wide computational identification and manual annotation of human long noncoding RNA genes. RNA 16: 1478-1487.
JIN et al., 2001 The contributions of sex, genotype and age to transcriptional variance in Drosophila melanogaster. Nat Genet 29: 389-395.
KERR et al., 2001a Experimental design for gene expression microarrays. Biostatistics 2: 183-201
KERR et al., 2001b Statistical design and the analysis of gene expression microarray data. Genet Res 77: 123-128.
KERR et al., 2000 Analysis of variance for gene expression microarray data. J Comput Biol 7: 819-837.
LITTELL et al., 1996 SAS System for Mixed Models. SAS Institute, North Carolina, USA.
LOEB, J. A., 2010 A human systems biology approach to discover new drug targets in epilepsy. Epilepsia 51 Suppl 3: 171-177.
LOEB & BEAUMONT, 2009 What goes in is what comes out: The design and analysis of microarray experiments, pp. 213 in Bioinformatics for Systems Biology, edited by S. KRAWETZ. Humana Press, New York, N.Y.
R DEVELOPMENT CORE TEAM, 2010 R: A language and environment for statistical computing, pp. R Foundation for Statistical Computing, available on the World Wide Web at r-project.org/, Vienna, Austria.
RAKHADE et al., 2005 A common pattern of persistent gene activation in human neocortical epileptic foci. Ann Neurol 58: 736-747.
SMOOT et al., 2011 Cytoscape 2.8: new features for data integration and network visualization. Bioinformatics 27: 431-432.
SMYTH & T. SPEED, 2003 Normalization of cDNA microarray data. Methods 31: 265-273.
SONE et al., 2007 The mRNA-like noncoding RNA Gomafu constitutes a novel nuclear domain in a subset of neurons. J Cell Sci 120: 2498-2506.
STRIMMER, K., 2009 fdrtool: Estimation and Control of (Local) False Discovery Rates. R package version 1.2.6, pp. R Foundation for Statistical Computing, available online at strimmerlab.org/software/fdrtool/.
TANAKA et al., 2000 Genome-wide expression profiling of mid-gestation placenta and embryo using a 15,000 mouse developmental cDNA microarray. Proc Natl Acad Sci USA 97: 9127-9132.
WOLFINGER et al., 2001 Assessing gene significance from cDNA microarray expression data via mixed models. J Comput Biol 8: 625-637.
YAO et al., 2004 Accuracy of cDNA microarray methods to detect small gene expression changes induced by neuregulin on breast epithelial cells. BMC Bioinformatics 5: 99.

We claim:

1. A method of identifying a therapeutic target in human brain tissue, the method comprising:
obtaining at least one pair of two samples from a live human brain, wherein each sample in each pair has a different level of electrical brain activity from the other sample in the pair;
measuring expression of long non-protein-coding RNA (lncRNA) molecules (lncRNAs) and protein-coding messenger RNA (mRNA) molecules (mRNAs) in the two samples;
identifying one or more lncRNAs that have differential expression between the two paired samples;
identifying one or more mRNAs that have differential expression between the two paired samples; and
identifying an activity dependent lncRNA/lncRNA or lncRNA/mRNA gene pair utilizing a method comprising:
identifying a first differentially expressed lncRNA with a genomic location that overlaps with, or is within 10 kb of the genomic location of a differentially expressed mRNA or a second differentially expressed lncRNA,
wherein the activity dependent lncRNA/lncRNA or lncRNA/mRNA gene pair comprises the therapeutic target.

2. The method of claim 1, wherein the electrical brain activity is classified as high or low based on the frequency and/or amplitude of interictal and ictal spiking.

3. The method of claim 1, wherein the detecting expression of lncRNAs and mRNAs in the samples utilizes at least one microarray capable of quantifying lncRNA expression and mRNA expression.

4. The method of claim 1, wherein the detecting expression of lncRNAs and mRNAs in the paired samples utilizes at least one microarray capable of quantifying lncRNA expression and at least one microarray capable of quantifying mRNA expression, wherein consistency of differential expression data between the at least one lncRNA microarray and the at least one mRNA microarray is evaluated by correlating the fold-change of protein-coding control genes common to both arrays.

5. The method of claim 1, wherein the detecting expression of lncRNAs and mRNAs in the paired samples comprises:
contacting the two samples with a set of lncRNA probes and a set of mRNA probes and detecting binding between each of the probes and its target, wherein the set of lncRNA probes comprise probes that target at least 1000 lncRNAs, and the set of mRNA probes comprise probes that target at least 4000 mRNAs;
identifying lncRNAs that have differential binding to their probes, as compared between the two samples; and
identifying mRNAs that have differential binding to their probes, as compared between the two samples.

6. The method of claim 5, wherein the detecting expression further comprises contacting the pair of samples with dyes before contacting the samples with the probes, wherein each member of the paired samples is contacted with a different dye than the other member.

7. The method of claim 1, wherein the detecting expression utilizes a dye flip microarray.

8. The method of claim 1, wherein the detecting expression further comprises generating aminoallyl-aRNA from RNA in each sample before measuring expression.

9. The method of claim 5, wherein the at least 1000 lncRNAs are each targeted by at least 7 non-identical probes, and wherein the at least 4000 mRNAs are each targeted by at least 7 non-identical probes.

10. The method of claim 1, wherein the therapeutic targets are epilepsy targets, and the human brain is from a human epilepsy patient.

11. A method for identifying putative therapeutic targets, the method comprising:
obtaining at least one pair of brain tissue samples from a live human, wherein each member of the pair has a different level of electrical brain activity from the other member;
measuring expression of long non-protein-coding RNA (lncRNA) molecules (lncRNAs) and protein-coding messenger RNA (mRNA) molecules (mRNAs);
identifying lncRNAs and mRNAs that are differentially expressed between the members of each individual sample pair; and
linking a first differentially expressed lncRNA with a differentially expressed mRNA and/or a second differentially expressed lncRNA when the gene encoding the first differentially expressed lncRNA overlaps with, or is adjacent to, the gene encoding the differentially expressed mRNA and/or the gene encoding the differentially expressed second lncRNA along the human genome, thereby identifying an lncRNA/mRNA gene pair and/or an lncRNA/lncRNA gene pair as putative cis-encoded therapeutic targets; and/or
linking a first differentially expressed lncRNA with a differentially expressed mRNA and/or with a second differentially expressed lncRNA when the differentially expressed first lncRNA and the differentially expressed mRNA and/or second lncRNA are encoded at different genomic loci, thereby identifying an lncRNA/mRNA gene pair and/or an lncRNA/lncRNA gene pair as putative trans-encoded therapeutic targets.

12. The method of claim 11, wherein the linking of differentially expressed lncRNA with differentially expressed mRNA and/or lncRNA further requires that the differential expression of the lncRNA and mRNA or lncRNA and lncRNA be observed in more than one brain sample pair, each pair having a low electrical brain activity member and a high electrical brain activity member.

13. The method of claim 1, wherein electrical brain activity is classified as high or low based on the frequency and/or amplitude of interictal and ictal spiking.

14. The method of claim 1, wherein differential expression is identified by quantifying lncRNA and mRNA expression.

15. The method of claim 14, wherein the expression quantification utilizes at least one microarray capable of quantifying lncRNA expression and mRNA expression.

16. The method of claim 14, wherein the quantifying utilizes at least one microarray capable of quantifying lncRNA expression and at least one microarray capable of quantifying mRNA expression wherein consistency of differential expression data between the at least one lncRNA microarray and the at least one mRNA microarray is evaluated by correlating the fold-change of protein-coding control genes common to both arrays.

17. The method of claim 14, further comprising evaluating the putative therapeutic target as a molecular site of effective intervention.

18. The method of claim 17 wherein the therapeutic target of a pair is lncRNA, mRNA and/or both.

19. The method of claim 11, wherein the putative therapeutic targets are epilepsy targets, and the live human is an epilepsy patient.
